(19) United States
(12) Patent Application Publication
(10) Pub. No.: US 2008/0175891 A1
(43) Pub. Date: Jul. 24, 2008

(54) METHODS, COMPOSITIONS, AND FORMULATIONS FOR PREVENTING OR REDUCING ADVERSE EFFECTS IN A PATIENT

(76) Inventor: Richard R. Stover, New York, NY (US)

Correspondence Address: WILSON SONSINI GOODRICH & ROSATI, 650 PAGE MILL ROAD, PALO ALTO, CA, 94304-1050, US

(21) Appl. No.: 11/971,396

(22) Filed: Jan. 9, 2008

Related U.S. Application Data
(60) Provisional application No. 60/879,664, filed on Jan. 9, 2007.

(51) Int. Cl. A61K 9/70; A61P 9/10

(52) U.S. Cl. 424/449

(57) ABSTRACT

The present invention provides transdermal administration of AICA riboside, or a prodrugs, analogs, or salts thereof, and/or a blood clotting inhibitor for preventing or reducing adverse side effects in a patient. The type of patient that may benefit includes a patient with decreased left ventricular function, a patient with a prior myocardial infarction, a patient undergoing non-vascular surgery, or a fetus during labor and delivery.

### CROSS-REFERENCE

This application claims the benefit of U.S. Provisional Application No. 60/879,664, filed Jan. 9, 2007, which is incorporated herein by reference in its entirety.

### BACKGROUND OF THE INVENTION

Several types of ischemia exist including myocardial, mesenteric, and cerebral. It is known that the purine nucleoside, adenosine, can exert a protective effect under ischemic conditions. Acadesine or 5-aminoimidazole-4-carboxamide (AICA) riboside, a precursor molecule of nucleotide biosynthesis, can enhance the local endogenous levels of extracellular adenosine during periods of ischemia. AICA riboside enters cells and is phosphorylated to AICA riboside monophosphate (“ZMP”), a naturally occurring intermediate in purine biosynthesis. AICA riboside increases extracellular adenosine levels under conditions of net ATP breakdown and, therefore, in light of the cardioprotective and neuroprotective properties of adenosine it may have potential therapeutic uses.

It would be useful to find convenient and therapeutically effective methods of administering formulations of AICA riboside and/or analogs thereof which can be used to treat/prevent ischemic conditions, conditions regulated by adenosine, effects of reduced blood flow to a tissue or simply prevent morbidity/mortality in a patient.

The present invention is directed to transdermal administration of AICA riboside and AICA riboside analogs.

### SUMMARY OF THE INVENTION

The present invention relates to methods of preventing or reducing adverse effects in a patient, including by transdermally administering acadesine or a prodrug, analog, or salt thereof; or acadesine or a prodrug, analog, or salt thereof and a blood clotting inhibitor. The invention may benefit several types of patients, including a patient with decreased left ventricular function, a patient with a prior myocardial infarction, a patient undergoing non-vascular surgery, or a fetus during labor and delivery.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The term “co-administered” as used herein refers to administration of two or more agents as part of the same treatment plan, whether or not simultaneous in time or not. Thus, agents that are co-administered can be co-formulated or independently formulated. Preferably, two agents are co-administered such that their biological activity overlaps in time.

The term “patient” is preferably a mammal, and more preferably a human.

The term “treat”, “treating” or “treatment” as used herein refers to prevention as well as amelioration or reduction of a symptom or a condition affecting an organism.

In one aspect, the invention provides a method of preventing or reducing adverse effects in a patient with decreased left ventricular function having an ejection fraction of less than 30% by administering an effective amount of acadesine, or a prodrug, analog or salt thereof. Another embodiment provides a method where the patient is female and/or or between the age of 65 and 95.

Another aspect of the invention provides a method of decreasing tissue damage associated with decreased blood flow in a patient by administering an effective amount of acadesine, or a prodrug, analog, or salt thereof, wherein the patient is a fetus during labor and delivery. In one embodiment, an effective amount of acadesine, or a prodrug, analog, or salt thereof is administered to the woman delivering the fetus.

In one aspect, the invention provides a pharmaceutical embodiment, the invention provides a pharmaceutical formulation comprising acadesine, or a prodrug, analog, or salt thereof for use in administration to a fetus during labor and delivery to prevent or reduce tissue damage associated with decreased blood flow in the fetus.

Novel methods are described for enhancing adenosine release, especially during net ATP catabolism, i.e., during a time of a decreasing or decreased ratio of ATP synthesis to ATP breakdown in cells or cellular compartments.

Methods for enhancing adenosine release utilize the administration of compounds which are believed to alter one or more of the biochemical pathways of adenosine metabolism so that the net result is an enhanced extracellular concentration of adenosine (resulting from one or more processes, including enhanced intracellular production and/or release of adenosine). Examples of compounds useful in the invention include compounds broadly classified as purine nucleosides and related analogs, such as AICA riboside, AICA ribotide, 1-β-D-ribofuranosyl-1H-1,2,4-triazole-3-carboxamide (ribavirin), ribavirin monophosphate, and various pro-forms of the above compounds. The compounds are taken up by cells and, if necessary, are believed to be converted to their monophosphate and, to a lesser extent, their triphosphate forms. Also included are (1) agents that can enhance endogenous synthesis of AICA ribotide or metabolites, such as purine intermediary metabolites or compounds that can form these metabolites, e.g., succinylaminoimidazole carboxamide (SAICA) riboside, (2) agents that cause a buildup of AICA-ribotide or its metabolites, including methotrexate, and (3) agents that cause bacterial flora to increase AICA riboside production, such as sulfonamides. These compounds can be administered to a patient either prophylactically, in some cases, and/or in direct response to a bodily condition in others. Purine nucleosides that enhance the excretion of cellular adenosine and/or adenosine analogs may be administered to a living system over the concentration range of 0.5 micromolar to 0.5 molar and, typically, are administered in concentrations up to 0.5 molar.

Adenosine or inosine are generated from adenosine triphosphate n the course of rapid cellular energy utilization, such as during seizure activity, arrhythmias, or a condition resulting in decreased blood flow (ischemia), such as a stroke, heart attack, or angina. Normally, during such an event, the production of inosine is greater than that of adenosine. In the area of low flow during coronary occlusion, for example, the ratio of venous inosine to adenosine is approximately 100 to 1. A certain percentage of inosine and adenosine exit the cell and are present in the immediate extracellular environment. The compounds useful in the methods described and claimed herein have been shown to enhance the extracellular concentration of adenosine, and the production of inosine has been shown to be decreased. Adenosine levels are not altered significantly throughout the patient because alterations in adenosine production only occur in areas of, and at the time of, net ATP use and because adenosine is rapidly degraded. Thus, the methods described and claimed herein will cause a localized increased concentration of extracellular adenosine instead of a systemic or generalized adenosine enhancement.

Oxidation of low density lipoprotein (LDL) is one of the first, if not the first, steps in the process of atherosclerosis, a process believed to involve inflammation and to be due to mononuclear cell and/or granulocyte activation. The oxidized lipids are taken up by macrophages to form the atherosclerotic plaque. Because adenosine prevents the production of superoxide radicals by granulocytes, the compounds of the invention which enhance adenosine release should slow, prevent, or reverse the development of atherosclerosis.

Patients that are suffering from (1) autoimmune disease, (2) arthritis, (3) psoriasis, (4) organ transplant rejection, (5) complement-mediated granulocyte activation after exposure to heart-lung or dialysis membranes, (6) ARDS, or other inflammatory conditions, Whether due to granulocyte activation (as (1)-(6) above can be) or mononuclear cell activation, should also experience relief on treatment With the compounds useful in the invention because ATP catabolism is expected during an inflammatory response.

Patients suffering from diseases which may be associated with chronic low adenosine, such as insomnia, autism, schizophrenia and cerebral palsy, will also benefit from the use of the invention to increase adenosine concentrations.

Further, treatment with compounds of the invention will benefit patients suffering from a variety of illnesses relating to mast cell degranulation. They include individuals suffering from allergies, particularly asthma, hay fever, chronic urticaria, urticaria pigmentosa and eczema. Both AICA riboside and ribavirin, for example, suppress mast cell activation, including the prevention of mast cell degranulation. Decreased mast cell activity will also benefit patients with reduced blood flow because agents released from mast cells can increase damage during ischemia through processes such as arrhythmias or vessel spasm.

It is anticipated that compounds useful in the invention will be effectively administered in amounts ranging from about 0.1 mg/kg/day to about 500 mg/kg/day, preferably from about 15 mg/kg/day to about 200 mg/kg/day. That range of dosages should be especially suitable for compounds useful in the invention as prophylactics for the prevention of tissue damage associated with undesired restricted or decreased blood flow. The use of at least about 0.1 mg/kg/day of AICA riboside or AICA ribotide, preferably from about 1.0 mg/kg/day to about 500 mg/kg/day for said prophylaxis and, more preferably, from about 20 mg/kg/day to about 100 mg/kg/day, is further anticipated. Also contemplated for said prophylaxis is the administration of ribavirin or ribavirin monophosphate in an amount of at least about 0.1 mg/kg/day, preferably from about 1.0 mg/kg/day to about 20 mg/kg/day. In the case of treatment of brain diseases, such as stroke, seizures, epilepsy, transient ischemic attack, autism, schizophrenia, cerebral palsy and insomnia, a dosage of more than 200-500 mg/kg/day may be needed because of the blood/brain barrier. The use of brain-directed pro-drugs may, however, enable a lower dosage.

Because the purine nucleoside AICA riboside can be metabolized to uric acid, this agent may b used with allopurinol or other drugs that prevent uric acid synthesis, or with a uricosuric agent such as probenicid. Certain agents, such as methotrexate and ribavirin, whose metabolites inhibit AICA ribotide transformylase, may cause an elevation of endogenously synthesized AICA ribotide and create effects similar to administering the purine nucleoside. Concomitant administration of AICA riboside or AICA ribotide with an inhibitor of AICA ribotide transformylase should have at least additive effects. In addition, any one of the de novo purine nucleotide synthesis intermediates (after the first committed step for purine synthesis) or their nucleosides or bases can be assumed to be rapidly converted to AICA ribotide. An example is SAICA ribotide or its nucleoside or base.

The compounds can be used to enhance extracellular concentrations of adenosine and, therefore, to treat diseases that arise from, or are aggravated by, insufficient blood flow through a particular organ or portion thereof. For example, heart attacks or strokes, the microvascular disease of diabetes mellitus (which can affect the brain, the kidney, the heart, the skin, the retina, and the peripheral nerves and their associated microvasculatures), or events resulting in a less prolonged loss of blood flow, such as angina pectoris, transient ischemic attacks, bowel ischemia, kidney ischemia, intermittant claudication of skeletal muscle, migraine headaches, and Raynaud's phenomenon can be treated by administering the compounds of the invention. Adenosine is known to be both a potent vasodilator, which acts by reducing vascular smooth muscle contraction, and an inhibitor of granulocyte free radical production, a process involved in ischemic injury. As noted, it should also be useful in the treatment of atherosclerosis.

Upon contact with cells, it is believed that the compounds useful in the invention enter the cell where they can be phosphorylated by adenosine kinase or, in the case of administration of base, they can be converted to a nucleotide by a phosphoribosyl transferase enzyme to yield a purine nucleotide monophosphate, and eventually also the nucleoside triphosphate. The triphosphate form may comprise a pool for breakdown to the monophosphate form.

While not wishing to be bound by the following proposed mode of action, it is postulated that the compounds of the invention, or their metabolites, inhibit one or more enzymes in the adenosine biologic pathway, including AMP deaminase, thus shunting ATP more toward the cellular production, release, and less re-uptake of adenosine, and shunting it away from, concomitantly, the cellular production and release of inosine.

It is important to note that ribavirin cannot be metabolized into normal purines, i.e., it does not become AMP, ADP, ATP, IMP, or the guanosine phosphates GMP, GDP, or GTP. In other words, the compounds useful in the invention can enhance adenosine release without being directly metabolized into adenosine. AICA riboside has biochemical properties similar to ribavirin and appears to enhance adenosine release by a similar mechanism rather than by a circuitous conversion to adenosine. The compounds have been shown not to act by the repletion of ATP pools.

A suggested pathway enabling the beneficial effect seen with compounds useful in the invention is the entry of such compounds into the cell, where they become ribosylated (if the sugar ring is not yet present) and phosphorylated (if not yet phosphorylated) to their monophosphate form. The monophosphate forms of the claimed compounds inhibit AMP deaminase. During ATP catabolism, the AMP pool increases more in treated cells than in untreated cells because AMP is no longer able to move as readily to IMP. Cleavage of the purine monophosphates results in a higher cellular release of adenosine with a concomitant lower cellular release of inosine. Because adenosine appears to be a natural beneficial mediator during certain pathological events, enhancement of its release by channeling ATP to adenosine instead of inosine is a novel and extremely important method of treatment.

During a heart attack, adenosine is normally released and it assists in maintaining the patency of ischemic vessels through vasodilation and inhibition of granulocyte free radical production and concomitant microvascular plugging, as described below. The compounds useful in the invention enhance adenosine release and, therefore, enhance the normal protective effect of adenosine during such an ischemic event.

While the release of adenosine is at times a beneficial event, high levels of adenosine in areas where it is not required can be detrimental. One virture of the invention described and claimed herein is that the patient is not treated with adenosine itself and the compounds useful in the invention selectively increase adenosine release from cells in which there is a net ATP breakdown. Thus, only cells in the vicinity are treated. Treatment of patients with compounds useful in the invention allows the targeting of enhanced adenosine release specifically to tissue undergoing net ATP catabolism, i.e., to tissue which is in need of adenosine release. The systemic effects of adenosine administration are avoided. Further, adenosine is released only at the specific time it is needed. All diseases and pathologic states described or disclosed herein involve or are believed to involve localized net ATP catabolism.

Additionally, cells that would respond beneficially to adenosine are more responsive than they would be if they were continually bathed in higher concentrations of adenosine. Because adenosine is available only instantaneously when it is needed, receptors on the surfaces of cells, such as granulocytes and smooth muscle cells, are not continually exposed and, therefore, have a much larger response, as their adenosine receptors have not been down-regulated by continual adenosine exposure.

In addition to acting to cause vasodilation through the release of adenosine, the compounds of the invention can increase collateral blood flow by a second mechanism. Studies have shown that in the region of restricted blood flow, granulocytes become activated, release oxygen-free radicals, and subsequently stick in and damage microvasculature. Drugs useful in the invention through enhanced adenosine release prevent granulocytes from producing the free radicals and, therefore, granulocytes stick less in the microvessels, which allows blood flow from collateral vessels into the blocked area.

If the drug is introduced into a patient to reach an ischemic region after or during an event causing that ischemia, there is little or no ability to direct ATP to adenosine at that site because the target ATP pools are depleted relatively quickly. Also, because many of the damaging events during ischemia occur rapidly, the drug should ideally be present at the earliest possible moment. With the drug present as a prophylactic agent, there is also the possibility that the process sought to be interrupted can be slowed early enough to prevent any permanent damage. For example, the increased microvascular blood flow from vasodilation and decreased white cell sticking could maintain microvascular patency, as well as in a sense help wash away clots, clot-promoting material, or other deleterious agents from the proximal atherosclerotic regions.

Other factors make it important to administer the drug before or during an ischemic event. If a drug is administered after a blockage, it is less able to reach the tissue involved because there is little or no blood flow to this area. This is an energy-requiring reaction utilizing ATP. If ATP is not available because of high metabolic activity and/or increased ATP destruction, then the AICA riboside or a similar drug cannot be made into its active form. In addition, during rapid ATP breakdown, the inosine in the cell may be significantly competing with the drug for entry into the cell, both compounds being purine nucleoside analogs.

Further, compounds of the invention are envisioned to be beneficial in combination with certain other treatment modalities, as described below. As compounds of the invention, when taken prophylactically, enhance adenosine release during an acute ischemic event, a heart-attack patient undergoing such treatment would have a greater chance of not dying of a sudden arrhythmia before entry to a hospital. In addition, the microvascular bed would be protected during the time the patient is in transit to the hospital and before additional therapy can be instituted.

Often, an acute ischemic event is silent for some time, and there is an additional delay before the patient realizes what is happening and help is sought. When medical help reaches the patient, of course, as when an ambulance arrives or when the patient reaches a hospital, the patient can be given thrombolytic therapy. Thrombolytic therapy, such as the infusion of tissue plasminogen activator (t-PA), streptokinase, urokinase, or anticoagulants such as heparin or Coumadin, are all aimed at opening up a proximal occlusion, such as occurs during a heart attack or stroke. Currently, the patient needs to receive this treatment within about four hours of an acute ischemic event. After several hours, there is irreversible damage to the tissues, especially the microvascular bed. If the patient is prophylactically taking AICA riboside or another compound of the invention, the patient's microvascular bed will be protected longer because of the presence of enhanced adenosine.

The enhanced adenosine release prevents superoxide free radical production and/or granulocyte plugging and damage to the microvessels. Therefore, the patient should be protected for a longer period of time after the acute ischemic event. For example, for perhaps 8-16 hours after a cardiovascular occlusion, it would still be possible to institute one of these thrombolytic therapies in order to open a proximal lesion. Again, opening a proximal lesion is only beneficial if the downstream microvessels are able to be perfused.

Compounds useful in the invention will also be beneficial in combination with thrombolytic agents, such as tissue plasminogen activator, as well as with other agents which are either free radical scavengers or prevent the production of free radicals. Examples of free radical scavengers are superoxide dismutase, a protein which is infused after an ischemic event, or materials which have less proven efficacy, such as catalase, acetylcysteine (mucomyst), vitamin E, gluthathione, and selenium. Examples of compounds which are thought to prevent free radical production are allopurinol by its inhibition of xanthine oxidase, and icosopentanoic acid by its down regulation of prostaglandin metabolites and, finally, antibodies against certain receptors on activated granulocytes which prevent their sticking in microvessels. Compounds useful in the invention, through elevated adenosine, inhibit the NADPH oxidase free radical-generating system of granulocytes and should, therefore, also be useful when combined with agents such as allopurinol, which inhibits free radical production from xanthine oxidase.

Another disease caused by or able to cause restricted blood flow is myocardial arrhythmia. Although restricted blood flow can initiate the onset of arrhythmia, the precise cause is unknown. However, it is known that lipid peroxidation by oxygen radicals is arrhythmogenic. Since the latter are produced by granulocytes, the inhibition of granulocyte superoxide production by the method of the invention can be expected to control arrhythmia. In addition, mast cells are in higher concentration in areas of atherosclerosis. Suppression of their activation might reduce the release of other mediators of arrhythmias. Adenosine also has direct anti-arrhythmic effects on myocytes. The prophylactic effect of AICA riboside treatment on arrhythmias was demonstrated by Examples VI and XIV, the results showing a decreased number of premature ventricular depolarizations and ventricular tachycardia episodes. Rapid firing of cells during arrhythmia causes increased net ATP catabolism and adenosine release.

The adenosine released from neuronal cells when they are stimulated and break down ATP during seizure (epileptic) activity normally will feedback and suppress this seizure (epileptic) activity. In the presence of compounds useful in the invention, the amount of suppression of a seizure event should be significantly increased. Example XIII demonstrates that AICA riboside causes a decreased incidence and prolonged latency to pentylene tetrazol-induced seizures.

Patients that are suffering from autoimmune diseases, arthritis, or other inflammatory conditions should also experience relief if treated with purine nucleosides or analogs useful in the invention because ATP catabolism is expected during the increased cellular excitation associated with an inflammatory response. Inflammatory diseases occur naturally in man and appear to involve an immune reaction to an individual's own tissues. For an autoimmune response to be mounted, it is required that different immune cells interact to support the response. Thus, chemicals that interfere with the requisite cell-cell interactions can be expected to interfere with the course of the disease. One immune cell type necessary for the generation of an autoimmune response is the lymphocyte. Because adenosine is well known to be suppressive to lymphocytes, administering compounds useful in the invention, such as AICA riboside or ribavirin, should inhibit or deplete this population of immune cells during an inflammatory episode, and thus be of considerable therapeutic benefit to inflammatory disease sufferers. Also, as noted, adenosine inhibits granulocyte production of oxygen-free radicals and adherence to endothelial cells, both of which appear to be important factors in many inflammatory processes, such as autoimmune diseases.

Conditions potentially associated with chronic low adenosine may also be treated by compounds of the invention. These pathologic states include autism, insomnia, cerebral palsy, schizophrenia, and other neuropsychiatric symptoms. It is anticipated that doses ranging from 0.1 mg/kg/day up to about 200 mg/kg/day will be beneficial. The results of therapeutic trials with AICA riboside in patients with adenylosuccinase deficiency (autism) are shown in Example X. The oral administration of AICA riboside at a single dose of 5 mg/kg/day, increased to 2×5 mg/kg/day and, finally, to 2×10 mg/kg/day, showed a clear-cut improvement in one of two patients, both patients being described as “more pleasantly active and more easy to handle during therapy” by the father, thereby prompting his request for resumption of the trial. No clinical or biochemical side effects were observed, which suggests that higher doses may be administered with additional beneficial effects.

With respect to mast cell degranulation, treatment with, for example, AICA riboside or ribavirin will benefit patients suffering from a variety of illnesses. For example, individuals suffering from allergies, particularly asthma, hay fever (including allergic conjunctivitis and allergic rhinitis), chronic urticaria, urticaria pigmentosa and eczema, can be expected to benefit from purine nucleoside and purine nucleoside analog treatment. As discussed in B. Benacerra and A. Unanue in Textbook of Immunology (Williams & Williams Baltimore/London, 1979), a key to suppressing allergic responses is to prevent the release of pharmacologically active substances by mast cells. Mast cells are large basophilic staining cells with extensive granules that contain substances, such as histamines, that are liberated by the mast cell during an allergic reaction and are required to support the allergic response. The release of these pharmacologically active substances present in mast cells is termed “degranulation.” Thus, chemicals that prevent degranulation should have a beneficial effect on reducing the severity of the allergic response. As such, patients experiencing allergies can be successfully treated with AICA riboside or ribavirin, as these molecules prevent mast cell degranulation. Mast cell activation also causes the release of prostaglandins and leukotrienses (non-preformed mediators) such as slow reactive substance of anaphylaxis. The purine nucleosides and analogs useful in the invention also prevent release of these mediators of inflammation.

Applicant's invention relates to the discovery of particularly therapeutive administration of concentrations of AICA riboside for the prevention of tissue damage associated with decreased blood flow in humans, and the determination of dosages which achieve efficacy while avoiding undesirable side effects. Applicant's invention also relates to the discovery of particularly therapeutic concentrations and dosages of AICA riboside which prevent or reduce the severity of adverse clinical outcomes, including adverse cardiovascular and/or cerebrovascular events in patients at risk for such events. Applicant has discovered that it is preferable to maintain an intravascular concentration of AICA riboside of from about 1 μg/ml to about 20 μg/ml, to obtain the beneficial effects of AICA riboside, and to prevent side effects which may occur at higher dosages. Applicant has discovered that the ideal range is about 3 to about 6 μg/ml, and especially about 5 μg/ml.

Thus, in a first aspect, the invention features a method of preventing tissue damage associated with decreased blood flow in humans by administering AICA riboside to a person in an amount, which maintains a blood plasma concentration of AICA riboside for a sufficient time so that the risk of such tissue damage is reduced in that person, of from about 1 μg/ml to about 20 μg/ml, preferably a concentration of about 3 μg/ml to about 6 μg/ml, and more preferably at about 5 μg/ml. It is desirable that the concentration of AICA riboside in the person results in an elevation of serum uric acid to a level of no greater than about 16.0 mg/dl, and more preferably no greater than about 9.0 mg/dl.

By “preventing tissue damage” is meant lessening the frequency, duration and/or severity of ischemic events and/or reducing the deleterious effects of undesired decreased blood flow on the tissue. The incidence, duration and severity of ischemic events may be measured by methods known in the art. For example, in the use of AICA riboside during coronary artery bypass graft (CABG) surgery, the following methods may be employed: (1) comparison of ST segment changes on continuous Holter electrocardiographic recordings; (2) assessment of regional wall motion by transesophageal echocardiography; (3) serial measurement of creatinine phosphokinase MB; and (4) serial 12-lead electrocardiographic analyses. Methods for measurement of deleterious effects of undesired decreased blood flow are also known in the art. Deleterious effects of tissue damage may include adverse clinical outcomes, such as adverse cardiovascular and/or cerebrovascular events including those observed in connection with CABG surgery. Such adverse events include cardiac death (i.e., death due to primarily a heart-related cause), transmural and/or non-transmural myocardial infarction, cerebrovascular accident, congestive heart failure, and life-threatening dysrhythmia, which may occur during and/or following such surgery. Other adverse clinical outcomes which may be prevented by administration of AICA riboside include hepatic injury (documented by enzyme elevation), pancreatic injury (documented by enzyme elevation), disseminated intravascular coagulation (including that due to bowel ischemia) and death (from non-cardiac causes). By reducing the risk of tissue damage is meant diminishing the opportunity for tissue damage as compared to the opportunity which existed without the administration of AICA riboside. The use of AICA riboside or prodrugs thereof may also protect brain tissue from injury due to decreased blood flow.

By AICA riboside is meant 5-amino-1-β-D-ribofuranosyl-imidazole-4-carboxamide (also known as acadesine).

In a second aspect, the invention features a method of preventing tissue damage associated with decreased blood flow in humans by administering AICA riboside for a sufficient time to reduce the risk of such tissue damage, in a dosage of from about 0.01 mg/kg/min to about 2.0 mg/kg/min; preferably, from about 0.05 mg/kg/min to about 0.2 mg/kg/min; and more preferably of about 0.1 mg/kg/min for anesthetized patients and about 0.125 mg/kg/min for non-anesthetized patients or those patients anesthetized for a short period of time.

In certain embodiments, the tissue to which damage is prevented is cardiac muscle or cardiac microvasculature. In other embodiments, the tissue to which damage is prevented is brain tissue or brain microvasculature.

In certain embodiments, the tissue damage which is prevented is that tissue damage which occurs as a result of undesired decreased blood flow occurring during surgery, such as during cardiac surgery (for example, CABG surgery) or during vascular surgery. In these embodiments, the compound may be administered beginning shortly before the induction of anesthesia, and continue through the duration of the surgery, and for about one hour following completion of surgery, or for at least about seven hours following completion of surgery, or longer, depending on factors such as duration of the surgery.

In another embodiment, the AICA riboside is administered both to a patient undergoing cardiac surgery and in the perfusate solution used to perfuse the patient's heart during such surgery. Preferably, the AICA riboside concentration in the perfusate solution is in the range of about 5 μM to about 100 μM, more preferably about 20 μM.

In another embodiment, AICA riboside is administered in combination or conjunction with allopurinol, preferably in an amount of between about 100 mg/day to about 1200 mg/day, and more preferably in an amount of about 300 mg/day. Allopurinol reduces uric acid levels and thus, may be administered in combination with, or in conjunction with, AICA riboside (or a prodrug of AICA riboside) to allow administration of a larger dosage of AICA riboside or prodrug while avoiding adverse side effects of increased uric acid levels. As noted above, it is desirable for uric acid levels not to exceed about 16 mg/dl, and preferable for them not to exceed about 9 mg/dl.

In another embodiment, the invention further involves the identification of a person in need of prevention of such decreased blood flow, prior to administering AICA riboside (or a prodrug thereof). Those skilled in the art will recognize that by “identification” is meant determination of patients at risk for tissue damage, e.g., those patients undergoing surgery or other procedures. Risk factors for those patients undergoing cardiac surgery include elevated age (for example, above 70 years of age); emergency or urgent surgery, which may be complicated by unstable angina; failed percutaneous transluminal coronary angioplasty; decreased left ventricular function (as determined by an ejection fraction of less than about 40%); chronic or acute renal failure; dysrhythmia (under treatment); or MI within the past several years. See, e.g., Mangano, Anesthesiology 72:153-184 (1990). Risk factors for those patients undergoing non-cardiac surgery include elevated age (for example, above 65-70 years of age); atherosclerotic heart disease, i.e., coronary artery disease, as evidenced by peripheral vascular disease or carotid artery disease; diabetes; renal failure; heart failure currently under therapy; left ventricular hypertrophy and hypertension; hypertension for over 5 years; emergency or urgent surgery; MI within 6 months to a year prior to surgery; angina; arrhythmia or hypercholesterolemia. The invention also includes identification of patients who are in need of prophylactic administration of AICA riboside because of a chronic, genetic, or similar condition, or due to angina, transient ischemic attack, evolving or recent MI, or evolving or recent stroke. Thus, those not undergoing surgery may face an increased risk for tissue damage, as well.

In another aspect, the invention features a method of preventing tissue damage associated with decreased blood flow in a human by administering a total dose of AICA riboside in an amount of from 10 mg/kg to 200 mg/kg; preferably in an amount between 30 mg/kg and 160 mg/kg. For cardiac surgery, a preferred amount is about 40 mg/kg. For other indications, such as non-cardiac surgery, a preferred amount is about 120 mg/kg. Those skilled in the art will recognize that such total doses can be achieved by varying the concentration of AICA riboside administered, the rate of administration and/or the duration of administration.

In another aspect, the invention features a method of prevention of tissue damage associated with undesired decreased blood flow in humans by administering a prodrug of AICA riboside in an amount effective to provide a blood plasma level of AICA riboside from about 1 μg/ml to about 20 μg/ml, preferably about 3 μg/ml to about 6 μ/ml and more preferably about 5 μg/ml. The amount of prodrug necessary to achieve these levels is readily determined by one skilled in the art using standard methodologies. A prodrug may be administered in combination with, or in conjunction with, allopurinol, preferably with allopurinol being administered in an amount of from about 100 mg/day to about 1200 mg/day, and preferably in an amount of about 300 mg/day. Such administration will avoid adverse side effects of high uric acid levels. A prodrug may be administered as described above for AICA riboside itself.

In another aspect, the invention features a method of preventing adverse clinical outcomes, including adverse cardiovascular and/or cerebrovascular events, in those at risk for such outcomes, which comprises administering AICA riboside, or a prodrug thereof, in an amount which provides a blood plasma concentration of AICA riboside of between about 1 μg/ml and about 20 μg/ml, preferably between about 3 μg/ml and about 6 μg/ml and more preferably about 5 μg/ml. By “adverse clinical outcome” is meant an event which has a clinically detrimental effect on a patient. By “adverse cardiovascular event” is meant an event pertaining to the heart or blood vessels which is detrimental to a patient. By “adverse cerebrovascular event” is meant an event pertaining to blood vessels affecting the brain which is detrimental to a patient.

The invention further involves the identification of patients at risk for adverse clinical outcomes, including adverse cardiovascular and adverse cerebrovascular events. Risk factors for those patients undergoing cardiac surgery include elevated age (for example, above 70 years of age); emergency or urgent surgery, which may be complicated by unstable angina; failed percutaneous transluminal coronary angioplasty; decreased left ventricular function (as determined by an ejection fraction of less than about 40%); chronic or acute renal failure; dysrhythmia (under treatment); or MI within the past several years. See, e.g., Mangano, Anesthesiology 72:153-184 (1990). Risk factors for those patients undergoing non-cardiac surgery include elevated age (for example, above 65-70 years of age); atherosclerotic heart disease, i.e., coronary artery disease, as evidenced by peripheral vascular disease or carotid artery disease; diabetes; renal failure; heart failure under therapy; left ventricular hypertrophy and hypertension; hypertension for over 5 years; emergency or urgent surgery; MI within 6 months to a year prior to surgery; angina; arrhythmia or hypercholesterolemia. The invention also includes identification of patients who are in need of prophylactic administration of AICA riboside because of a chronic, genetic, or similar condition, or due to angina, transient ischemic attack, evolving or recent MI, or evolving or recent stroke. Thus, those not undergoing surgery may face an increased risk for tissue damage, as well.

In another aspect, the invention features a method of preventing adverse clinical outcomes, including adverse cardiovascular and/or cerebrovascular events in those at risk for such events by administering AICA riboside for a sufficient time to reduce the risk of such events, in a dosage of from about 0.01 mg/kg/min to about 2.0 mg/kg/min; preferably from about 0.05 mg/kg/min to about 0.2 mg/kg/min; and more preferably of about 0.1 mg/kg/min or 0.125 mg/kg/min, depending on anesthesia.

In certain embodiments, the adverse cardiovascular event which is prevented is myocardial infarction. “Myocardial infarction” includes transmural and non-transmural myocardial infarction. In the case of CABG surgery, transmural MI is evidenced by the presence of a new Q wave in ECG testing and an elevated CK-MB concentration, and non-transmural MT is evidenced by elevated CK-MB concentration without a new Q wave. In other embodiments, the cardiovascular event which is prevented is cardiac death. By “cardiac death” is meant death of a patient from a primary cardiac cause, for example, from myocardial infarction, dysrhythmia or ventricle dysfunction.

Another aspect of the invention provides a method of preventing or reducing adverse effects in a patient who has had a myocardial infarction by administering an effective amount of acadesine, or a prodrug, analog, or salt thereof. In one embodiment, the myocardial infarction occurred within the last 24, 36, or 48 hours. Another embodiment provides a method where the patient is female and/or or between the age of 65 and 95.

In certain embodiments, the cerebrovascular event which is prevented is cerebrovascular accident. By “cerebrovascular accident” is meant injury to the brain associated with decreased blood flow, e.g., stroke.

In certain embodiments, the risk for adverse cardiovascular or cerebrovascular event occurs as a result of indications such as angina or transient ischemic attack. In other embodiments, the risk of adverse cardiovascular or cerebrovascular events occurs as a result of cardiac surgery, for example, CABG surgery, or as a result of non-cardiac surgery, for example, vascular surgery. In the case of surgery, the AICA riboside may be administered beginning shortly before the induction of anesthesia, and continued through the duration of surgery, for about 1 hour following completion of surgery, or for about 7 hours total. Administration may continue for a longer time, for example, 24 hours or more following surgery. Prolonged administration is especially effective for non-cardiac surgery because adverse events tend to occur later. For example, it has been observed that in cardiac surgery, MI tends to occur mainly in the first day following surgery, however, in non-cardiac surgery, MI tends to occur mainly in the second or third day following surgery. Thus, in the case of non-cardiac surgery, AICA riboside (or a prodrug) is administered for a more prolonged period after surgery, for example, for 7-48 hours.

Another aspect of the invention provides a method of preventing or reducing adverse effects in a patient undergoing non-vascular surgery by administering an effective amount of acadesine, or a prodrug, analog, or salt thereof. Non-vascular surgery includes abdominal, neurological, gynecological, orthopedic, urological, and otolaryngological surgery. More specifically, the non-vascular surgery includes, small and large bowel resection, appendectomy, laparoscopy, paracentesis, transurethral resection of the prostate (TURP), hysterectomy, tuba ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, liposuction and the like.

In another embodiment, the AICA riboside is administered both to a patient undergoing cardiac surgery, and in the perfusate solution used to perfuse the patient's heart during such surgery. Preferably, the AICA riboside concentration in the perfusate solution is in the range of about 5 μM to about 100 μM, more preferably about 20 μM.

In another embodiment, AICA riboside is administered in combination or conjunction with allopurinol, preferably in an amount of between about 100 mg/day and about 1200 mg/day, and more preferably in an amount of between about 300 mg/day.

In another embodiment, the invention provides a method for preventing or reducing the occurrence of an adverse cardiovascular or cerebrovascular event in a patient undergoing CABG surgery, which method comprises the steps of: (a) administering to said patient 0.1 mg/kg/min AICA riboside intravenously for about 7 hours perioperatively; and (b) perfusing the heart of said patient with a perfusate solution of 20 μM AICA riboside.

Yet another aspect of the invention provides a method of preventing stroke in a patient undergoing CABG by administering an effective amount of acadesine, or a prodrug, analog, or salt thereof.

In another aspect, the invention features a method of preventing, or reducing the severity of, myocardial infarction in a human at risk for myocardial infarction, which method comprises administering AICA riboside or a prodrug thereof to said human in an amount which provides a blood plasma concentration of AICA riboside in said human of between about 3 μg/ml and about 6 μg/ml, for a sufficient time to reduce the risk of said myocardial infarction. Increased risk of myocardial infarction may result from surgery, either cardiac surgery, such as CABG surgery, or non-cardiac surgery, such as vascular surgery, or from factors other than surgery, e.g., indications of reversible ischemia, such as angina or silent ischemia, or of evolving or recent MI or stroke.

In another aspect, the invention features a method of preventing, or reducing the severity of, cerebrovascular accident in a human at risk for cerebrovascular accident, which method comprises administering AICA riboside or a prodrug thereof to said human in an amount which provides a blood plasma concentration of AICA riboside in said human of between about 3 μg/ml and about 6 μg/ml, for a sufficient time to reduce the risk of said cerebrovascular accident. Increased risk of cerebrovascular accident may result from surgery, either cardiac (such as CABG surgery) or non-cardiac (such as vascular surgery) or from non-surgical risks such as transient ischemic attack.

In another embodiment, the invention features a method of preventing, or reducing the severity of, cardiac death, which method comprises administering AICA riboside or a prodrug thereof to said human in an amount which provides a blood plasma concentration of AICA riboside in said human of between about 3 μg/ml and about 6 μg/ml, preferably about 5 mg/ml, for a sufficient time to reduce the risk of said cardiac death. Increased risk of cardiac death may result from the surgery, cardiac or non-cardiac. For example, the risk may result from CABG surgery.

The AICA riboside may be administered continuously or in a plurality of doses. To reduce the risk of tissue damage, the AICA riboside may be administered for a period of at least about 15 minutes. It may be administered for a duration of greater than about 4 hours and preferably for a duration of about 7 hours. In other cases, the AICA riboside may be administered for a duration of greater than about 10, 12, 16, 24, or even about 48 hours.

The AICA riboside may be administered intravenously, by intracoronary or intraarterial infusion, orally, or by any other methods known in the art, including introduction into the patient's blood in an extracorporeal circuit, for example, using a heart-lung machine or dialysis. AICA riboside may be administered prophylactically, or in response to a known bodily condition.

In one embodiment, AICA riboside is prepared as a therapeutic solution from a lyophilized form to prevent variable discoloration of a liquid formulation observable during storage. Preferably, the AICA riboside is non-pyrogenic.

Another aspect provides a pharmaceutical formulation comprising acadesine, or a prodrug, analog, or salt thereof and a pharmaceutically acceptable carrier, diluent or excipient, wherein the formulation provides a patient in need with a blood plasma concentration of acadesine, or a prodrug, analog, or salt thereof between about 1 μg/ml to about 20 μg/ml for a sufficient amount of time, and the formulation is lipophilic. In one embodiment, the amount of time is about seven hours. In another embodiment, the pharmaceutical formulation is in a micelle form.

In another aspect, the invention features a kit for use in administering AICA riboside to a patient undergoing cardiac surgery, e.g., CABG surgery, which comprises lyophilized AICA riboside for use in preparing an AICA riboside solution for intravenous infusion into a patient undergoing cardiac surgery and AICA riboside in solution for use in preparing a cardioplegic perfusate solution to be used to perfuse the heart of a patient undergoing cardiac surgery. Preferably, the AICA riboside is non-pyrogenic. Preferably, the lyophilized AICA riboside is provided in an amount of from 100 mg to 2,000 mg; more preferably in an amount of 500 mg. Preferably, the AICA riboside in solution is provided in a volume of from 1 ml to 20 ml; more preferably 5 ml. Preferably, the concentration of the AICA riboside in solution is about 1 mg/ml.

The lyophilized AICA riboside may be combined with a suitable diluent, such as water or saline solution to put it in a form suitable for infusion into the patient.

The AICA riboside in solution may be in a solution of water, saline solution, or cardioplegic solution. The AICA riboside in solution is of a concentration suitable for adding to cardioplegic perfusate solution such that the final concentration of AICA riboside in the cardioplegic solution is from 5 μM to 100 μM, preferably 20 μM. For example, if 5 ml of 1 mg/ml AICA riboside is added to one liter of cardioplegic perfusate solution, the resulting concentrate will be approximately 5 μg/ml or 20 μM.

One of the advantages of applicant's discovery of the particularly useful therapeutic concentrations and dosages of AICA riboside is that efficacy can be obtained at dosages at which the side effects of elevated serum or urinary uric acid levels and/or crystalluria are lessened, if not avoided altogether, and which avoid decreased blood glucose levels.

Applicant also discovered that lower doses of AICA riboside were needed to achieve the desired blood concentration levels in anesthetized patients than in non-anesthetized patients. It appears that the dose needed in anesthetized patients may be about 20-50% less than the dose needed in non-anesthetized patients. Thus, a preferred dosage of AICA riboside (or prodrug) in a non-anesthetized patient or a patient anesthetized for a short time is larger than the preferred dosage for an anesthetized patient. Accordingly, dosages of from about 0.075 mg/kg/min to about 0.30 mg/kg/min are preferred in such cases, more preferably between about 0.10 mg/kg/min and about 0.15 mg/kg/min, and most preferably about 0.125 mg/kg/min.

### DEFINITIONS

As used herein, the following terms have the following meanings, unless expressly stated to the contrary.

The term “hydrocarbyl” refers to an organic radical comprised of primarily carbon and hydrogen and includes alkyl, alkenyl and alkynyl groups, as well as aromatic groups including aryl and aralkyl groups and groups which have a mixture of saturated and unsaturated bonds, alicyclic (carbocyclic or cycloalkyl) groups or such groups substituted with aryl (aromatic) groups or combinations thereof and may refer to straight-chain, branched-chain or cyclic structures or to radicals having a combination thereof.

The term “alkyl” refers to saturated aliphatic groups, including straight, branched and carbocyclic groups. The term “lower alkyl” refers to both straight- and branched-chain alkyl groups having a total of from 1 to 6 carbon atoms and includes primary, secondary and tertiary alkyl groups. Typical lower alkyls include, for example, methyl, ethyl, n-propyl, isopropyl, n-butyl, isobutyl, t-butyl, n-pentyl, n-hexyl, and the like.

The term “aryl” refers to aromatic groups having from about 6 to 14 carbon atoms and includes cyclic aromatic systems such as phenyl and naphthyl.

The term “aralkyl” refers to an alkyl group of about 1 to 4 carbon atoms substituted with an aryl group of from 6 to 10 carbon atoms and includes, for example, benzyl, p-chlorobenzyl, p-methylbenzyl and 2-phenylethyl.

The term “alkenyl” refers to unsaturated alkyl groups having at least one double bond>e.g. CH₃ CH═CH(CH₂)₂— and includes both straight and branched-chain alkenyl groups.

The term “alkynyl” refers to unsaturated groups having at least one triple bond>e.g. CH₃C≡C(CH₂)₂—! and includes both straight chain and branched-chain groups.

The term “halo” or “halogen” refers to fluorine, chlorine, bromine and iodine.

The term “acyl” refers to the group

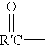

wherein R¹ is hydrocarbyl.

The term “alkylene” refers to straight, branched-chain and carbocyclic alkylene groups which are biradicals, and includes, for example, groups such as ethylene, propylene, 2-methylpropylene (e.g.

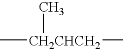

1,6-n-hexylene, 3-methylpentylene (e.g.

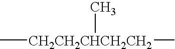

1,4-cyclohexylene, and the like.

The term “amide” or “amido” refers to the group

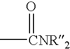

wherein each R″ is independently hydrogen or hydrocarbyl, or to compounds having at least one such group.

The term “carboxamide” refers to the group

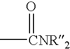

wherein each R″ is independently hydrogen or hydrocarbyl. The term “unsubstituted carboxamide” refers to the group

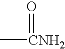

The term “acylamino” refers to the group

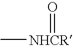

wherein R′ is hydrocarbyl. The term “lower acylamino” refers to acylamino groups wherein R′ is alkyl of 1 to 6 carbon atoms.

The term “carbonate ester” refers to the group

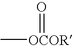

wherein R′ is hydrocarbyl or to compounds having at least one such group.

The term “acyl ester” refers to the group

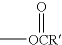

wherein R′ is hydrocarbyl or to compounds having at least one such group.

The term “phosphate ester” refers to the group

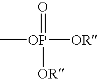

wherein R″ is independently hydrogen or hydrocarbyl and/or to compounds having at least one such group, and includes salts thereof.

The term “mixed ester” refers to compounds having at least one carbonate ester group and at least one acyl ester group or to compounds having combinations of different acyl ester or carbonate ester groups.

The term “carboxylic acid ester” or “carboxy ester” refers to the group

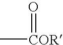

wherein R′ is hydrocarbyl or to compounds having at least one such group.

The term “carbocyclic AICA riboside” refers to an analog of AICA riboside wherein the oxygen atom in the ribosyl ring has been replaced by a methylene (—CH₂—).

The term “hydrocarbyloxy” refers to the group R′O— wherein R′ is hydrocarbyl.

The term “alkoxy” refers to the group R′O— wherein R′ is alkyl.

The term “hydrocarbylthio” refers to the group having the formula R′O— wherein R′ is hydrocarbyl.

The term “hydrocarbylamino” refers to the groups —NHR′ or —NR′₂ where R′ is an independently selected hydrocarbyl group.

The term “hydrocarbylimidate” refers to the group

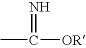

wherein R″ is hydrocarbyl.

The term “carboxamideoxime” refers to the group

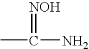

The term “hydrocarbyloxyamidine” refers to the group

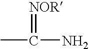

wherein R′ is hydrocarbyl.

The term “hydrocarbyloxycarbonyl refers to the group

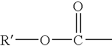

wherein R′ is hydrocarbyl.

The term “hydrocarbyloxycarboxy” refers to the group

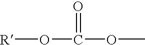

wherein R′ is hydrocarbyl.

The term “thioester” refers to the group

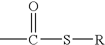

wherein R′ is hydrocarbyl.

Preferred AICA Riboside Analogs

According to the present invention, preferred analogs of AICA riboside include compounds of the formula I

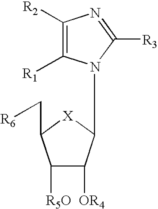

or a pharmaceutically acceptable salt thereof wherein X is —O— or —CH₂—; R₁ is hydrogen, amino, hydrocarbylamino, acylamino, or dihydrocarbylaminoalkyleneimino; R₂ is hydrogen, cyano, hydrocarbylimidate, carboxamideoxime, hydrocarbyloxyamidine, carboxamide, or carboxylic acid or an amide, ester, thioester or salt thereof; R₃ is hydrogen, hydrocarbyl, amino, hydrocarbylamino, halogen, hydroxy (including tautomeric 2-imidazolone), hydrocarbyloxy, sulfhydryl (including tautomeric 2-imidazolthione), or hydrocarbylthio; R₄ and R₅ are independently hydrogen, alkyl, acyl or hydrocarbyloxycarbonyl; R₆ is hydrogen, hydrocarbyl, halogen, hydroxy, hydrocarbyloxy, sulfhydryl, hydrocarbylthio, sulfamyloxy, amino, hydrocarbylamino, azido, acyloxy or hydrocarbyloxycarboxy or phosphate ester group or salts thereof; provided that when R₁ is amino, R₂ is unsubstituted carboxamide, R₃ is hydrogen; R₄ and R₅ are hydrogen, acyl or hydrocarboxycarbonyl; then R₆ is not hydroxy, acyloxy or hydrocarbyloxycarboxy.

Alternatively R₂ may be a group of the formula:

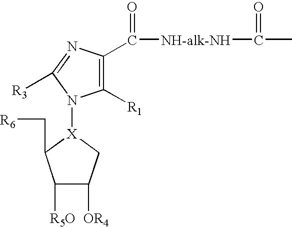

wherein R₁, R₃, R₄, and R₅ and R₆ are as previously
defined in conjunction with formula (I) and alk is an alkylene group of from 2 to 8 carbon atoms. Suitable alk groups include n-hexylene and 1,4-cyclohexylene. Since compounds of the above formula wherein R₃ is hydroxy or sulfhydryl may exist in their isomeric (tautomeric)imidazole-2-one and imidazole-2-thione forms, these isomers are intended to be included in the ambit of Formula I.

Preferred compounds include those wherein (i) R₁ is amino, R₂ is carboxamide wherein one of the amide hydrogens is replaced by a hydrocarbyl group, more preferably an aralkyl group (such hydrocarbyl or aralkyl group is optionally substituted, suitable substituents include those set forth below); R₃ is hydrogen, R₄ and R₅ are hydrogen or hydrocarbyloxycarbonyl, more preferably and R₆ is hydroxy or amino (Series I); (ii) R₁ is amino, R₂ is carboxamide, R₃ is halogen or sulfhydryl, R₄ is hydrogen, R₅ is hydrogen and R₆ is hydroxy (Series II); (iii) R₁ is amino, R₂ is carboxamide; R₃, R₄ and R₅ are hydrogen and R₆ is amino (Series III) and (iv) R₁ is amino, R₂ is carboxamide, R₃ is hydrogen, R₄ is alkyl, R₅ is hydrogen and R₆ is hydroxy (Series IV).

In particular, in view of their demonstration of activity in various experimental models, preferred compounds include Compound Nos. 10, 23, 25, 29, 47, 52, 53 (Series I), 27, 43 (Series II), 21, 66 (Series III) and 20, 34 (GP-1-250) and 32 (GP-1-262) (Series IV) of Tables XII and XIII.

Preferred Novel AICA Riboside Analogs

One preferred group of compounds of formula I include certain novel AICA riboside analogs wherein X is —O— or —CH₂—; R₁ is amino, hydrocarbylamino or dihydrocarbylaminoalkyleneimino, R₂ is carboxamide wherein one of the amide hydrogens (attached to the nitrogen atom) is optionally replaced by alkyl, cycloalkyl, or aryl or aralkyl, optionally substituted with 1 to 3 substituents independently selected from halogen, alkyl, aryl, nitro, amino, hydrocarbylamino, sulfhydryl, hydrocarbylthio, hydroxy, hydrocarbyloxy, trifluoromethyl, or sulfonamide; R₂ is carboxamide wherein both amide hydrogens are replaced by alkyl or together by an alkylene or aralkylene group to form a ring; or R₂ is —C(O)—S—R₇ wherein R₇ is alkyl, cycloalkyl, aryl or aralkyl optionally substituted with 1 to 3 substituents independently selected from halogen, alkyl, aryl, nitro, amino, hydrocarbylamino, sulfhydryl, hydrocarbylthio, hydroxy, hydrocarbyloxy, trifluoromethyl or sulfonamide; or further, R₂ is a group of formula II wherein R₁, R₃, R₄, R₅ and R₆ are as defined with formula I and alk is alkylene of 2 to 8 carbon atoms; R₃ is hydrogen, amino, hydrocarbylamino, halogen, hydroxy (including tautomeric imidazolone), hydrocarbyl, sulfhydryl (including tautomeric 2-imidazolthione) or hydrocarbylthio; R₄ and R₅ are independently hydrogen, hydrocarbyl (of 1 to about 18 carbon atoms), acyl or hydrocarbyloxycarbonyl; and R₆ is hydroxy, hydrogen, hydrocarbyl, halogen, hydrocarbyloxy, sulfhydryl, hydrocarbylthio, sulfamyloxy, amino, hydrocarbylamino, azido, acyloxy, hydrocarbyloxycarboxy or phosphate ester or salt thereof; provided that when —X— is —O— or —CH₂—, R₁ is amino, R₂ is unsubstituted carboxamide, R₃ is hydrogen, R₄ and R₅ independently are hydrogen, acyl or hydrocarbyloxycarbonyl, then R₆ is not hydrogen, hydroxy, acyloxy or hydrocarbyloxycarboxy or when R₄ and R₅ are both hydrogen, then R₆ is not a phosphate ester; when X is oxygen, R₁ is amino, R₂ is unsubstituted carboxamide, R₃ is sulfhydryl, and R₄ and R₅ are both hydrogen, then R₆ is not acetoxy; when X is oxygen, R₁ is amino, R₂ is unsubstituted carboxamide and R₃ is chloro, bromo, amino or methoxy, and R₄ and R₅ both hydrogen, then R₆ is not hydroxy or when R₄ and R₅ are both acetyl, then R₆ is not acetoxy; and provided further that when X is oxygen, R₁ is amino, R₂ is benzylcarboxamide or p-iodophenylcarboxamide, R₃ is hydrogen, then R₄ and R₅ are not both hydrogen and R₆ is not hydroxy; or when R₂ is p-iodophenylcarboxamide, then R₄ and R₅ are not both acetyl and R₆ is not acetoxy.

Preferred compounds include those wherein R₁ is amino, R₂ is carboxamide substituted with an aralkyl group, more preferably a benzyl group, having from 1 to 3 ring substitutions as described above, or cycloalkyl. In view of their activity in various experimental models, preferred compounds include Compound Nos. 23, 25, 29, 47, 52 and 53.

One example of an especially preferred compound is a compound where X is oxygen, R₁ is amino, R₂ is p-chlorobenzylcarboxamide, R₃, R₄ and R₅ are hydrogen and R₆ is amino and salts thereof. One particularly preferred salt is the hydrochloride salt. Other particularly preferred salts are sodium and potassium salts, especially disodium and mono potassium.

A preferred AICA riboside analog is a compound represented by the formula (Ia)

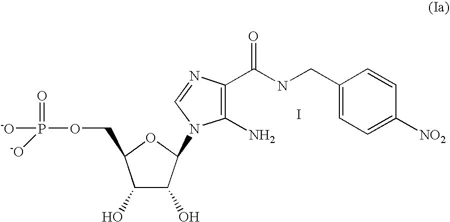

In one embodiment, the invention provides a prodrug, analog, or salt of the compound of formula Ia.

In one aspect, the invention provides a kit for use in administering the compound of formula Ia, or a prodrug, analog, or salt thereof to a patient undergoing cardiac surgery comprising a lyophilized form of the compound of formula Ia, or a prodrug, analog, or salt thereof for infusion into the patient and the compound of formula Ia, or a prodrug, analog, or salt thereof in solution for perfusion into the heart of the patient. In another aspect, the invention provides a kit for administering the compound of formula Ia, a prodrug, analog, or salt thereof to a patient comprising a sterile container of lyophilized compound represented by formula Ia, or a prodrug, analog, or salt thereof.

In one embodiment, the invention provides a cardioplegic solution comprising a composition comprising the compound of formula Ia.

In another embodiment, the invention provides a method of preventing or reducing adverse effects in a patient undergoing CABG surgery comprising administering perioperatively to the patient an effective amount of a composition comprising the compound of formula Ia, or a prodrug, analog, or salt thereof.

In another embodiment, the invention provides a pharmaceutical formulation comprising the compound of formula Ia, or a prodrug, analog, or salt thereof and at least one pharmaceutically acceptable carrier, diluent, or excipient. In one embodiment, the formulation provides a patient in need thereof with a blood plasma concentration of between about 1 μg/ml and about 20 μg/ml over a sufficient period of time. In another embodiment the period of time is about seven hours. In another embodiment, the formulation is adapted for oral administration. In another embodiment, the formulation is adapted for oral administration in a solid dosage form.

In another aspect, the invention provides a cardioplegic solution comprising a composition comprising a compound represented by the formula (IIa)

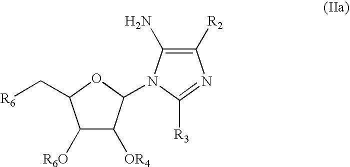

wherein R₂ is selected from the group consisting of hydrogen, —CN and the group

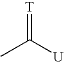

where T is selected from oxygen, sulfur, NOH, NH, and NO(CH₂)ₙCH₃ where n is from 0 to 2) and U is selected from lower alkoxy, amino, a 3 to 6 member heterocyclic ring optionally fused to a 3 to 6 member aryl ring, and the group

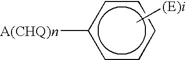

wherein A is one of NH and S, n is from 0 to 3, i is from 0 to 2, Q is one of hydrogen and hydroxy, and E represents a nitro or hydroxy group, provided that where U is amino, T is not one of sulfur, NOH, NH, and NOCH₃; where T is amino, U is not lower alkoxy; and where A is amino and n is 1, Q is not hydroxy;
R₃ is selected from hydrogen, halogen, and S—W, where W is phenyl, or substituted phenyl, or hydrogen when T is not oxygen and U is not amino;
R₄ and R₅ are each independently selected from hydrogen, —COCH₃ and lower alkyl, or together form a cyclic carbonate; and
R₆ is selected from, hydroxy, phosphate ester, —OSO₂ NH₂, sulfhydryl, halogen, —OCOCH₃, —SCH₃, —SOCH₃, NH₂ and N₃; and pharmaceutically acceptable salts thereof;
provided that when R₂ is CONH₂, CONH-para-iodophenyl, hydrogen, CN, or CONHCH₂-φ and R₃ is hydrogen or halogen, and R₄ and R₅ are hydrogen, acyl, or together form a cyclic carbonate, then R₆ is not halogen, phosphate ester, OH, or —O-acyl wherein said compound, a prodrug, analaog, or salt thereof is at a concentration of between 5 μM to 100 μM.

In one embodiment, a kit for use in administering an acadesine analog to a patient undergoing cardiac surgery may comprise a lyophilized form of the compound of formula Ia or IIa, a prodrug, analog, or salt thereof for infusion into the patient and/or a solution form of the compound of formula Ia or Ia, a prodrug, analog, or salt thereof for perfusion into the heart of the patient.

A preferred AICA riboside analog is 5-amino-1-β-D-(5-benzylamino-5-deoxy-1-β-D-ribofuranosyl)imidazole-4-carboxamide, having the chemical structure of formula (IIIa)

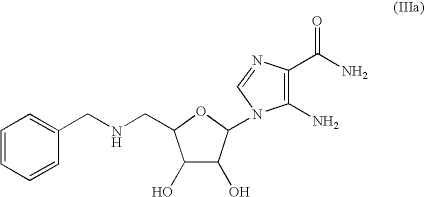

Another preferred AICA riboside analog is 5-amino-1-(5-amino-5-deoxy-β-D-ribofuranosyl)imidazole-4-N-[(4-chlorophenyl)methyl]carboximide having the chemical structure of formula (IVa):

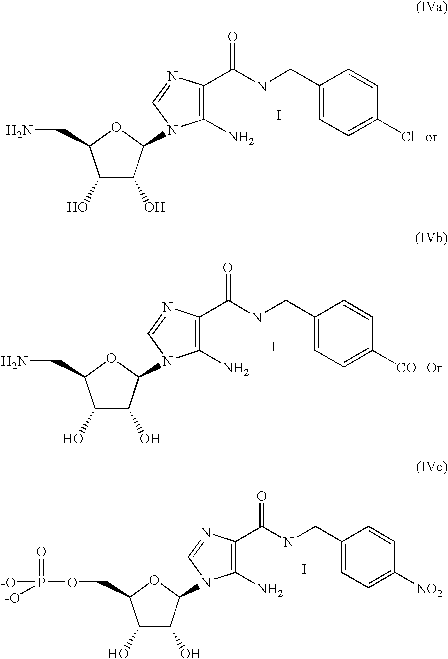

In one embodiment, the prodrug, analog, or salt of compound of formula IIIa, formula IVa, formula IVb or formula IVc is provided. In a further embodiment, such compounds are provided in a patch drug delivery system. In a yet further embodiment, at least one or more of such compounds is provided along with a secondary compounds that are utilized to enhance penetration, effect stabilization or have anti-irritant characteristics.

In another aspect, the invention provides a method of preventing or reducing adverse effects in a patient with decreased left ventricular function having an ejection fractoin that is about less than 30% comprising administering to the patient an effective amount of the compound of formula Ia, IIa, IIIa or formula IVa, or a prodrug, analog, or salt thereof. In another aspect, the invention provides a method of preventing for reducing adverse effects in a patient by administering an effective amount of the compound of formula Ia, Ia, IIIa or formula IVa, a prodrug, analog, or salt thereof, wherein the patient has had one, two, three, or more than three past myocardial infarctions. In one embodiment, the most recent myocardial infarction occurred within the last 24, 36 or 48 months. In another embodiment of the two methods described above, the patient is female and/or is between the age of 65 and 95. In another embodiment, the compound of formula Ia, Ia, IIIa or IVa, or a prodrug, analog, or salt thereof is administered at a concentration which provides a blood plasma concentration in a patient of between about 1 μg/ml to about 20 μg/ml over a sufficient period of time. In one embodiment, the blood plasma concentration is maintained over a period of about seven hours. In another embodiment, the compound of formula IIIa or formula IVa, or a prodrug, analog, or salt thereof is administered at 0.1 mg/kg/minute. In another embodiment, the compound of formula IIIa or formula IVa is administered to a patient over about seven hours.

Another aspect of the invention provides a method of preventing or reducing adverse effects in a patient undergoing a non-vascular surgery comprising administering to the patient an effective amount of the compound of formula Ia, Ia, IIIa or formula IVa, or a prodrug, analog, or salt thereof. In one embodiment, the invention can be used on a wide variety of non-vascular surgeries, including, but not limited to, cardiac, abdominal, neurological, gynecological, orthopedic, urological, vascular, and surgery related to otolaryngology. More specifically, non-vascular surgery includes, small and large bowel resection, appendectomy, laparoscopy, paracentesis, transurethral resection of the prostate (TURP), hysterectomy, tuba ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, liposuction and the like.

Another aspect provides a pharmaceutical formulation comprising a compound of formula IIIa or formula IVa, or a prodrug, analog, or salt thereof and a pharmaceutically acceptable carrier, diluent or excipient, wherein the formulation provides a patient in need with a blood plasma concentration of a compound of formula IIIa or formula IVa, or a prodrug, analog, or salt thereof between about 1 μg/ml to about 20 μg/ml for a sufficient amount of time. In another embodiment, the amount of time is about seven hours. In another embodiment, the pharmaceutical formulation is in a micelle form. In one embodiment, the formulation is lipophilic.

In another aspect, the invention provides a pharmaceutical formulation for use in administering acadesine, or the compound of formula Ia, Ia, IIIa or IVa, or a prodrug, analog, or salt thereof to a patient in need thereof, wherein the formulation is adapted for application as a spray or an aerosol.

Preparation of Preferred Novel AICA Riboside Analogs

The novel substituted imidazole analogs of the present invention can be synthesized by well known chemical reactions as demonstrated in the examples which follow. In general, compounds of formula (I) can be prepared from 4-methyl-5-nitro-1H-imidazole by the route described by Baker et al (Baker D., J. Org. Chem. 47: 3457 (1982)) to prepare 1-benzyl-5-nitro-1H-imidazole-4-carboxylic acid, followed by the additional step of reducing the nitro group to give the desired amino group at R₁. Alternatively, the elegant synthesis of AICA riboside reported by Ferris et al. (Ferris, J. P., J. Org. Chem. 50: 747 (1985), allows a versatile route to 4-substituted 5-aminoimidazoles starting with the appropriately protected riboside and diaminomaleonitrile. This route also allows for the introduction of the desired R₃ alkyl, hydrocarbyl and aryl groups by selection of the appropriate ortho ester in the cyclization reaction of the maleonitrile to the imidazole. Other desired R₃ substituents can be introduced by the methods described by Miyoshi et al. (Miyoshi T., Chem. Pharm. Bull. 24(9): 2089 (1976) for the preparation of 2-bromo and 5-amino-2-thio-1-(2,3-O-isopropylidene-β-D-ribofuranosyl)-4-imidazole carboxamide or the method of Ivanovics et al. (Ivanovics, G. A. et al., J. Org. Chem. 25: 3631 (1974)) for the preparation of 2-alkoxy, 3-amino, and 2-hydroxy (as the tautomeric 2-imidazolones) substituted 5-amino imidazole-4-carboxamides. Compounds where the desired R₁ substituent is acylamino can be prepared by acylation of the corresponding appropriately protected R₁ amino compound with the desired acyl anhydride followed by de-O-acylation with ammonia or sodium methoxide. Compounds where R₁ is alkylamino or arylamino can be prepared by reductive alkylation of the corresponding appropriately protected R₁ amino compound with the desired hydrocarbyl amine as described by Sato et al. (Chem. Pharm. Bull. 37: 1604 (1989)).

Preparation of compounds where R₆ is acyloxy or hydrocarbyloxycarboxy can be prepared selectively by reaction of the appropriate hydrocarbyl acid anhydride or hydrocarbyl chloro carbonate with the 2′,3′-O-isopropylidene protected riboside followed by removal of the isopropylidene group with dilute aqueous acid as described by Miyoshi et al. (vide supra). Compounds where R₆ is hydrocarbyloxy can be prepared from the protected 5-substituted pentoses (Snyder J. R., Carbonhydr. Res. 163: 169 (1987)), using the method of Ferris et al. (vide supra). Compounds according to formula (I) where R₆ is sulfhydryl, hydrocarbylthio or hydrocarbylamino can be prepared from the 5′-deoxy-5′-iodo-2,3′-isopropylidene imidazole riboside (Srivastava P. C., J. Med. Chem. 18: 1237 (1975)) by nucleophilic displacement of the halogen with the desired amine or mercaptan. Compounds according to formula (I) where R₆ is alkylamido or arylamido can be prepared from the corresponding 5-amino-5′-deoxyimidazole riboside by acylation with the desired alkyl or aryl acid anhydride followed by de-O-acylation with ammonia or sodium methoxide. Compounds according to formula (I) where R₆ is hydrocarbyl can be prepared from the 1-(2,3-O-isopropylidene-β-D-ribo-pento-1,5-dialdo-1,4-furanosyl)imida zoles by the Wittig reaction modification of nucleosides described by Montgomery et al. (J. Het. Chem. 11: 211 (1974)). Compounds according to formula (I) where R₆ is phosphate or a phosphate ester can be prepared by the general method of Khwaja et al. (Tetrahedron 27: 6189 (1971)) for nucleoside phosphates.

Utility

The AICA riboside analog compounds of this invention will be particularly useful in the reduction of injury during or prevention of ischemia-related events i.e. conditions that arise because of restriction of blood supply. This includes heart attack, or myocardial infarction, a situation that follows from obstruction of one or more of the coronary arteries supplying blood to the heart muscle, or myocardium, and which, if prolonged, leads to irreversible tissue damage. Compounds which, like AICA riboside, lead to increased local levels of adenosine, and thereby increasing blood flow to the ischemia myocardium, will ameliorate this tissue damage.

One current treatment for a heart attack is thrombolytic therapy, which involves administering a clot dissolving agent such as streptokinase or tissue plasminogen activator factor (tPA). However, these drugs must be used within a few hours (1-3) of the heart attack and their effectiveness decreases dramatically with longer delay. The compounds of the present invention, which can be administered prophylactically (i.e, before the event) to achieve a benefit, would therefore clearly be useful.

Angina pectoris is a condition in which the blood supply is sufficient to meet the normal needs of the heart but insufficient when the needs of the heart increase (e.g. during exercise), and/or when the blood supply becomes more limited (e.g. during coronary artery spasm). Patients with angina pectoris or with related conditions such as transient ischemic episodes or silent ischemia could similarly benefit from such an adenosinergic intervention.

In advanced coronary artery disease or persistent chest pain at rest, a number of clinical procedures are currently used to improve blood supply to the heart. These include percutaneous transluminal coronary angioplasty (PTCA), also known as angioplasty; percutaneous transluminal directional coronary atherectomy, laser atherectomy, intravascular stents and coronary artery bypass graft surgery. The compounds of this invention will also be useful as adjunctive therapies to these techniques.

Another factor lending to cardiovascular problems is abnormal heart rhythm, or arrhythmias, which lead to deficiencies in the ability of the heart to supply blood. The ability of these compounds, like AICA riboside, to reduce arrhythmias will also make them useful in suppressing this condition.

Stroke and central nervous system (CNS) trauma conditions resulting from reduced blood supply to the CNS and is thus amenable to an intervention that provides increased levels of adenosine to the compromised tissue to facilitate tissue survival. Other indications ameliorated by agents effecting regional blood flow include organ transplantation, skin flap grafting in reconstructive surgery, peripheral vascular disease, endotoxemia, hemorrhagic shock, pulmonary edema, pulmonary injury secondary to burns (thermal injury) or septicemia, pulmonary hypertension, microembolization, impotence, glomerulonephritis or progressive glomerulosclerosis, artherosclerosis, myocarditis, vasculitis and cardiomyopathies and cardiopulmonary arrest.

It is now clear that a significant component of the neurodegeneration resulting from stroke or CNS trauma is caused by increased excitatory amino acid release, which results in neurons being stimulated to death. Adenosine has been reported to inhibit excitatory amino acid release (Burke and Nadler J. Neurochem. 51: 1541 (1988)). The compounds of this invention which increase adenosine levels, therefore would also be useful in conditions where excitatory amino acids are implicated such as Huntington's chorea or Alzheimer's disease (Marangos et al. Trends Neurosci. 10: 65 (1987)) and Parkinson's disease (Sonsella et al. Science 243: 398 (1989)). These studies, together with results from experimental models of memory (Harris et al. Brain Res. 323: 132 (1984)) suggest additional utility of these compounds in treatment of disorders related to the effects of the aging process on CNS function.

Adenosine has been reported to be an endogenous modulator of inflammation by virtue of its effects on stimulated granulocyte function (Cronstein et al., J. Clin. Invest. 78: 760-770 (1986)) and on macrophage, lymphocyte and platelet function. The compounds of this invention will therefore be useful in conditions in which inflammatory processes are prevalent such as arthritis, osteoarthritis, autoimmune disease, adult respiratory distress syndrome (ARDS), inflammatory bowel disease, necrotizing enterocolitis, chronic obstructive pulmonary disease (COPD) and other inflammatory disorders.

Adenosine has been proposed to serve as a natural anticonvulsant (Lee et al., Brain Res. 321: 1650-1654 (1984); Dunwiddie, Int. Rev. Neurobiol. 27: 63-139 (1985)). Agents that enhance adenosine levels will therefore be useful for the treatment of seizure disorders. In a recent study, Marangos et al., Epilepsia 31: 239-246 (1990) reported that AICA riboside was an inhibitor of seizures in an experimental animal model.

AICA riboside analogs will also be useful in the treatment of patients who might have chronic low adenosine levels or who might benefit from enhanced adenosine, such as those suffering from autism, cerebral palsy, insomnia, anxiety, or other neuropsychiatric symptoms or those suffering from irritable bowel syndrome. Indeed, a number of studies (Komhuber and Fischer Neurosci. Lett. 34: 32 (1982); Kim et al. Eur. Neurol. 22: 367 (1983)) have linked excitatory amino acids with the pathophysiology of schizophrenia.

The compounds of this invention may also be useful in treating other conditions in which AICA riboside itself has beneficial effects. For instance, since AICA riboside has been reported to have anti-allergic actions in a guinea pig model of bronchospasm induced by antigen sensitization (Bergren et al., submitted to J. of Allergy and Clinical Immunology (1990)), AICA riboside analogs may have therapeutic benefit in the treatment of asthma, hayfever or allergic diseases.

The AICA riboside analogs of the present invention are therefore useful in the treatment of a variety of clinical situations where increasing extracellular adenosine levels and in some cases, at the same time, providing free radical scavenging and/or antioxidant activity are beneficial.

Compounds of the invention are administered to the affected tissue at the rate of from 0.01 to 3.0 μmole/min/kg, preferably from 0.1 to 1.0 μmol/min/kg. Under circumstances where longer infusions are desirable, the compounds may be administered at lower rates, e.g. 0.003 to 0.3 μmole/kg/min, preferably 0.01 to 0.1 μmole/kg/min. Use of time-release preparations to control the rate of release of the active ingredient may be preferred. These compounds are administered in a dose of about 0.01 mg/kg/day to about 200 mg/kg/day, preferably from about 0.5 mg/kg/day to about 100 mg/kg/day. Exemplary preferred doses for oral administration are 0.3 to 30 mg/kg/day, most preferably 1 to 10 mg/kg/day.

In some embodiments of the invention, a unit dose for a given compound of the invention can be administered at a dose of about 0.01 to 500 mg/kg/day, or 0.01 to 0.5, 0.25 to 1.0, 0.5 to 2.0, 1.0 to 5.0, 2.5 to 10, 5.0 to 25, 10 to 50, 25 to 75, 50 to 100, 75 to 150, 100 to 200, 150 to 300, 200 to 400, 250 to 450, or 300 to 500 mg/kg/day.

For the purposes of this invention, the compounds of the invention may be administered by a variety of transdermal means.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropylmethylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadeaethyleneoxycetanol), a condensation product of ethylene oxide with a partial ester derived from a fatty acid and a hexitol anhydride (e.g., polyoxyethylene sorbitan mono-oleate). The aqueous suspension may also contain one or more preservative such as ethyl of n-propyl p-hydroxybenzoate, one or more coloring agent, one or more flavoring agent and one or more sweetening agent, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate.

The amount of active ingredient that may be combined with the carrier material to produce a single dosage form will vary depending upon the host treated and the particular mode of administration. For example, a time-release formulation intended for oral administration to humans may contain 20 to 200 μmoles of active material compounded with an appropriate and convenient amount of carrier material which may vary from about 5 to about 95% of the total compositions. It is preferred that pharmaceutical composition be prepared which provides easily measurable amounts for administration. For example, an aqueous solution intended for intravenous infusion should contain from about 20 to about 50 μmoles of the active ingredient per milliliter of solution in order that infusion of a suitable volume at a rate of about 30 ml/hr can occur.

It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### DEFINITIONS

“Administered” or “administration” refers to the introduction of the blood clotting inhibitor to the patient. Administration refers to the giving of a dose by a person, including, for example, a health care provider or the patient himself.

“Blood clotting inhibitor” refers to any drug, agent or pharmaceutical composition that can block, prevent or inhibit the formation of blood clots or dissolves or breaks down a blood clot. A blood clotting inhibitor can be any blood clotting inhibitor currently known to those of skill in the art or one later developed. The blood clotting inhibitor can be from any drug class of blood clotting inhibitors known to those of skill in the art including, but not limited to, antiplatelet agents, thrombolytic enzymes, aggregation inhibitors, glycoprotein IIb/IIIa inhibitors, glycosaminoglycans, thrombin inhibitors, anticoagulants, heparin, low molecular weight heparins, coumarins, indandione derivatives, tissue plasminogen activators and combinations thereof. The blood clotting inhibitors can be in any pharmaceutical dosage form and administered by any route known to those of skill in the art.

“Perioperative” refers to the time period before surgery (pre-operative), after surgery (post-operative), during surgery (intra-operative), and/or any combination as described herein. For example, the blood clotting inhibitor can be administered 48 hours perioperatively; that is, the blood clotting inhibitor can be administered 48 hours before surgery (pre-operatively), 48 hours after surgery (post-operative), during surgery (intra-operative) or any combination of these administration times. The administration during the perioperative period can be a single dose or multiple doses within the perioperative time period. It will be appreciated by those of skill in the art that ‘pre-operative’ refers to the time period before surgery, ‘post-operative’ refers to the time period after surgery and ‘intra-operative’ refers to the time period during surgery.

“Long-term” refers to the time period after hospital discharge, and extending for 6 months or longer. For example, the blood clotting inhibitor can be administered at the time of discharge as one dose, and then may be continued for 6 months, one year or longer, after the perioperative period.

“Surgery” or “surgical” refers to any manual or operative methods or manipulations for the treatment or prevention of disease, injury or deformity. Surgery includes methods or manipulations conducted while a patient is under anesthesia, including local or general anesthesia. Surgery can be performed by a doctor, surgeon or dentist, generally in a hospital or other health care facility. Patients undergoing surgery can be hospitalized or ambulatory, e.g., out-patient surgery. Surgery does not include percutaneous intervention (PTI) or percutaneous transluminal coronary angioplasty (PTCA).

“Coronary artery bypass graft” or “CABG” refers to cardiac surgery wherein one or more bypass grafts are implanted between the aorta and the coronary blood vessel, commonly using saphenous veins or internal mammary arteries as grafts. “Vein graft CABG” refers to CABG surgery wherein a saphenous vein(s) is used for grafting. “Artery graft CABG” refers to CABG surgery wherein an internal mammary artery (arteries) is used for grafting.

Timing of Administration

The blood clotting inhibitor can be administered perioperatively; that is, before surgery, after surgery and/or during surgery, or any combination as described herein. For example, if the half-life of the drug is long (24-48 hours), the blood clotting inhibitor can be administered as one dose within 48 (or 24) hours prior to surgery with repeated doses during or after surgery. Drugs with shorter half-lives can be given sooner before surgery and then be administered during or after surgery. In some patients, and some circumstances, the treating physician may decide to suspend preoperative treatment, and only start administration postoperatively, e.g., 48 hours after surgery, after wound closure to assure that no bleeding has occurred in the field (no open blood vessels) before starting anti-clotting therapy. Such immediate postoperative administration of a blood clotting inhibitor is within the scope of the invention.

Perioperative administration includes the time period before surgery (pre-operative), after surgery (post-operative), during surgery (intra-operative), and/or any combination as described herein. For example, the blood clotting inhibitor can be administered 6 months, 3 months, 1 month, week, 96 hours, 48 hours or less perioperatively; that is, the blood clotting inhibitor can be administered 6 months, 3 months, 1 month, 1 week, 96 hours, 48 hours or less before surgery, 6 months, 3 months, 1 month, 1 week, 96 hours, 48 hours or less after surgery, or both 6 months, 3 months, 1 month, 1 week, 96 hours, 48 hours or less before and after surgery. In addition, the blood clotting inhibitor can be administered, for example, 36, 24, 12, 8, 6, 4, 2 or 1 hour perioperatively; that is the blood clotting inhibitor can be administered, for example, 36, 24, 12, 8, 6, 4, 2 or 1 hour before surgery and/or 36, 24, 12, 8, 6, 4, 2 or 1 hour after surgery and/or during surgery. One can administer the blood clotting inhibitor for an equal number of hours pre and post surgery. For example, one can administer the blood clotting inhibitor 48 hours prior to surgery and 48 hours after surgery. One can administer the blood clotting inhibitor for an unequal number of hours pre and post surgery. For example, one can administer the blood clotting inhibitor 48 hours prior to surgery and 24 hours after surgery. One can administer the blood clotting inhibitor, for example, 36 hours prior to surgery and 36 hours after surgery. One can administer the blood clotting inhibitor 36 hours prior to surgery and 12 hours after surgery. One can administer the blood clotting inhibitor, for example, 12 hours prior to surgery and 12 hours after surgery. One can administer the blood clotting inhibitor, for example, 8 hours prior to surgery and 8 hours after surgery. One can administer the blood clotting inhibitor, for example, 6 hours prior to surgery and 8 hours after surgery. One can administer the blood clotting inhibitor, for example, 6 hours prior to surgery and 6 hours after surgery. One can administer the blood clotting inhibitor, for example, 8 hours prior to surgery and 4 hours after surgery. One can administer the blood clotting inhibitor 4 hours prior to surgery and 4 hours after surgery. One can administer the blood clotting inhibitor 2 hours prior to surgery and 8 hours after surgery. One can administer the blood clotting inhibitor 4 hours prior to surgery and 1 hour after surgery. One can administer the blood clotting inhibitor, for example, 24 hours prior to surgery and during surgery. One can administer the blood clotting inhibitor, for example, during surgery and 6 hours after surgery.

Administration in the perioperative period can be a single, one time dose or multiple doses of the blood clotting inhibitor. In certain embodiments, perioperative administration can be continuous, uninterrupted administration of the blood clotting inhibitor (e.g. a continuous infusion or transdermal delivery). In another embodiment, perioperative administration is single or multiple discreet administration(s) within the perioperative time frame (e.g. a single dose given within the perioperative period or multiple doses given within the perioperative period). In one embodiment, the blood clotting inhibitor can be administered within 6 days, 5 days, 4 days, 3 days, 2 days or 1 day perioperatively. In another embodiment, the blood clotting inhibitor can be administered within 48 hours, 36 hours, 24 hours, 12 hours, 8 hours, 6 hours or 1 hour perioperatively.

The blood clotting inhibitor can be administered during surgery, for example, contemporaneously with the use or discontinuation of cardiopulmonary bypass or contemporaneously with reperfusion of an ischemic area. Administration can be continued long term for example, after surgery, following discharge from hospital and for six months, one year or longer post-operatively.

In certain embodiments, when the patient is on chronic blood clotting inhibitor therapy prior to surgery, the blood clotting inhibitor is not discontinued pre-operatively, in contrast to standard practice.

Perioperatively, the patient need not be conscious for administration of the blood clotting inhibitor. For example, the blood clotting inhibitor can be given during surgery while the patient is under anesthesia. During some ambulatory or outpatient surgeries, the patient remains conscious and in such a situation, the blood clotting inhibitor can be given during surgery when the patient is conscious.

Such therapy can be continued after discharge. In the course of long-term treatment, as described above, the formulation and dosage can be continued or adjusted, or the type of blood clotting inhibitor can be changed to another blood clotting inhibitor.

Surgery and Surgical Complications

The present invention provides methods of preventing or reducing post-surgical morbidity and mortality. In certain aspects the methods comprise the perioperative administration of a blood clotting inhibitor to prevent or reduce post-surgical complications. The blood clotting inhibitor can be administered perioperatively; that is prior to, during and/or after surgery, and after hospital discharge. Significantly, the prevention or reduction of post-surgical morbidity and mortality extends beyond hospitalization.

Surgery refers to any manual or operative methods or manipulations for the treatment or prevention of disease, injury or deformity. Surgery includes methods conducted while a patient is under anesthesia, including local or general anesthesia. Surgery can be performed by a doctor, surgeon or dentist, generally in a hospital or other health care facility. Patients undergoing surgery can be hospitalized or ambulatory, e.g., out-patient surgery. For purposes of this invention surgery includes, but is not limited to abdominal surgery (e.g. surgery of the abdominal viscera), bench surgery, (e.g. surgery performed on an organ that has been removed from the body, after which it can be reimplanted), cardiac (e.g. surgery of the heart), cerebral (e.g. surgery upon the brain), cineplastic (e.g. surgery to create a tunnel through a muscle adjacent to the stump of an amputated limb, to permit use of the muscle in operating a prosthesis), cosmetic (e.g. surgery to improve a patient's appearance by plastic restoration, correction or removal of blemishes), dentofacial (e.g. surgery involving defects of the face and structures of the mouth), neurological (e.g. surgery involving the peripheral or central nervous system), oral (e.g. surgery involving defects of the mouth, jaws and associated structures), orthopedic (e.g. surgery dealing with bones and bony structures), pelvic (e.g. surgery involving the pelvis, predominately obstetrical and gynecological), plastic (e.g. surgery involving the restoration, reconstruction, correction or improvement in the shape and appearance of body structures that are defective, damaged or misshapened by injury, disease, or growth and development) or rectal (e.g. surgery of the rectum), urological (e.g. surgery related to the genitourinary system, predominately in males), vascular (e.g. surgery of the blood vessels), and surgery related to otolaryngology (e.g. surgery of the ears, nose, throat or related structures). The surgery can be conservative (e.g. surgery to preserve or remove with minimal risk, diseased or injured organs, tissues, or extremities) or radical (e.g. surgery designed to extirpate all areas of locally extensive disease and adjacent zones of lymphatic drainage). In certain embodiments, the surgery can be cardiac surgery, including cardiac valve replacement, heart and heart-lung transplant, and implantation of artificial heart devices and defibrillators, valve replacement or valve repair and congenital surgery.

In certain embodiments, when the cardiac surgery is CABG, the surgery can be coronary artery bypass grafting using saphenous veins or internal mammary arteries, referred to herein as vein graft CABG or artery graft CABG, respectively. In one embodiment, when the surgery is vein graft CABG, the blood clotting inhibitor is not aspirin administered from the time beginning 12 hours pre-operatively through seven hours post-operatively. In another embodiment, when the surgery is vein graft CAB G, the blood clotting inhibitor is not dipyridamole administered from the time beginning 48 hours pre-operatively through 24 hours post-operatively. See, Goldman, et al., 1988, Circulation 77:1324-32; Chesebro, et al., 1982, NEJM 307:73-8; Chesebro, et al., 1984, NEJM 310:209-14. In another embodiment, when the surgery is vein graft CABG, the blood clotting inhibitor is not ticlopidine or aprotinin. See, Drug Facts and Comparisons, updated monthly, September, 2002, Facts and Comparisons, Wolters Kluwer Company, St. Louis, Mo.

In certain embodiments, when the cardiac surgery is artery graft CABG, the blood clotting inhibitor is not aprotinin.

The invention can be used on a wide variety of surgeries, including, but not limited to, cardiac, abdominal, neurological, gynecological, orthopedic, urological, vascular, and surgery related to otolaryngology. More specifically, surgery includes, small and large bowel resection, appendectomy, laparoscopy, paracentesis, transurethral resection of the prostate (TURP), hysterectomy, tuba ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, liposuction and the like.

Ambulatory or outpatient surgery includes surgery for which hospitalization and/or general anesthesia is generally not required. Such surgeries include placement of myringotomy tubes, hemorrhoidectomy and the like.

The invention can reduce post-surgical morbidity and mortality during the post-surgical hospitalization recovery period and after discharge from hospital. The post-surgical morbidity and mortality can be from any surgical complication. Complications of surgery can be cardiac (myocardial infarction, congestive heart failure, serious cardiac dysrhythmias, ischemia) neurological (stroke, encephalopathy, cognitive dysfunction, transient ischemic attacks, seizures), renal (failure, dysfunction or renal death), gastrointestinal (infarction, ileus, ischemia, mesenteric thrombosis or GI death), pulmonary (failure, respiratory distress syndrome, edema), and the like.

Blood Clotting Inhibitor

The present invention provides methods of preventing or reducing post-surgical morbidity and mortality. In certain aspects the methods comprise the perioperative administration of a blood clotting inhibitor to prevent or reduce post-surgical complications. The blood clotting inhibitor can be administered perioperatively; that is prior to, during and/or after surgery, and after hospital discharge.

The blood clotting inhibitor of the present invention can be any drug, agent or pharmaceutical composition that prevents or inhibits blood clotting. The inhibitor can act by preventing or inhibiting blood clot formation by any of a variety of mechanisms including reduction of blood clotting factors or reducing platelet activation or aggregation, or mitigating the effects of instigating factors, such as inflammation or stress. The blood clotting inhibitor can also act by breaking down or dissolving a blood clot after formation. It will be apparent to those of skill in the art that there are several classes of blood clotting inhibitor, including antiplatelet agents, thrombolytic enzymes, aggregation inhibitors, glycoprotein IIb/IIIa inhibitors, glycosaminoglycans, thrombin inhibitors, anticoagulants, heparins, low molecular weight heparins, coumarins, indandione derivatives and tissue plasminogen activators. See, The Physicians' Desk Reference (56^{th} ed., 2002) Medical Economics; Mosby's Drug Consult, 2002, Elsevier Science; Goodman and Gilman's The Pharmacologic Basis of Therapeutics, (9^{th} ed. 1996) Pergamon Press; Drug Facts and Comparisons, updated monthly, September, 2002, Facts and Comparisons, Wolters Kluwer Company, St. Louis, Mo.

For the purposes of this invention, any drug, agent or pharmaceutical composition that prevents or inhibits the formation of blood clots or dissolves or breaks down a blood clot is suitable for use in the present invention. Such a blood clotting inhibitor can be, for example, cilostazol (PLETAL®, Otsuka), clopidogrel (PLAVIX®, Sanofi), ticlopidine (TICLID®, Syntex), tirofiban (AGGRASTAT®, Merck), eptifibatide (INTEGRILIN®, COR Therapeutics), abciximab (REOPRO®, Eli Lilly), anagrelide (AGRYLIN®, Roberts), dipyridamole (PERSANTIN®, Boehringer Ingelheim), aspirin (ECOTR®, and others), dipyridamole/aspirin (AGGRENOX®, Boehringer Ingelheim), dalteparin (FRAGMIN®, Pharmacia), enoxaparin (LOVENOX®, Aventis), tinzaparin (INNOHE®, DuPont), heparin (various), danaparoid (ORGANON®, Organon), antithrombin III (THROMBATE®, Bayer), lepirudin (REFLUDAN®, Hoechst-Marion Roussel), argatroban (ACOVA®, SmithKlineBeecham), bivalirudin (ANGIOMAX®, Medicines Company), warfarin (COUMADIN®, DuPont) anisidione (MIRADON®, Schering), alteplase (ACTIVASE®, Genetech), reteplase (RETAVASE®, Boehringer Mannheim), tenecteplase (TNKASE®, Genentech), drotrecogin (XIGRIS®, Eli Lilly), anistreplase (EMINASE®, Roberts), streptokinase (STREPTASE®, Astra), urokinase (ABBOKINASE®, Abbott) and combinations thereof.

It will be appreciated by those of skill in the art that blood clotting inhibitors are used for the treatment of occluded catheters and for the maintenance of patency of vascular access devices. Heparin, urokinase, streptokinase and alteplace are generally employed for such uses. The use of blood clotting inhibitors for the treatment of occluded catheters and for the maintenance of patency of vascular access devices is not within the scope of the invention.

In certain embodiments where the blood clotting inhibitor is a low molecular weight heparin, the surgery is preferably not hip replacement, knee replacement or abdominal surgery. When the drug is dalteparin, the dose is preferably not 2500 IU subcutaneously once daily, starting 1 to 2 hours preoperatively and repeating once daily for 5-10 post-operatively or 5000 IU subcutaneously the evening before surgery and repeated once daily for 5-10 days postoperatively. When the drug is enoxaparin, the dose is preferably not 40 mg once daily subcutaneously given initially 9 to 15 hours prior to surgery and continued for 21 days or 40 mg once daily subcutaneously starting 2 hours prior to surgery and continued for 7 to 10 days; 12 days if tolerated.

In certain embodiments where the blood clotting inhibitor is heparin, the surgery is preferably not abdominothoracic or cardiac surgery. When the drug is heparin, the dose is preferably not 5000 Units subcutaneously 2 hours before surgery and 5000 Units every 8 to 12 hours thereafter for 7 days or until the patient is fully ambulatory. When the drug is heparin, the dose is preferably not 150 Units/kg for patients undergoing total body perfusion for open heart surgery. When the drug is heparin, the dose is preferably not 300 Units/kg for procedures less than 60 minutes or 400 Units/kg for procedures longer than 60 minutes.

In certain embodiments where the blood clotting inhibitor is danaparoid, the surgery is not elective hip replacement surgery. When the drug is danaparoid, the dose is preferably not 750 anit-Xa units twice daily subcutaneously beginning 1 to 4 hours preoperatively and then not sooner than 2 hours after surgery continued for 7-10 days postoperatively.

In certain embodiments where the blood clotting inhibitor is warfarin, the surgery is preferably not cardiac valve replacement surgery. When the drug is warfarin, the dose is preferably not 1 mg daily, up to 20 days preoperatively.

In certain embodiments, when the cardiac surgery is vein graft CABG, the blood clotting inhibitor is not aspirin administered within 12 hours pre-operatively through seven hours post-operatively. In certain embodiments, when the cardiac surgery is vein graft CABG, the blood clotting inhibitor is not dipyridamole administered within 48 hours pre-operatively through 24 hours post-operatively. See, Goldman, et al., 1988, Circulation 77:1324-32; Chesebro, et al., 1982, NEJM 307:73-8; Chesebro, et al., 1984, NEJM 310:209-14. In certain other embodiments, when the cardiac surgery is vein graft CABG, the blood clotting inhibitor is not ticlopidine or aprotinin. See, Drug Facts and Comparisons, updated monthly, September, 2002, Facts and Comparisons, Wolters Kluwer Company, St. Louis, Mo.

Aprotinin is indicated for CABC surgery in one of two dosing regimens, regimen A or regimen B. Regimen A is administration of a 2 million KIU (kallikrein inhibitor units) intravenous loading dose; 2 million KIU into the cardiopulmonary bypass machine (known as pump prime volume) and 500,000 KIU/hr of operation time as a continuous maintenance intravenous infusion. Regimen B is administration of a 1 million KIU intravenous loading dose, 1 million KIU into the pump prime volume and 250,000 KIU/hr of operation time as a continuous maintenance intravenous infusion. Administration of aprotinin begins after anesthetic induction but prior to stemotomy and is continued until surgery is complete and the patient leaves the operating room. Drug Facts and Comparisons, updated monthly, September, 2002, Facts and Comparisons, Wolters Kluwer Company, St. Louis, Mo. In certain embodiments when the surgery is vein graft or artery graft CABG, the blood clotting inhibitor is not aprotinin.

The blood clotting inhibitor can be a combination of two or more blood clotting inhibitors. Combinations of blood clotting inhibitors can include blood clotting inhibitors from more than one drug class as described herein. In addition, the combination of blood clotting inhibitors can include different routes of administration for each blood clotting inhibitor. The combination of blood clotting inhibitors can be administered simultaneously or contemporaneously. In addition, the combination of blood clotting inhibitors can be administered separately.

Dosage, Formulation and Administration

The blood clotting inhibitor described herein, can be administered into a patient for the reduction of mortality and morbidity following surgery to produce contact of the blood clotting inhibitor with the blood clotting inhibitor's site of action in the body of the patient. It will be apparent to those of skill in the art that a pharmaceutical composition can be generally administered with a pharmaceutical carrier.

The dose administered will, of course, vary depending upon known factors, such as: the pharmacodynamic characteristics of the particular blood clotting inhibitor; the age, health, height and weight of the patient; the kind of concurrent treatment(s); the frequency of treatment(s); and the effect desired. The dose of the blood clotting inhibitor need not remain constant but can be adjusted according to parameters that are well known to those of skill in the art. In addition, the dose of blood clotting inhibitor can be sub- or supra-therapeutic.

Transdermal delivery systems manufactured as an adhesive disc or patch that slowly releases the active ingredient for percutaneous absorption may be used. To this end, permeation enhancers may be used to facilitate transdermal penetration of the blood clotting inhibitor. For example, for transdermal administration, the compounds herein may be combined with skin penetration enhancers, such as propylene glycol, polyethylene glycol, isopropanol, ethanol, oleic acid, N-methylpyrrolidone, dimethyl sulfoxide, and the like, which increase the permeability of the skin to the compounds, and permit themto penetrate through the skin and into the bloodstream. The compounds herein may also be combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The compounds may be administered transdermally e.g., at a site of a wound in a subject. Alternatively, the compounds may be administered transdermally at a site other than the affected area, in order to achieve systemic administration.

The use of a device for providing continuous and controlled topical delivery of active agents through skin or mucosa is well known. These devices, often referred to as transdermal drug delivery systems, reduce or obviate effects associated with other routes of drug administration—oral administration in which the drug may not be tolerated well by the digestive tract and is often required to be multiple daily dosed in order to achieve therapeutic levels (e.g., low bioavailability due to degradation or metabolism); administration by ointment, gel or cream in which the drug may be inadequately dosed or overdosed causing wide fluctuations in blood levels or therapeutic effect at the site of application, or may cause undesirable side effects of the absorbing tissues as well as interfering with the clothing and activities of the user; and administration by injection in which the delivery is typically unpleasant or painful and requiring assistance of a medical professional. Trandsermal patches that can be utilized in the present invention include those disclosed in numerous technical publications such as the following: U.S. Pat. Nos. 3,598,122; 3,598,123; 3,731,683; 3,797,494; 4,031,894; 4,201,211; 4,286,592; 4,314,557; 4,379,454; 4,435,180; 4,559,222; 4,568,343; 4,573,995; 4,588,580; 4,645,502; 4,704,282; 4,788,062; 4,816,258; 4,849,226; 4,908,027; 4,943,435; 5,004,610; 5,252,335; 5,344,656; 5,904,953; 5,662,624; 6,585,670; 6,890,553; 5,662,925; 6,096,339; 6,168,801; 5,645,854; and 5,229,129; 5,344,656; 7,063,859; 7,049,479; and 7,063,859 the disclosures of which are incorporated herein by reference in their entirety.

Generally speaking, transdermal drug delivery systems are commonly either reservoir-type or matrix-type devices. Both types of devices include a backing layer that forms the outer surface of the finished transdermal device and which is exposed to the environment during use, and a release liner or protective layer that forms the inner surface and which covers the adhesive means for affixing the devices to the skin or mucosa of a user. The release liner or protective layer is removed prior to application, exposing the adhesive means which is typically a pressure-sensitive adhesive. The active agent is located between the release liner and backing layer, usually solubilized or dispersed in a solvent or carrier composition. In some embodiments, the outer surface of the transdermal device (e.g., patch) is adapted to associate with a second component, such as a heating compartment (e.g., electrical or chemical means for providing controlled and consistent increase in temperature). Examples of heating components for transdermal systems that can be utilized with compositions and methods of the invention include those disclosed in U.S. Pat. Nos. 6,893,453, 5,718,955, 5,279,544, 5,232,702, 5,904,935, 5,662,624, 6,170,095, 6,485,506, 6,921,374, 6,955,819 and 7,094,228.

In a reservoir-type device, the active agent is isolated from the adhesive means used to affix the device to the user. Traditionally, a reservoir system referred to a device having a pocket or “reservoir” serving to hold the active agent and was formed in or by the backing layer itself. Such a backing layer is impermeable, occlusive and typically rigid. A peripheral adhesive layer is then used to affix the device to the user. While such devices are still in use today, the term reservoir has become known as a device which employs one or more permeable layers, such as rate controlling membranes, and drug permeable adhesives layers, laminated over the reservoir (which is typically nothing more than another layer containing the drug in a carrier composition), in an effort to more effectively control the delivery rate of the active agent and attachment of the device to the user.

A matrix-type device generally comprises the active agent solubilized or dispersed in a carrier formulation which functions as both the drug carrier and the adhesive means of applying the system to the skin or mucosa. Such devices are described, for example, in U.S. Pat. Nos. 4,994,267; 5,474,783 and 5,656,286.

In order to produce a transdermal drug delivery system intended to deliver an active agent locally, for example, to treat a disorder, flexibility and comfort become critical to the system's characteristics since many of the application sites are greatly contoured or subject to frequent movement. The backing layer should generally be sufficient thickness to provide sufficient strength and support for the active agent carrier composition, and yet also retain elongation properties to provide sufficient flexibility to maintain contact with the application site, and optionally to maintain comfort when worn over extended periods of time. In some embodiments, a “soft” backing layer, similar in appearance to the padded covering used in common stick-on bandages, such as cloth, woven and foamed materials, are used to achieve sufficient elongation and support properties.

Variations exist in transdermal systems incorporating these soft backings. In addition to serving as an outer protective surface, these system backing layers can also serve as the depot or storage location for receiving and retaining some or all of the active agent. In one embodiment, the backing layer is or becomes infiltrated with the drug itself in alternative embodiments, or with a solution or mixture of the drug and a suitable solvent or polymer carrier. Typically, the active agent carrier is in a non-finite form such as a gel, ointment, liquid and the like. In some embodiments, because the backing layer can receive and absorb the active agent, (a) stability and potency of the drug and (b) porosity or occlusiveness of the backing layer which affects the degree of penetration of the drug and/or carrier composition into the backing layer, can be modified to effect different delivery rates for different active agents.

For example, U.S. Pat. No. 5,741,510 describes a transdermal patch comprising a porous backing layer for receiving a pressure-sensitive drug containing hydrogel. The drug containing hydrogel is required to substantially penetrate into the backing layer. In order to prevent the hydrogel from becoming too viscous to properly penetrate the backing layer, the hydrogel requires chilling to keep it sufficiently fluid. The porosity of the backing layer consequently becomes important to achieve the necessary degree of penetration as well as to provide a non-occlusive patch (i.e., permit permeation of water vapor out of the system). Since the hydrogel also functions as the adhesive means for attachment of the device to the user, its application must be further controlled so as to also remain sufficiently on the surface of the backing layer.

U.S. Pat. No. 5,635,201 discloses a method and apparatus for manufacturing a wound dressing. The method includes coating an upper surface of a perforated backing material with a curable silicone mixture, blowing cold air onto the underside of the backing material, and applying heat to the silicone mixture until it is cured to a silicone gel. The cold air is applied by an air blowing unit to remove an applied silicone mixture from pores in the backing material, thereby maintaining the porosity of the wound dressing. The cold air further prevents the silicone mixture from curing before it has time to spread over the backing material.

In order to prevent the active agent and any solvents and/or polymer carrier for the active agent from passing outwardly and through the outer surface of the backing layer, some devices add a barrier layer to such outer surface. For example, U.S. Pat. No. 5,716,621 discloses the use of a moisture vapor permeable barrier layer bonded to the outer surface of a foam backing layer. The backing layer is used as the depot for the drug and its carrier composition. Since it is essential that the barrier layer be moisture vapor permeable, the patent further teaches nonadhesive techniques as the preferred method of bonding the barrier layer to the upper surface of the backing layer. Since such a transdermal system does not use a drug carrier composition which can also function to affix the system to the user, a separate adhesive means must be deposited on the inner surface of the backing layer.

Suitable adhesives (for formulation with or without a drug compound disclosed herein) include any natural or synthetic material that is capable of adhering to a surface and remaining permanently tacky. Preferred adhesives are pressure-sensitive adhesives and include all of the non-toxic natural and synthetic polymers known or suitable for use in transdermal systems as hydrophobic adhesives, such as polyacrylates, polysiloxanes, silicones, rubbers, polyisobutylenes, polyvinylethers, polyurethanes, styrene/butadiene polymers, polyether block amide copolymers, ethylene/vinyl acetate copolymers, vinyl acetate based adhesives, and combinations and mixtures thereof.

An adhesive is a pressure-sensitive adhesive within the meaning of the term as used herein if it has the properties of a pressure-sensitive adhesive per se or if it functions as a pressure-sensitive adhesive by admixture with tackifiers, plasticizers or other additives.

For example, preferred pressure-sensitive adhesives are low molecular weight adhesives. The term “low molecular weight” means that the adhesive is not substantially cross-linked, and has a molecular weight of less than about 1,000,000.

In general, a low molecular weight adhesive has a low viscosity and/or internal cohesiveness, thereby permitting the adhesive to remain fluid enough to migrate slightly into the backing layer when applied, rather than remain solely on the surface of the backing layer 14. This desired fluidity and slight penetration of the adhesive into backing layer helps to ensure a bond of sufficient strength to prevent or minimize delamination between the barrier film layer 13 and the backing layer 14. A low molecular weight adhesive tends to further remain more pliable and therefore allows for greater flexibility and comfort during topical application, which is essential to achieve optimal performance of the transdermal system.

While a high molecular weight adhesive could be heated or cooled, as the case may be, in order to prevent the adhesive from becoming too viscous to permit penetration into the backing layer 14, use of such an adhesive when cured or hardened may interfere with the transdermal system's desired ability to flex and stretch during topical application.

In one embodiment, adhesives utilized with the patches of the invention are polyacrylates of one or more monomers of acrylic acids or other copolymerizable monomers. Polyacrylate adhesives also include polymers of alkyl acrylates and/or methacrylates and/or copolymerizable secondary monomers, or monomers with functional groups, and in particular hydroxy functional groups. The term “polyacrylate” is intended to be used interchangeably with the terms acrylic, acrylate and polyacrylic as used herein and as known in the art. Such polyacrylate adhesives preferably have a molecular weight of about 250,000 to about 750,000, and more preferably of about 300,000 to about 600,000.

Suitable low molecular weight acrylic adhesives are commercially available and include those sold under the trademarks DURO-TAK® 387-2510/87-2510 by National Starch and Chemical Company, Bridgewater, N.J., and GELVA® Multipolymer Solution 1151 by Solutia, Inc., St. Louis, Mo.

The appropriate amount of adhesive to be used for joining barrier film layer 13 to backing layer 14 is that amount which will provide a bond of sufficient strength between the barrier film layer 13 and the backing layer 14 to prevent delamination during topical application of the system, and more importantly, upon removal of the system from the application site. Such amount can be determined by routine testing.

In some embodiments of the invention, the low molecular weight acrylic adhesives can be applied to a thickness from about 2.0 mils to about 10.0 mils, and more preferably from about 4.0 mils to about 7.0 mils or, on a dry weight basis, from about 2.0 mg/cm² to about 7.5 mg/cm², and more preferably from about 2.5 mg/cm² to about 5.0 mg/cm².

In other embodiments, if the adhesive is incompatible with the backing layer, the attachment of the transdermal device to the user will be less than satisfactory, especially considering such devices are often desired to be worn continuously for an extended period of time and must be maintained properly in place throughout the entire period if the device is to effectively deliver a therapeutic amount. It is preferred that the adhesive generally remain on the surface of the backing layer rather than fill or penetrate the cells or micropores of the backing layer.

It is preferred that the adhesive keep the transdermal device in intimate contact with the skin or mucosa by anchoring properly to both the backing layer and to the skin or mucosa, but without interfering with the backing layer's function of being a drug depot.

In general, an active ingredient containing layer can comprise any of the various drug delivery configurations used in transdermal/dermal/transmucosal/mucosal delivery systems. Thus, it can be an active ingredient containing adhesive layer. Alternatively, it can comprise an active ingredient containing gel layer, or a membrane mediated active ingredient containing gel layer. In a membrane mediated system, the active ingredient can be in liquid form, as for example contained in a solution, where the rate mediated membrane comprises or is part of a pouch containing the liquid. A patch backing member may include an adhesive layer to which gel layer or membrane pouch is adhered. As for compounds disclosed herein as active agents, a method for incorporating the active agent into an adhesive or gel layer is disclosed in U.S. Pat. No. 6,576,712.

The blood clotting inhibitor can be administered by any suitable route known to those of skill in the art that ensures bioavailability in the circulation. Administration can be achieved by parenteral routes of administration, including, but not limited to, intravenous (IV), intramuscular (IM), intradermal, subcutaneous (SC), and intraperitoneal (IP) injections. In certain embodiments, administration is by a bypass machine, perfuser, infiltrator or catheter. In certain embodiments, the blood clotting inhibitor is administered by injection, by a subcutaneously implantable pump or by a depot preparation, in doses that achieve a therapeutic effect. Suitable dosage forms are further described in Remington's Pharmaceutical Sciences, 1990, 17^{th} ed., Mack Publishing Company, Easton, Pa., a standard reference text in this field, which is incorporated herein by reference in its entirety.

The effective amount of clotting agent to be administered in combination with the AICA riboside and/or analogs thereof may vary depending on the type of surgery, condition of the patient, age of the patient, patient's weight, medical history of the patient, the manner of administration and the judgment of the prescribing physician. It will be appreciated by one of skill in the art that the degree of blood anticoagulation can be monitored by laboratory values such as prothrombin time (PT) and partial thromboplastin time (PTT). Determination of an effective amount is well within the capabilities of those skilled in the art, especially in light of the detailed disclosure provided herein.

The administration of a blood clotting inhibitor may be repeated intermittently. The blood clotting inhibitor can be administered alone or in combination with other drugs, for example, other presurgical drugs such as antibiotics or anesthetics.

Blood Clotting Inhibitor and Acadesine Combination

In another aspect of the invention, a method for preventing or reducing adverse effects in a patient undergoing surgery is provided, wherein acadesine, or a prodrug, analog, or salt thereof is first administered and then a blood clotting inhibitor is administered. In one embodiment, the blood clotting inhibitor is administered during the administering of the acadesine, or a prodrug, analog, or salt thereof. In one embodiment, the acadesine, or a prodrug, analog, or salt thereof is administered at a total dose of about 10 mg/kg to about 200 mg/kg. Another aspect provides a method of preventing or reducing adverse effects in a patient undergoing non-vascular surgery, wherein acadesine, or a prodrug, analog, or salt thereof is administered and then a blood clotting inhibitor is administered. The invention can be used on a wide variety of non-vascular surgeries, including, but not limited to, cardiac, abdominal, neurological, gynecological, orthopedic, urological, vascular, and surgery related to otolaryngology. More specifically, non-vascular surgery includes, small and large bowel resection, appendectomy, laparoscopy, paracentesis, transurethral resection of the prostate (TURP), hysterectomy, tuba ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, liposuction and the like.

In one embodiment, the blood clotting inhibitor is aspirin. In one embodiment, the patient undergoing surgery or non-vascular surgery has had a past myocardial infarction. In another embodiment, the past myocardial infarction occurred within the last 24, 36, or 48 months prior to the surgery.

Another aspect of the invention provides a method of preventing or reducing adverse effects in a patient undergoing CABG surgery by first administering acadesine, or a prodrug, analog, or salt thereof and then administering a blood clotting inhibitor. In one embodiment, the administering of the blood clotting inhibitor occurs during the administering of the acadesine, or a prodrug, analog, or salt thereof. In another embodiment, the acadesine, or a prodrug, analog, or salt thereof is administered at a total dose of about 10 mg/kg to about 200 mg/kg. Another embodiment provides administration of acadesine, or a prodrug, analog, or salt thereof at 0.1 mg/kg/minute. Another embodiment provides administration of acadesine, or a prodrug, analog, or salt thereof over a period of about seven hours. Another embodiment provides administration of aspirin at a dosage of about 400 mg to about 5 g. Another embodiment provides administration of aspirin at least once within 48 hours after surgery.

Another aspect of the invention provides a pharmaceutical formulation comprising acadesine, or a prodrug, analog, or salt thereof, aspirin, and a pharmaceutically acceptable carrier, diluent, or excipient, wherein the formulation provides a patient with a blood plasma concentration between about 1 μg/ml to about 20 μg/ml over a period of about seven hours and aspirin at a dose of about 40 mg to about 5 g. In another embodiment, the invention provides a method of preventing or reducing adverse effects in a patient undergoing surgery by administering the above pharmaceutical formulation within 48 hours of surgery. In another embodiment, the surgery is CABG surgery.

The present invention provides a method for intradermally delivering the therapeutic compounds described herein. The terms “intradermal”, “intracutaneous”, “intradermally” and “intracutaneously” are used herein to mean that the antigenic agent (e.g., a vaccine antigen) and adjuvant are delivered into the skin, and specifically into the epidermis layer and/or underlying dermis layer of the skin.

The term “microprojections” refers to piercing elements which are adapted to pierce or cut through the stratum corneum into the underlying epidermis layer, or epidermis and dermis layers, of the skin of a living animal, particularly a human. The piercing elements should not pierce the skin to a depth which causes bleeding. Typically the piercing elements have a microprojection length of less than 500.mu.m, and preferably less than 250.mu.m. The microprojections typically have a width of about 75 to 500.mu.m and a thickness of about 5 to 50.mu.m. The microprojections may be formed in different configurations and/or shapes, such as needles, hollow needles, blades, pins, punches, and combinations thereof.

The term “microprojection array” as used herein refers to a plurality of microprojections arranged in an array for piercing the stratum corneum. The microprojection array may be formed by etching or punching a plurality of microprojections from a thin sheet and folding or bending the microprojections out of the plane of the sheet to form a configuration such as that shown in FIG. 1 and in Trautman et al., U.S. Pat. No. 6,083,196. The microprojection array may also be formed in other known manners, such as by forming one or more strips having microprojections along an edge of each of the strip(s) as disclosed in Zuck, U.S. Pat. No. 6,050,988. Other microprojection arrays, and methods of making same, are disclosed in Godshall et al., U.S. Pat. No. 5,879,326 and Kamen, U.S. Pat. No. 5,983,136. The microprojection array may also be in the form of a plurality of hollow needles which hold a dry antigenic agent and adjuvant.

The intradermal vaccine of the present invention includes a microprojection array having a plurality of stratum corneum-piercing microprojections extending therefrom and having a reservoir containing an antigenic agent (e.g., a vaccine antigen) and an immune response augmenting adjuvant. The reservoir is positioned, relative to the microprojections in the microprojection array, so that the reservoir is in antigenic agent-transmitting and adjuvant-transmitting relation to the slits cut through the stratum corneum by the piercing microprojections. In one embodiment, the reservoir can be a material (e.g., a gel material) in the form of a thin polymeric film laminated on the skin proximal or skin distal side of the microprojection array. Reservoirs of this type are disclosed in Theeuwes et al. WO 98/28037, the disclosures of which are incorporated herein by reference. More preferably, the antigenic agent and adjuvant are in a coating applied directly on the microprojections, most preferably on the piercing tips of the microprojections. Suitable microprojection coatings and apparatus useful to apply such coatings are disclosed in U.S. patent application Ser. Nos. 10/045,842 filed Oct. 26, 2001; 10/099,604 filed Mar. 15, 2001; and another application filed concurrently herewith and claiming dependency from U.S. provisional application Ser. No. 60/285,576 filed Apr. 20, 2001, the disclosures of which are incorporated herein by reference. The microprojections are adapted to pierce through the stratum corneum into the underlying epidermis layer, or epidermis and dermis layers, but preferably do not penetrate so deep as to reach the capillary beds and cause significant bleeding. Typically, the microprojections have a length which allows skin penetration to a depth of less than about 400.mu.m, and preferably less than about 300.mu.m. Upon piercing the stratum corneum layer of the skin, the antigenic agent and adjuvant contained in the coating are released into the skin for vaccination therapy.

The microprojection array intradermal vaccine containing the therapeutic compounds of the present invention is preferably applied to the skin of the patient under impact conditions. For example a biased (e.g., spring driven) impact applicator of the type described in Trautman et al. U.S. patent application Ser. No. 09/976,798 filed Oct. 12, 2001, the disclosures of which are incorporated herein by reference, can be used to apply the coated microprojection arrays of the present invention. Most preferably, the coated microprojection array is applied with an impact of at least 0.05 joules per cm.sup.2 of the microprojection array in 10 msec or less.

The preferred reservoir useful with the present invention is in the form of a solid coating directly on the surfaces of the microprojections. Preferably, the coating is applied in a liquid state and then dried. The volatile liquid solution or suspension containing the antigenic agent and adjuvant can be applied to the microprojection array by immersion, spraying and/or other known microfluidic dispensing techniques. Thereafter, the coating is allowed to dry to form a solid antigen and adjuvant-containing coating. Preferably, only those portions of the microprojection array which penetrate into the skin tissue are coated with the antigenic agent. Suitable microprojection coating methods and apparatus are disclosed in Trautman et al. U.S. patent application Ser. No. 10/099,604 filed Mar. 15, 2002, the disclosures of which are incorporated herein by reference. Using the coating methods disclosed therein and the coating compositions disclosed herein, we have been able to precisely and uniformly coat only the tips of the skin piercing microprojections in typical metal (i.e., titanium) microprojection arrays having microprojection lengths of less than 500.mu.m.

While the relative amounts of adjuvant and antigenic agent delivered intradermally in accordance with the present invention will vary depending upon the particular antigenic agent and adjuvant being delivered, typically the weight ratio of delivered adjuvant to delivered antigen should be in the range of about 0:5 to 50:1 and more preferably in the range of about 1:1 to 10:1. In order to achieve these adjuvant-to-antigenic-agent delivery ratios, the reservoir preferably contains loadings of the antigenic agent and the immune response augmenting adjuvant in the same weight ratios disclosed immediately above.

Furthermore, with microprojection tip coating, therapeutic compound loadings of at least 0.2.mu.g per cm.sup.2 of the microprojection array, and preferably at least 2.mu.g per cm.sup.2 of the array are easily achieved. For a typical 5 cm.sup.2 array, this translates into antigenic agent and adjuvant loadings of at least 1.mu.g, and preferably at least 10.mu.g, which is more than adequate for most vaccinations. With microprojection tip coating of the antigenic agent and adjuvant, the delivery efficiency (E.sub.del) is greatly enhanced. E.sub.del is defined as the percent, by weight, of the antigenic agent and adjuvant released from the coating per predetermined period of time. With tip coating of the antigenic agent and adjuvant-containing solutions or suspensions, an E.sub.del of at least 30% in 1 hour, and preferably at least 50% in 15 minutes can be achieved. Thus, the present invention offers significant cost advantages over conventional macrotine skin piercing devices used in the prior art.

The present invention can be used to deliver transdermally by electrotransport the therapeutic compounds of the present invention.

Generally speaking, it is most preferable to use a water soluble form of the drug or agent to be delivered. Drug or agent precursors, i.e., species which generate the selected species by physical or chemical processes such as ionization, dissociation, dissolution or covalent chemical modification (i.e., prodrugs), are within the definition of “agent” or “drug” herein. “Drug” or “agent” is to be understood to include charged and uncharged species as described above.

While the disclosure has focused upon the electrotransport delivery of ionic species, the present invention is also applicable to the electrotransport delivery of uncharged species, e.g., by electroosmosis. Thus, the transformation of the skin into the high efficiency transport state is not limited to electrically assisted transport of ionic species but also to electroosmotic delivery of uncharged (i.e., non-ionized) species.

It is an aspect of this invention to provide sustained release formulations to administer a therapeutically effective amount of AICA riboside and analogs thereof and/or its active metabolites, over an administration period. More specifically, it is an aspect of this invention to provide compositions and methods for the transdermal delivery of AICA riboside and analogs thereof and/or its active metabolites, and delivery systems for effecting the same, which are suitable for the transdermal administration of AICA riboside and analogs thereof and/or its active metabolites continuously through a body surface or membrane at a therapeutically effective rate in order to achieve and maintain therapeutic blood or plasma levels in an individual.

Another aspect of this invention is to improve the compliance of patients in need of AICA riboside and analogs thereof therapy by providing compositions, devices, and methods for the transdermal administration of AICA riboside and analogs thereof at a therapeutically effective rate.

According to this invention, it has been discovered that AICA riboside and analogs thereof can be safely and efficaciously administered transdermally at a therapeutically effective rate to provide, among other things, treatment for hypertension, congestive heart failure, and acute renal failure when coadministered with a suitable permeation enhancer. Therefore, the invention comprises the following aspects, either alone or in combination:

A composition of matter for the transdermal administration of AICA riboside and analogs thereof comprising an amount of AICA riboside and analogs thereof and a permeation enhancer in a carrier effective to permit sustained release of AICA riboside and analogs thereof at a therapeutically effective rate during an administration period in order to administer a therapeutically effective amount of AICA riboside and analogs thereof to achieve and maintain therapeutic blood or plasma levels throughout a substantial portion of the administration period.

A device for the transdermal administration of AICA riboside and analogs thereof at a therapeutically effective rate, comprising: (a) a reservoir comprising AICA riboside and analogs thereof and a permeation-enhancing amount of a permeation enhancer; (b) a backing behind the body contacting-distal surface of the reservoir; and (c) means for maintaining the reservoir in AICA riboside and analogs thereof transmitting relation with the skin, wherein a therapeutically effective amount of AICA riboside and analogs thereof is delivered at a therapeutically effective rate during an administration period in order to achieve and maintain therapeutic blood or plasma levels throughout a substantial portion of the administration period.

The permeation enhancer may be any permeation enhancer known in the art to increase permeability of drugs through skin and includes, but is not limited to, those disclosed in the above cited patents. Preferably, the permeation enhancer comprises a permeation enhancing amount of a permeation enhancer including, but not limited to monoglycerides, C.sub.10-C.sub.20 fatty acid esters including ethyl palmitate and isopropyl myristate; acyl lactylates such as caproyl lactylic acid and lauroyl lactylic acid; dimethyl lauramide; dodecyl (lauryl)acetate; lactate esters such as lauryl lactate, and myristyl lactate; monoalkyl ethers of polyethyleneglycol and their alkyl or aryl carboxylic acid esters and carboxymethyl ethers such as polyethylene glycol-4 lauryl ether (Laureth-4) and polyethylene glycol-2 lauryl ether (Laureth-2); Myreth-3, myristyl sarcosine, and methyl laurate.

Additionally, the invention is directed to a method for treating an individual suffering from hypertension, congestive heart failure, and/or acute or chronic renal failure comprising transdermally administering AICA riboside and analogs thereof to the individual wherein a therapeutically effective amount of AICA riboside and analogs thereof is delivered at a therapeutically effective rate during an administration period in order to achieve and maintain therapeutic blood or plasma levels of AICA riboside and analogs thereof throughout a substantial portion of the administration period. According to this invention, it has been discovered that AICA riboside and analogs thereof can be safely and efficaciously administered by a sustained release formulation. More specifically, it has been found that AICA riboside and analogs thereof can be safely and efficaciously administered transdermally at a therapeutically effective rate to provide, among other things, treatment for hypertension, congestive heart failure, and acute renal failure when coadministered with a suitable permeation enhancer. The present invention provides novel compositions, devices, and methods for AICA riboside and analogs thereof therapy with improved patient compliance to an individual in need of such therapy.

Therapeutic blood or plasma levels can be obtained from administration rates in the range of 20-1500.mu.g/hr, preferably about 60-1000.mu.g/hr. Representative in vitro skin fluxes of AICA riboside and analogs thereof through human skin are in the range of about 5 ng/cm.sup.2.multidot.hr-5.5.mu.g/cm.sup.2.multidot.hr, depending on the drug form, permeation enhancer, and adhesive.

This invention finds particular usefulness in administering AICA riboside and analogs thereof across skin. According to the invention, AICA riboside and analogs thereof is placed in AICA riboside and analogs thereof transmitting relationship to the skin, preferably in a pharmaceutically acceptable carrier thereof, and maintained in place for the desired administration period.

The AICA riboside and analogs thereof and permeation enhancer are typically dispersed within a physiologically compatible matrix or carrier, as more fully described below, which may be applied directly to the body as an ointment, gel or cream. When used in the form of a liquid, ointment, lotion, cream or gel applied directly to the skin, it is preferable, although not required, to occlude the site of administration. Such compositions can also contain other permeation enhancers, stabilizers, dyes, diluents, pigments, vehicles, inert fillers, anti-irritants, excipients, gelling agents, vasoconstrictors, vasodilators, and other components of topical compositions as are known to the art.

In other embodiments, AICA riboside and analogs thereof would be administered from a transdermal delivery device.

AICA riboside and analogs thereof can be administered to human skin by direct application to the skin in the form of an ointment, gel, cream or lotion, for example, but are preferably administered from a skin patch or other known transdermal delivery device which contains a saturated or unsaturated formulation of the AICA riboside and analogs thereof and enhancer. The formulation may be aqueous or non-aqueous. The formulation should be designed to deliver the AICA riboside and analogs thereof and any anti-irritant and/or enhancer at the necessary fluxes. Aqueous formulations typically comprise water or water/ethanol and about 1-5 wt % of a gelling agent, an example being a hydrophilic polymer such as hydroxyethylcellulose or hydroxypropylcellulose. When using aqueous formulations, it is preferable to maintain the pH at less than about 5.5, more preferably between about pH 2-4.5 in order to provide a stable AICA riboside and analogs thereof formulation. Typical non-aqueous gels are comprised of silicone fluid or mineral oil. Mineral oil-based gels also typically contain 1-2 wt % of a gelling agent such as colloidal silicon dioxide. The suitability of a particular gel depends upon the compatibility of its constituents with the AICA riboside and analogs thereof, anti-irritant, and the permeation enhancer in addition to any other components in the formulation.

The reservoir matrix should be compatible with AICA riboside and analogs thereof, the permeation enhancer, and any carrier therefor. The term “matrix” as used herein refers to a well-mixed composite of ingredients. When using an aqueous formulation, the reservoir matrix is preferably a hydrophilic polymer, e.g., a hydrogel.

When using a non-aqueous formulation, the reservoir matrix is preferably composed of a hydrophobic polymer. Suitable polymeric matrices are well known in the transdermal drug delivery art, and examples are listed in the above-named patents previously incorporated herein by reference. A typical laminated system would consist essentially of a polymeric membrane and/or matrix such as ethylene vinyl acetate (EVA) copolymers, such as those described in U.S. Pat. No. 4,144,317, preferably having a vinyl acetate (VA) content in the range of from about 9% up to about 60% and more preferably about 9% to 40% VA. Polyisobutylene/oil polymers containing from 4-25% high molecular weight polyisobutylene and 20-81% low molecular weight polyisobutylene with the balance being an oil such as mineral oil or polybutene may also be used as the matrix material.

The amount of AICA riboside and analogs thereof present in the therapeutic device and required to achieve an effective therapeutic result depends on many factors, such as the minimum necessary dosage of the AICA riboside and analogs thereof for the particular indication being treated; the solubility and permeability of the matrix, taking into account the presence of permeation enhancer, of the adhesive layer and of the rate-controlling membrane, if present; and the period of time for which the device will be fixed to the skin. The minimum amount of AICA riboside and analogs thereof is determined by the requirement that sufficient quantities of AICA riboside and analogs thereof must be present in the device to maintain the desired rate of release over the given period of application.

The AICA riboside and analogs thereof may be present in the matrix or carrier at a concentration at or below saturation. An excess amount of AICA riboside and analogs thereof above saturation may be included in the matrix or carrier, the amount of excess being a function of the desired length of the delivery period of the system. AICA riboside and analogs thereof may be present at a level below saturation without departing from this invention as long as it is continuously administered to the skin site at a therapeutic rate and for a period of time sufficient to deliver a therapeutically effective amount of AICA riboside and analogs thereof that provides the desired therapeutic result.

The permeation enhancer useful in the present invention is selected from those compounds which are compatible with AICA riboside and analogs thereof and which provide enhanced skin permeation to the drug when it is administered together with the drug to the skin of a user. Additionally, the permeation enhancer must not adversely interact with the adhesive of the in-line contact adhesive layer if one is present. Examples of permeation enhancers are disclosed in the patents cited above previously incorporated by reference and can be selected from, but are not limited to, fatty acids, monoglycerides of fatty acids such as glycerol monolaurate, glycerol monooleate, glycerol monocaprate, glycerol monocaprylate, or glycerol monolinoleate; lactate esters of fatty acids such as lauryl lactate, cetyl lactate, and myristyl lactate; acyl lactylates such as caproyl lactylic acid; esters of fatty acids having from about 10 to about 20 carbon atoms, including, but not limited to, isopropyl myristate, and ethyl palmitate; alkyl laurates such as methyl laurate; dimethyl lauramide; lauryl acetate; monoalkyl ethers of polyethyleneglycol and their alkyl or aryl carboxylic acid esters and carboxymethyl ethers such as polyethylene glycol-4 lauryl ether (Laureth-4) and polyethylene glycol-2 lauryl ether (Laureth-2); polyethylene glycol monolaurate; myristyl sarcosine; Myreth-3; and lower C.sub.1-4 alcohols such as isopropanol and ethanol, alone or in combinations of one or more.

A preferred permeation enhancer according to this invention comprises a monoglyceride of a fatty acid together with a suitable cosolvent, including, but not limited to, lauryl acetate as disclosed in WO 96/40259 and esters of C.sub.10-C.sub.20 fatty acids such as lauryl lactate, ethyl palmitate, and methyl laurate. Ethyl palmitate has been found to be particularly desirable as it is obtainable at a high degree of purity, thus providing a purer and better defined permeation enhancer and a system which is more readily characterized. According to a particularly preferred embodiment, the permeation enhancer comprises glycerol monolaurate (GML) and ethyl palmitate within the range of 1-25 wt % and 1-20 wt %, respectively, at a ratio of GML/ethyl palmitate within the range of 0.5-5.0, preferably 1.0-3.5. A particularly preferred embodiment comprises 20 wt % GML and 12 wt % ethyl palmitate.

Another embodiment is directed to the use of surfactant sarcosines, preferably myristyl sarcosine, as a permeation enhancer for pharmaceutically acceptable salts of AICA riboside and analogs thereof, preferably AICA riboside and analogs thereof mesylate. In general, formulations comprising AICA riboside and analogs thereof base were found to be more permeable through the skin as compared to formulations comprising pharmaceutically acceptable salts of AICA riboside and analogs thereof such as AICA riboside and analogs thereof mesylate. However, formulations comprising myristyl sarcosine as a permeation enhancer for AICA riboside and analogs thereof mesylate were found to exhibit higher transdermal fluxes than formulations of the base. Additionally, AICA riboside and analogs thereof mesylate when administered transdermally did not exhibit the long lag period observed with AICA riboside and analogs thereof base. Thus, according to this embodiment, transdermal compositions, devices, and methods are provided comprising a pharmaceutically acceptable salt of AICA riboside and analogs thereof, preferably AICA riboside and analogs thereof mesylate, together with a permeation enhancer for the AICA riboside and analogs thereof salt, preferably myristyl sarcosine, in order to transdermally administer AICA riboside and analogs thereof at therapeutically effective rates and lowered lag time. The use of other surfactant sarcosines such as lauroyl sarcosine, sodium lauryl sarcosine, cocoyl sarcosine, and oleoyl sarcosine are contemplated for use with the compositions, devices, and methods according to this embodiment.

The permeation-enhancing mixture is dispersed through the matrix or carrier, preferably at a concentration sufficient to provide permeation-enhancing amounts of enhancer in the reservoir throughout the anticipated administration period. Where there is an additional, separate permeation enhancer matrix layer as well, as in FIGS. 3 and 4, the permeation enhancer normally is present in the separate reservoir in excess of saturation.

According to another preferred embodiment, an anti-irritant is dispersed throughout the matrix or carrier, preferably at a concentration sufficient to deliver anti-irritant to the skin in an amount effective to reduce skin irritation throughout the anticipated administration period. The anti-irritant is preferably present in excess of saturation in order to ensure that the anti-irritant is continuously administered with the AICA riboside and analogs thereof and continues to be present as long as any AICA riboside and analogs thereof is present in the epidermis. Suitable anti-irritants include, but are not limited to, methyl nicotinate as disclosed in U.S. Pat. No. 5,451,407, corticosteroids, and buffering agents including ascorbic acid and acetic acid. Such anti-irritants are known in the art as seen in the above cited patents previously incorporated by reference.

For example, if a corticosteroid is used as the anti-irritant, it is preferably administered at a flux within the range of 0.1-5.0.mu.g/cm.sup.2.multidot.hr. Hydrocortisone is a preferred corticosteroid and is present in an amount of about 1-5 wt %. The total amount of hydrocortisone administered is not to exceed 5 mg/24 hour in order to avoid possible systemic effects. Hydrocortisone esters such as hydrocortisone acetate are also suitable. More potent corticosteroids may not require a permeation enhancer as hydrocortisone and hydrocortisone acetate do. However, the advantages of hydrocortisone or its esters such as hydrocortisone acetate is that they are approved for over-the-counter use. This invention contemplates the use of any corticosteroid in addition to hydrocortisone and includes, without limitation, beclomethasone, betamethasone, benzoid, betamethasone dipropionate, betamethasone valerate, clobetasol propionate, clobetasol butyrate, desonide, dexamethasone, fluocinonide, prednisolone, and triamcinolone, for example.

Because of the wide variation in skin permeability from individual to individual and from site to site on the same body, it may be preferable that the AICA riboside and analogs thereof, anti-irritant, and/or permeation enhancer, be administered from a rate-controlled transdermal delivery device. Rate control can be obtained either through a rate-controlling membrane as described in U.S. Pat. No. 3,797,494 listed above, or through an adhesive or both as well as through other means known in the art.

A certain amount of AICA riboside and analogs thereof may bind reversibly to the skin, and it is accordingly preferred that the skin-contacting layer of the device include this amount of AICA riboside and analogs thereof as a loading dose.

The surface area of the device of this invention can vary from about 1-200 cm.sup.2. A typical device, however, will have a surface area within the range of about 5-60 cm.sup.2, preferably about 20 cm.sup.2.

The devices of this invention can be designed to deliver AICA riboside and analogs thereof effectively for an extended time period of from several hours up to 7 days or longer. Seven days is generally the maximum time limit for application of a single device because the adverse effects of occlusion of a skin site increases with time and the normal cycle of sloughing and replacement of the skin cells occurs in about 7 days.

Preferably, a device for the transdermal administration of AICA riboside and analogs thereof, at a therapeutically effective rate, comprises:

(a) a reservoir comprising: (i) 1-50% by weight AICA riboside and analogs thereof, (ii) 1-50% by weight of a permeation enhancer, (iii) 30 to 90% by weight of a polymeric carrier;

(b) a backing behind the skin-distal surface of the reservoir; and

(c) means for maintaining the reservoir in AICA riboside and analogs thereof—transmitting relation with the skin.

More preferably, a device for the transdermal administration of AICA riboside and analogs thereof, at a therapeutically effective rate, comprises:

(a) a reservoir comprising: (i) 1-50% by weight AICA riboside and analogs thereof, (ii) 5-40% by weight of a permeation enhancer, (iii) 30-90% by weight of a polymeric carrier;

(b) a backing behind the skin-distal surface of the reservoir; and

(c) means for maintaining the reservoir in AICA riboside and analogs thereof—transmitting relation with the skin.

Most preferably, a device for the transdermal administration of AICA riboside and analogs thereof, at a therapeutically effective rate, comprises:

(a) a reservoir comprising: (i) 5-50% by weight AICA riboside and analogs thereof, (ii) 5-40% by weight of a permeation enhancer comprising a monoglyceride and a fatty acid ester, (iii) 30-90% by weight of a polymeric carrier;

(b) a backing behind the skin-distal surface of the reservoir; and

(c) means for maintaining the reservoir in AICA riboside and analogs thereof—transmitting relation with the skin.

The backing may be flexible or nonflexible and may be a breathable or occlusive material. Suitable materials include, without limitation, polyethylene, polyurethane, polyester, ethylene vinyl acetate, acrylonitrile, cellophane, cellulose acetate, cellulosics, ethylcellulose, ethylene vinyl alcohol, plasticized vinylacetate-vinylchloride copolymers, polyethylene terephthalate, nylons, rayon, polypropylene, polyvinyl alcohol, polyvinyl chloride, metalized polyester films, polyvinylidene chloride, polycarbonate, polystyrene, and aluminum foil. The backing may be a multi-laminate film.

The means for maintaining the reservoir in drug and permeation enhancer transmitting relation with the skin is preferably a pressure sensitive adhesive including, but not limited to, polyisobutylene adhesives, silicone adhesives, and acrylate adhesives known in the art including copolymers and graft copolymers thereof. A further embodiment of the invention is directed to including in the adhesive a small percentage, e.g., from about 1 to about 5 wt % of AICA riboside and analogs thereof to assure an appropriate initial release rate.

The aforementioned patents describe a wide variety of materials which can be used for fabricating various layers or components of the transdermal AICA riboside and analogs thereof delivery systems according to this invention. This invention, therefore, contemplates the use of materials other than those specifically disclosed herein including those which may become hereafter known to the artist capable of performing the necessary functions.

The invention is also directed to a method of continuously administering AICA riboside and analogs thereof to a patient at a therapeutically effective rate over an administration period in order to administer a therapeutically effective amount and achieve and maintain therapeutic blood or plasma levels in a patient.

The length of time of AICA riboside and analogs thereof presence and the total amount of AICA riboside and analogs thereof in the plasma can be changed following the teachings of this invention to provide different treatment regimens. Thus, they can be controlled by the amount of time during which exogenous AICA riboside and analogs thereof is delivered transdermally to an individual or animal and the rate at which it is administered.

### EXAMPLES

The following examples describes specific aspects of the invention to illustrate the invention and provide a description of the methods, compositions, and formulations of the invention. The examples should not be construed as limiting the invention, as the example merely provides specific methodology useful in understanding and practicing the invention.

### Example A

### Preparation Of 5-Amino-(2,3,5-tri-O-acetyl-beta-D-ribofuranosyl)imidazole-4-carboxamide (Compound No. 2 (I-III))

AICA riboside (50 g) was dissolved in pyridine (450 ml) and then cooled in an ice bath. Acetic anhydride (80 ml) was added and the ice bath removed. The reaction mixture was stirred for 3 hrs. TLC on silica gel, eluting with 9:1 methylene chloride:methanol, showed the reaction to be complete. Methanol (5 ml) was added to neutralize unreacted acetic anhydride. The solvents were removed by evaporation under high vacuum (bath temperature less than 40° C.). The residue was coevaporated with dimethylformamide (3×150 ml). The residue was crystallized from ethanol using seed crystals. The yield of the triacetate 62 g of white solid; melting point 128°-129° C.

NMR (DMSO-d₆) Δ ppm 2.05-2.15 (2s, 9H, —CH₃), 4.3 (broad s, 3H, 4′-CH, 5′-CH₂), 5.3 (m, 1H, 3′-CH) 5.55 (t, 1H, 2′-CH), 5.87 (d, 1H, 1′-CH), 5.9 (broad s, 2H, 5-NH₂), 6.7-6.9 (broad d, 2H, 4-NH₂), 7.4 (s, 1H, 2-CH).

The preparation this compound is also described in U.S. Pat. No. 3,450,693 to K. Suzuki & I. Kumoshiro (1969); See also Chem. Abs. 71: 816982 (1969).

### Example B

### Preparation of N⁵-dimethylaminomethyleneamino-beta-D-ribofuranosylimidazole-4-carboxamide (Compound No. 7 (1-164))

Dissolved 2′,3′,5′-tri-O-acetyl AICA riboside (10 g) in dimethylformamide (30 ml) and dimethylformamide dimethyl acetal (20 ml). The reaction mixture was allowed to stir overnight. TLC on silica gel, eluting with 9:1 methylene chloride:methanol, showed that the reaction was complete by absence of starting material. The solvent was removed by evaporation under high vacuum (bath temperature less than 40° C.). The residue was dissolved in cyclohexylamine and stirred overnight. The solvent was removed by evaporation under reduced pressure and the residue was crystallized from ethanol. Yield was 4.6 g of white solid, melting point 173°-175° C.

NMR (MeOH-d₄), Δ ppm 3.0-3.05 (2s, 6H, N(CH₃)₂), 3.75 (m, 2H, 5′-CH₂), 4.0 (g, 1H, 4′-CH), 4.2 (t, 1H, 3′-CH), 4.35 (t, 1H, 2′-CH), 5.8 (d, 1H, 1′-CH), 7.7 (s, 1H, 2-CH), 8.25 (s, 1H, 5-N═CH—N)

### Example C

### Preparation of 5-Amino-1-beta-D-ribofuranosylimidazole-4-N-(cyclopentyl)carboxamide (Compound No. 10 (1-186))

The literature procedure of P. C. Srivastava, R. W. Mancuso, R. J. Rosseau and R. K. Robins, J. Med. Chem. 17(11), 1207 (1977) was followed to synthesize N-succinimidyl-5-amino-1-(2,3,5-tri-O-acetyl-β-D-ribofuranosyl)imidazole-4-carboxylate (“intermediate No. 4”). Intermediate No. 4 (3.9 g) was dissolved in methylene chloride (60 ml). Cyclopentylamine (0.8 ml) was added and the solution was stirred overnight. TLC on silica, eluting with 9:1 methylene chloride:methanol, showed the reaction was complete by absence of starting material. The solvent mixture was extracted with 5% hydrochloric acid solution (100 ml), saturated sodium bicarbonate solution (100 ml) and water (200 ml). The organic layer was dried over sodium sulfate and evaporated under reduced pressure to give 3.1 g of yellow foam. The acetyl groups were removed by dissolving the 3.1 g of foam in methanol (70 ml) and cooling in an ice bath. Ammonium hydroxide (60 ml) was added and the ice bath was removed. After 2½ hours stirring, TLC on silica gel, eluting with 9:1 methylene chloride:methanol, showed all starting material was gone. The solvent was evaporated under reduced pressure to give a residue which was purified on a silica column, eluting with 9:1 and 6:1 methylene chloride:methanol. Fractions which were alike by TLC were pooled and evaporated under reduced pressure to yield 1.1 g of white foam crystallized from methanol-ethyl acetate, melting point 158°-160° C.

NMR (DMSO-d₆), Δ ppm 1.4-1.9 (m, 8H, —CH₂—CH₂—), 3.6 (m, 2H, 5′-CH₂), 3.9 (d, 1H, NH—CH), 4.0-4.35 (m, 3H, 2′,3′,4′-CH), 5.15-5.4 (m, 3H, 2′,3′,5′-OH), 5.45 (d, 1H, 1′-CH), 5.9 (broad s, 2H, —NH₂), 7.1 (d, 1H, —NH—), 7.3 (s, 1H, 2-CH).

### Example D

### Preparation of 5-Amino-1-beta-D-ribofuranosylimidazole-4-N-(cyclopropyl)carboxamide (Compound No. 12 (1-232))

This compound was prepared following the procedure described in Example C except cyclopropylamine (0.5 ml) was substituted for cyclopentylamine (0.8 ml). The yield starting with 6.2 g of intermediate No. 4 (the succinate ester) was 2.3 g.

NMR (DMSO-d₆) Δ ppm 0.5 (m, 4H, CH₂—CH₂) 2.7 (m, 1H, N—CH), 3.6 (m, 2H, 5′-CH₂), 3.8-4.3 (m, 3H, 2′,3′,4′-CH), 5.15-5.4 (m, 3H, 2′,3′,5′-OH) 5.45 (d, 1H, 1′-CH), 5.9 (s, 2H, NH₂), 7.2 (s, 1H, 2-CH) 7.4 (d, 1H, 4-NH).

### Example E

### Preparation of 5-Amino-1-beta-D-ribofuranosylimidazole-4-N-(benzyl)carboxamide (Compound No. 11 (1-226))

Inosine (10 g) was suspended in dimethylformamide (100 ml) and dimethylformamidedibenzylacetal (25 ml). The resulting mixture was stirred at 70° C. overnight. TLC on silica, eluting with 6:1 methylene chloride:methanol, showed completion of reaction. Solvent was removed by evaporation at reduced pressure. The remainder was dissolved in ammonium hydroxide (130 ml). The mixture was stirred overnight, then evaporated under reduced pressure. Ethanol (80 ml) was added to the residue and the resulting mixture was warmed. The solid was collected by filtration. Yield of 1-benzylinosine was 10.5 g which was characterized by NMR.

The intermediate, 1-benzylinosine (10.5 g), was dissolved in ethanol (1.0 L) and 3M sodium hydroxide solution (140 ml). This solution was refluxed for 3 hours. TLC on silica showed the reaction was complete. The solvent was removed by evaporation under reduced pressure. The residue was chromatographed on a silica gel column, eluting with 6:1 methylene chloride:methanol. Fractions were collected which were similar by TLC and concentrated until crystals appeared. Yield was 7.4 g of the above-identified compound as a white solid, melting point 178°-179° C.

NMR (DMSO-d₆) Δ ppm 3.6 (m, 2H, 5′-CH₂) 3.85-4.35 (m, 3H, 2′,1′,3′,4′-CH), 4.4 (d, 2H, N—CH₂), 5.15-5.4 (m, 3H, 2′,3′,5′-OH), 5.5 (d, 1H, 1′-CH), 5.9 (broad s, 2H, 5-NH₂), 7.2-7.4 (m, 6H, 2-CH, C₆H₅) 7.95 (t, 1H, NH). See also E. Shaw, J.A.C.S. 80: 3899 (1958).

### Example F

### Preparation of 5-Amino-1-β-D-ribofuranosylimidazole-4-carboxylic acid methyl ester (Compound No. 14 (1-260))

5-amino-1-(2,3,5-tri-O-acetyl-β-D-ribofuranosyl)-imidazole-4-carboxylic acid (3.85 g, 10 mmol) was dissolved in 40 ml tetrahydrofuran and cooled to 0° C. An excess of diazomethane in ether was added and the mixture warmed to room temperature. Acetic acid was added to destroy excess diazomethane and the mixture was evaporated to dryness. The residue was purified by chromatography on silica gel, eluting with 7:3 ethyl acetate:hexane. The major product fractions, judged by silica thin layer chromatography (TLC) using the above system, were combined and evaporated to yield 1.2 g of a white foam. This was dissolved in 40 ml of methanol containing 20 mg of sodium methoxide and stirred for 30 minutes. Silica TLC, eluting with 6:1 methylene chloride:methanol, showed no remaining starting material and a new slower-moving product spot. The reaction was neutralized with Dowex 50 (H⁺) resin and evaporated to yield 0.64 g of the desired product as a white foam. IR (KBr): 1725 cm⁻¹ (—CO—OCH₃).

NMR (DMSO-d₆): Δ ppm, 3.65 (s, 3H, CH₃), 3.8 (m, 3H, 4′-CH and 5′-CH₂), 4.1 (m, 1H, 3′-CH), 4.2 (m, 1H, 2′-CH), 5.5 (d, 1H, 1′-CH), 8.0 (s, 1H, 2-CH).

### Example G

### Preparation of 5-Amino-5′-sulfamoyl-1-β-D-ribofuranosyl-imidazole-4-carboxamide (Compound No. 15 (1-261))

### A. Preparation of 5-Amino-2′,3′-isopropylidene-1-β-ribofuranosyl-5-sulfamoylimidazole-4-carboxamide

To a solution of 2′,3′-isopropylidene-AICA-riboside (2.98 g, 10 mmol) in dry N,N-dimethylformamide (25 ml), sodium hydride (300 mg, 80% dispersion in oil) was added over a period of 10 min. After the evolution of hydrogen gas had ceased, the flask was immersed in an ice bath and the mixture was stirred for 30 min. A solution of sulfamoyl chloride (1.3 g, 11 mmol) in dry tetrahydrofuran (20 ml) was added slowly. TLC of the reaction mixture (silica gel, solvent 9:1 methylene chloride:methanol) indicated presence of some starting material. An additional 200 mg of sulfamoyl chloride in tetrahydrofuran (10 ml) was added and the resulting mixture stirred for one hour. Methanol (1 ml) was added and solvent was evaporated under high vacuum. The residue chromatographed over silica gel, eluting with a mixture of methylene chloride:methanol (9:1). Several fractions were collected. Fractions showing identical TLC patterns were pooled and evaporated to a glassy product. Yield was 1.5 g.

¹H-NMR (DMSO-d₆) Δ ppm, 1.25 and 1.55 (2s, 6H, C(CH₃)₂), 4.1 (d, 2H, 5′-CH₂), 4.25-4.35 (m, 1H, 4′-CH), 4.8-4.9 and 5.1-5.2 (2m, 2H, 2′-CH and 3′-CH), 5.8 (d, 1H, 1′-CH), 5.9 (s, 2H, 5-NH₂), 6.65-6.95 (br. d, 2H, CONH₂), 7.35 (s, 1H, 2-CH), 7.7 (s, 2H, SO₂ NH). The NMR data conformed to the structure of 5-amino-2′,3′-isopropylidene-1-β-ribofuranosyl-5′-sulfamoylimidazole-4-carboxamide. This intermediate product was used in the following deblocking step without further purification or isolation.

### B. Preparation of 5-Amino-5′-sulfamoyl-1-β-D-ribofuranosyl-imidazole-4-carboxamide

(Compound No. 15 (1-261))

The compound from the preceeding preparation was dissolved in 60% formic acid (20 ml) and the resulting solution was stirred at room temperature for 48 hours. The solvent was removed by evaporation under high vacuum. The residue was coevaporated with water. The product was crystallized from aqueous ethanol. Yield was 1.0 g of the above-identified product, melting point 174°-175° C.

¹H-NMR (DMSO-d₆) Δ ppm 3.9-4.3 (m, 5H, 2′-CH, 3′-CH, 4′-CH and 5′-CH₂), 5.4 and 5.5 (2d, 2H, 2′-OH and 3′-OH), 5.5 (d, 1H, 1′-CH), 5.8 (br.s, 2H, 5-NH₂), 6.6-6.9 (br.d, 2H, CONH₂), 7.3 (s, 1H, 2-CH) and 7.6 (s, 2H, SO₂ NH₂).

### Example H

### Preparation of 5′-Amino-5′-deoxy-AICA-riboside (Compound No. 21 (1-227))

### A. Preparation of 5′-Azido-5′-deoxy-AICA-riboside

A mixture 5′-deoxy-5′-iodo-2′,3′-isopropylidene-AICA riboside (8.0 g) (Ref: P. C. Srivastava, A. R. Newman, T. R. Mathews, and R. K. Robins, J. Med. Chem. 18 1237 (1975)), lithium azide (4.0 g), and N,N-dimethylformamide was heated at 80°-90° C. for 5 hours. The mixture was evaporated to dryness and the residue was chromatographed over silica gel column eluting with methylene chloride. The fast moving product-containing fractions were pooled and evaporated to obtain 7.2 g of a product which was subjected to deblocking with 60% formic acid (100 ml) at room temperature for 48 hours. Excess formic acid was removed by evaporation under high vacuum. The residue was coevaporated with water (3×25 ml) to obtain a semi-solid product. This product was crystallized from aqueous ethanol. Yield was 5.0 g, of the above-identified product, melting point 138°-139° C. ¹H NMR (DMSO-d₆) Δ ppm 3.55 (d, 2H, 5′-CH₂), 3.95 (br. s, 2H, 3′-CH and 4′-CH), 4.2-4.4 (m, 1H, 2′-CH), 5.35 and 5.50 (2d, 2H, 2′-OH and 3′-OH), 5.55 (d, 1H, 1′-CH), 5.75-5.9 (br. s, 2H, 5-NH₂), 6.6-6.9 (br. d, 2H, CONH₂) and 7.35 (s, 1H, 2-CH). IR (KBr) cm⁻¹: 3400-3000 (br. NH₂, CONH₂, OH, etc.), 2150 (S, N₃) 1640 (CONH₂).

### B. Preparation of 5′-Amino-5′-deoxy-AICA-riboside

A solution of 5′-azido-5′-deoxy-AICA-riboside (800 mg) (the product of step (A)) in methanol (40 ml) was hydrogenated in a Parr apparatus with palladium on carbon (5%) (100 mg) as the hydrogenation catalyst at 40 psi for 60 min. The catalyst was removed by filtration of the reaction mixture through a celite pad. The clear filtrate was evaporated to dryness. The product was crystallized from boiling ethanol. Yield was 650 mg of the above-identified product, melting point 188°-189° C. ¹H-NMR (D₂O) Δ ppm, 2.7 (d, 2H, 5′-CH₂), 3.8-4.4 (3m, 3H, 2′-CH, 3′-CH and 4′-CH), 5.4 (d, 1H, 1′-CH) and 7.3 (s, 1H, 2-CH). IR (KBr) cm⁻¹: 3500-3000 (br. OH, NH₂, CONH₂, etc.), 1640-1645 (br.s. CONH₂).

### Example I

### Preparation of 5-Amino-1-(2-O-methyl-β-D-ribofuranosyl)-imidazole-4-carboxamide (Compound No. 20 (1-188)) and 5-Amino-1-(3-O-methyl-β-D-ribofuranosyl)imidazole-4-carboxamide (Compound No. 22 (1-243))

5-Amino-1-β-D-ribofuranosylimidazole-4-carboxamide (5.2 g, 20 mmol) was dissolved in 40 ml hot dimethylformamide and diluted with 70 ml methanol containing 35 mg tin(II) chloride dihydrate. A solution of 0.1 mol of diazomethane in 200 ml of ether was added in portions over 45 min. After each addition, 20 mg of tin(II) chloride dihydrate was added. The resulting mixture was filtered and evaporated to give a syrup. The syrup was dissolved in 25 ml of methanol and upon cooling yielded crystalline 5-amino-1-(2-O-methyl-β-D-ribofuranosyl)imidazole-4-carboxamide which was collected by filtration and dried. Yield was 1.2 g, melting point 1140-117° C. ¹H NMR (DMSO-d₆) (for Compound 20): Δ ppm, 3.3 (s, 3H, CH₃), 3.6 (m, 2H, 5′-CH₂), 3.9 (m, 1H, 4′-CH), 4.1 (m, 1H, 2′-CH), 4.2 (m, 1H, 3′-CH), 5.2 (d, 1H, 3′-OH), 5.3 (t, 1H, 5′-OH), 5.6 (d, 1H, 1′-CH), 6.0 (br. s, 2H, 5-NH₂), 6.7 (br. d, 2H, 4-CONH₂), 7.3 (s, 1H, 2-CH).

The supernatant from the above crystallization was concentrated and applied to a 200 ml column of silica gel. The column was eluted with 10:1 methylene chloride:methanol (1 L), 8:1 methylene chloride:methanol (500 ml) and 5:1 methylene chloride:methanol (500 ml). The 5:1 eluate contained a major product and was evaporated and residue dissolved in 10 ml of methanol. Upon cooling this yielded crystals which were collected and dried. Yield was 1.4 grams. By NMR decoupling and exchange experiments the product was shown to be 5-amino-1-(3-O-methyl-β-D-ribofuranosyl)imidazole-4-carboxamide. ¹H NMR (DMSO-d₆) (for Compound 18): Δ ppm: 3.3 (s, 3H, CH₃), 3.6 (m, 2H, 5′-CH₂), 3.7 (m, 1H, 4′-CH), 4.0 (m, 1H, 3′-CH), 4.4 (m, 1H, 2′-CH), 5.3 (t, 1H, 5′-OH), 5.4 (2d, 2H, 2′-CH and 1′-CH), 5.9 (br. s, 2H, 5-NH₂), 6.7 (br. d, 2H, CO—NH₂), 7.7 (s, 1H, 2-CH).

### Example J

### Preparation of 5-Amino-1-β-D-ribofuranosyl-imidazole-4-N->(4-nitrophenyl) methyl!carboxamide (Compound No. 23 (1-343))

N-Succinimidyl-5-amino-1-(2,3,5-tri-O-acetyl-β-D-ribofuranosyl-imidazole-4-carboxylate³ (0.50 g), 4-nitrobenzylamine hydrochloride (210 mg) and triethylamine (0.16 ml) were stirred in chloroform (30 ml) at room temperature overnight. The solution was washed with saturated sodium bicarbonate solution and water, then evaporated under reduced pressure. The resulting yellow tar was chromatographed on silica gel, eluting with 9:1 methylene chloride:methanol. The collected fractions were monitored by TLC. The like fractions were combined and concentrated under reduced pressure to afford a yellow foam (0.38 g). The foam was dissolved in methanol (20 ml) and methanolic sodium methoxide solution was added (0.3 ml of 0.25M solution). The solution was stirred under an argon atmosphere for 15 min. TLC indicated the reaction was complete. The solution was neutralized to pH 6 with ion exchange resin. The resin was filtered and the solution concentrated under high vacuum to yield a yellow foam (0.23 g). 3 Srivastava, P. C., J. Med. Chem. 17: 1207 (1974).

¹H NMR (DMSO-d₆) Δ ppm, 3.6 (m, 2H, 5′-CH₂) 3.9-4.3 (m, 3H, 2′-CH, 3′-CH, 4′-CH), 4.5 (d, 2H, —CH₂—C₆H₄—NO₂), 5.2-5.4 (br., 3H, 2′-OH, 3′-OH, 5′-OH), 5.5 (d, 1H, 1′-CH), 6.10 (br. s, 2H, 5-NH₂, 7.3 (s, 1H, 2-CH), 7.4-8.2 (AB_{q}, 4H, —C₆H₄—NO₂), 8.3 (t, 1H, 4-CONH).

### Example K

### Preparation of 5-Amino-1-α-D-ribofuranosylimidazole-4-N->(3-chlorophenyl) methyl!carboxamide (Compound No. 24 (1-354))

This compound was prepared according to the procedures described in Example J for the 4-p-nitrobenzyl derivative, substituting 2-chlorobenzylamine for 4-nitrobenzylamine hydrochloride. ¹H NMR (DMSO-d₆) Δ ppm, 3.6 (m, 2H, 5′-CH₂), 3.9-4.3 (m, 3H, 2′-CH, 3′-CH, 4′-CH), 4.4 (d, 2H, —CH₂—O—Cl), 5.1-5.4 (br., 3H, 2′-OH, 3′-OH, 5′-OH), 5.5 (d, 1H, 1′-CH), 6.0 (br.s, 2H, 5-NH₂), 7.2-7.4 (m, 4H, —C₆H₄—Cl), 8.0 (t, 1H, 4-CONH).

### Example L

### Preparation of 5-amino-1-β-D-ribofuranosylimidazole-4-N->(2,4-dichlorophenyl)methyl!carboxamide (Compound No. 25 (1-360))

This compound was prepared according to the procedures described in Example J for the 4-p-nitrobenzyl derivative, substituting 2,4-dichlorobenzylamine for 4-nitrobenzylamine hydrochloride. ¹H NMR (DMSO-d₆), Δ ppm, 3.6 (m, 2H, 5′-CH₂), 3.9-4.3 (m, 3H, 2′-CH, 3′-CH, 4′-CH), 4.4 (d, 2H, —CH₂—C₆H₃—Cl₂), 5.2-5.4 (m, 3H, 2′-OH, 3′-OH, 5′-OH), 5.5 (d, 1H, 1′-CH), 6.0 (br. s, 2H, 5-NH₂), 7.2-7.6 (m, 3H, —C₆H₃—Cl₂), 8.1 (t, 1H, 4-CONH—).

### Example M

### Preparation of 5-amino-2-thio-1-β-D-ribofuranosyl imidazole-4-carboxamide (Compound No. 27 (1-395-0))

To 10 ml of 80% formic acid was added 400 mg of 5-amino-2-thio-1-(2,3-O-isopropylidene-β-D-ribofuranosyl)-imidazole-4-carboxamide.⁴ The resulting mixture was stirred for 1 hour at room temperature. Silica TLC, eluting with 4:1 methylene chloride:methanol, showed conversion of staring material to one major product. The mixture was evaporated to dryness, dissolved in 5 ml of methanol and applied to a 50 ml column of silica gel. The column was eluted with methylene chloride:methanol (5:1). The major product, as determined by TLC, was collected and evaporated to dryness. The residue was dissolved in 3 ml of hot methanol and crystallized upon cooling. Yield was 150 mg of the above-identified product, melting point 205°-208° C. 4 Preparation described in T. Miyoshi, S. Suzaki, A. Yamazaki, Chem. Pharm. Bull., 24 (9): 2089-2093 (1976).

¹H NMR (DMSO-d₆), Δ ppm 3.6 (m, 2H, 5′-CH₂), 3.8 (m, 1H, 4′-CH), 4.1 (m, 1H, 3′-CH), 4.5 (m, 1H, 2′-CH), 5.1 (d, 1H, 2′ or 3′-OH), 5.2 (d, 1H, 2′ or 3′-OH), 5.7 (t, 1H, 5′-OH), 6.3 (d, 1H, 1′-CH), 6.4 (br. s, 2H, 5-NH₂), 6.9 (br. s, 2H, 4-CONH₂), 11.1 (br. s, 1H, 5′-SH).

### Example N

### Preparation of 5-amino-1-(5-chloro-5-deoxy-β-D-ribofuranosyl)imidazole-4-carboxamide (Compound No. 26 (1-332))

AICA riboside (1.00 g), triphenylphosphine (3.05 g) and carbon tetrachloride (1.15 ml) were stirred in dimethyl formamide (38 ml) at room temperature for 3 hours. The solution was diluted with methanol (15 ml), then concentrated under reduced pressure. The resulting yellow tar was chromatographed on silica gel, eluting with 4:1 methylene chloride:methanol. The like fractions were combined and concentrated under reduced pressure to afford a purple foam. The presence of triphenylphosphine oxide, as determined by ¹H NMR, necessitated a second chromotographic step as above. Yield was 0.43 g of a white foam.

¹H NMR (DMSO-d₆), Δ ppm 3.7-3.9 (m, 2H, 5′-CH₂), 4.0-4.4 (m, 3H, 2′-CH, 3′-CH, 4′-CH), 5.4-5.5 (m, 2H, 2′-OH, 3′-OH), 5.6 (d, 1H, 1′-CH), 5.9 (br. s, 2H, 5-NH₂), 6.7-6.9 (br. d, 2H, 4-CONH₂), 7.3 (s, 1H, 2-CH).

### Example 0

### Preparation of 5-amino-1-(2-O-ethyl-β-D-ribofuranosyl)-4-imidazole carboxamide (Compound No. 34 (1-250)) and 5-amino-1-(3-O-ethyl-β-D-ribofuranosyl)-4-imidazole carboxamide (Compound No. 31 (1-251))

A solution of approximately 30 mmol diazoethane in 40 ml of ether was prepared by slow addition of 7 g (44 mmol) of 1-ethyl-3-nitro-1-nitrosoguanidine to a mixture of 8 g of potassium hydroxide, 9 ml water and 60 ml of ether followed by distillation. This was slowly added to a solution of 3.2 g (12 mmol) of 5-amino-1-β-D-ribofuranosylimidazole-4-carboxamide (AICA riboside) in 35 ml dimethylformamide containing 50 mg of tin(II) chloride dihydrate. During the addition approximately 20 ml of methanol was added to maintain solubility. The reaction was filtered to remove a trace precipitate and evaporated to a yellow syrup. Thin layer chromatography on silica gel using methylene chloride/methanol (3:1) showed a major product spot moving faster than AICA riboside. The syrup was chromatographed on silica gel using methylene chloride/methanol (8:1) collecting the major product as determined by TLC. The appropriate fractions were evaporated to a white foam. This was dissolved in 7 ml of methanol. Upon cooling to 4° C. the mixture crystallized to yield 160 mg of 5-amino-1-(2-O-ethyl-β-D-ribofuranosyl)imidazole-4-carboxamide (Compound No. 34 (1-250)) confirmed by NMR decoupling and exchange experiments.

¹H NMR (DMSO-d₆) (for Compound No. 34) Δ ppm, 1.05 (t, 3H, CH₃), 3.3-3.6 (m, 4H, 2′-OCH₂—, 5′-CH₂), 3.9 (m, 1H, 4′-CH), 4.1-4.3 (m, 2H, 2′-CH, 3′-CH), 5.15 (d, 1H, 3-OH), 5.25 (t, 1H, 5′-OH), 5.55 (d, 1H, 1′-CH), 6.0 (br.s, 2H, 5-NH₂), 6.6-6.9 (br.d, 2H, 4-CONH₂), 7.3 (S, 1H, 7-CH).

The supernatant from the above crystallization was cooled overnight at −12° C. yielding a second crop of crystals, 0.58 g, which by NMR decoupling and exchange experiments was shown to be mostly 5-amino-1-(3-O-ethyl-β-D-ribofuranosyl)imidazole-4-carboxamide (Compound No. 31 (1-251)).

¹H NMR (DMSO-d₆) (for Compound No. 31): Δ ppm, 1.1 (t, 3H, CH₃), 3.4-3.7 (m, 4H 3′-OCH₂—, 5′-CH₂), 3.85 (m, 1H, 4′-CH), 4.0 (m, 1H, 3′-CH) 4.4 (q, 1H, 2-CH), 5.25 (t, 1H, 5′-OH), 5.35 (d, 1H, 2′-OH), 5.45 (d, 1H, 1′-CH), 5.9 (br.s, 2H, 5-NH₂), 6.6-6.9 (br.d, 2H, 4-CONH₂), 7.3 (s, 1H, 1-CH). The major impurity was identified as the 2′-O-ethyl isomer.

### Example P

### Preparation of 5-amino-1-(2-O-n-butyl-β-D-ribofuranosyl)imidazole-4-carboxamide and 5-amino-1-(3-O-n-butyl-β-D-ribofuranosyl)imidazole-4-carboxamide (Compound Nos. 32 (1-262) and 33 (1-263))

5-Amino-1-β-D-ribofuranosylimidazole-4-carboxamide (2.50 g, 10.0 mmol) and tin(II) chloride hydrate (35 mg) were dissolved in dimethylformamide (40 ml) and methanol (30 ml). A solution of 0.1 ml of diazobutane⁵ in 150 ml of ether was added in portions. Halfway through the addition, more tin (II) chloride hydrate was added (35 mg). Methanol was added, as needed, to ensure the starting material stayed in solution. The mixture was stirred for 1 hr, then concentrated under reduced pressure to give an oil. Analysis of the oil by ¹H NMR showed mostly N-butylethylcarbamate. The oil was stirred with hexane and decanted to remove the N-butylethylcarbamate. The resulting tar was chromatographed on silica gel using 6:1 methylene chloride:methanol as eluting solvent. The appropriate fractions were combined and concentrated under reduced pressure to give a pink foam. ¹H NMR analysis showed a mixture of 2′ and 3′ butyl ethers. LIPLC analysis showed a 56:28 mixture. The solid was dissolved in isopropanol (2 ml) and cooled. The resulting solid was filtered and dried to give 63 mg. HPLC analysis showed a 77/18 mixture. ¹H NMR decoupling and exchange experiments showed the major product to be the 2′-O-n-butyl ether.

Diazobutane was prepared by treatment of 16.5 g of N-nitroso-N-n-butylmethane>Wilds, A. L. and Meeder, A. L., SOC 13 (1948)! in ethyl ether (100 ml) with potassium hydroxide (55 g) in water (60 ml). The ethereal diazobutane was used without distillation.

¹H NMR (DMSO-d₆) (for Compound No. 32): Δ ppm, 0.8-1.5 (m, 7H, —CH₂ CH₂ CH₃), 3.3-4.2 (m, 7H, 2′-OCH₂—, 2′-CH, 3′-CH, 4′-CH, 5′-CH₂), 5.1 (d, 1H, 3′-OH), 5.3 (t, 1H, 5′-OH), 5.6 (d, 1H, 1′-CH), 6.0 (br.s, 2H, 5-NH₂), 7.6-7.8 (br.d, 2N, 4-CONH₂), 7.3 (s, 1H, 2-CH).

The supernatant from the above crystallization was concentrated under reduced pressure to give 125 mg of a pink foam. HPL analysis showed a 14/71 mixture. ¹H NMR decoupling and exchange experiments showed the major product to be the 3′-O-n-butyl ether.

1H NMR (DMSO-d₆) (for Compound No. 33): Δ ppm, 0.8-1.6 (m, 7H, —CH₂ CH₂ CH₃), 3.4-4.4 (m, 7H, 3′-OCH₂—, 2′-CH, 3′-CH, 4′-CH, 5′-CH₂), 5.2 (t, 1H, 5′-OH), 5.3 (d, 1H, 2′-OH), 5.4 (d, 1H, 1′-CH), 5.9 (br.s, 2H, 5-NH₂), 6.6-6.8 (br.d, 2H, 4-CONH₂), 7.3 (s, 1N, 7-CH).

### Example O

### Preparation of 5-amino-1-β-D-ribofuranosylimidazole-4-N->(3-nitrophenyl)methyl!carboxamide (Compound No. 28 (1-348))

This compound was prepared according to the procedures described in example J for the 4-p-nitrobenzyl derivative, substituting 3-nitrobenzylamine hydrochloride for 4-nitrobenzylamine hydrochloride.

¹H NMR (DMSO-d₆) Δ ppm, 3.6 (m, 2H, 5′-CH₂), 3.9-4.3 (m, 3H, 2′-CH, 3′-CH, 4′-CH), 4.4 (d, 2H, —CH₂—NO₂), 5.2-5.4 (br., 3H, 2′-OH, 3′-OH, 5′-O), 5.5 (d, 1H, 1′-CH), 6.0 (br.s, 2H, 5-NH₂), 7.4 (s, 1H, 7-CH), 7.6-8.2 (m, 4H, —C₆H₄Cl), 8.3 (t, 1H, 4-CONH).

### Example R

### Preparation of 5-amino-1-β-D-ribofuranosylimidazole-4-N->(4-Chlorophenyl)methyl!carboxamide (Compound No. 29 (1-349))

This compound was prepared according to the procedures described in Example J for the 4-p-nitrobenzyl derivative, substituting 4-chlorobenzene amide for 4-nitrobenzylamine hydrochloride.

¹H NMR (DMSO-d₆) Δ ppm, 3.6 (m, 2H, 5′-CH₂), 3.9-4.3 (m, 3H, 2′-CH, 3′-CH, 4′-CH), 4.4 (d, 2H, —CH₂—C₆H₄—Cl), 5.2-5.4 (br., 3H, 2′-OH, 3′-OH, 5′-OH), 515 (d, 1H, 1′-CH) 5.9 (br.s, 2H, 5-NH₂), 7.3-7.4 (m, 5N, —C₆H₄C₁), 7-CH), 8.1 (t, 1H, 4-CONH).

### Example S

### Preparation of 5-amino-1-β-D-ribofuranosylimidazole-4-N->(4-methylphenyl)methyl!carboxamide (Compound No. 30 (1-388))

This compound was prepared according to the procedures described in Example J for the 4-p-nitrobenzyl derivative, substituting 4-methylbenzylamine for 4-nitrobenzylamine hydrochloride.

¹H NMR (DMSO-d₆) Δ ppm, 2.2 (s, 3H, —C₆H₄—CH₃), 3.6 (m, 2H, 5′-CH₂), 3.9-4.3 (m, 5H, 2′-CH, 3′-CH, 4′-CH, —CH₂—C₆H₄—CH₃), 5.2-5.4 (br., 3H, 2′-OH, 3′-OH, 5′-OH), 5.5 (d, 1H, 1′-CH), 5.9 (br.s, 2H, 5-NH₂, 7.1-7.2 (M, 4H, —C₆H₄—CH₃), 7.3 (s, 1H, 7-CH), 7.9 (t, 1H, 4-CONH).

### Example T

### Preparation of 5-amino-1-β-D-ribofuranosyl-imidazole-4-N>(3-chlorophenyl)methyl!carboxamide (Compound No. 35 (1-355))

This compound was prepared according to the procedures described in Example J for the 4-p-nitrobenzyl derivative, substituting 3-chlorobenzylamine for 4-nitrobenzylamine hydrochloride.

¹H NMR (DMSO-d₆) Δ ppm, 3.6 (m, 2H, 5′-CH₂), 3.9-4.3 (m, 3H, 2′-CH, 3′-CH, 4′-CH), 4.3 (d, 2H, —CH₂—C₆H₄—Cl), 5.1-5.4 (br., 3H, 2′-OH, 3′-OH, 5′-OH), 5.5 (d, 1H, 1′-CH), 6.0 (br.s, 2H, 5-NH₂), 7.2-7.4 (m, 4H, —C₆H₄—Cl), 7.4 (s, 1H, 7-CH), 8.1 (t, 1H, 4-CONH).

### Example U

### Preparation of 5-amino-4-(1-piperidinocarbamoyl)-1-β-D-ribofuranosylimidazole (Compound No. 36 (1-207))

This compound in Example J for the 4-p-nitrobenzyl derivative, substituting piperidine for 4-nitrobenzylamine hydrochloride. The product was crystallized from ethanol to give the above-identified product, m.p. 190°-192° C.

sup.¹H NMR (DMSO-d₆) Δ ppm, 1.4-1.7 (M, GH, 3,4,5-CH₂ groups of piperidine ring), 3.55 (m, 2H, 5′-CH₂), 3.8-3.95 (m, 5H, 2- and 6-CH₂ groups of piperidine ring, and 4′-CH), 4.0-4.1 (m, 1H, 3′-CH), 4.25-4.35 (m, 7H, 2-CH) 5.15 (d, 1H, 2′ or 3′-OH), 5.2 (t, 1H, 5′-OH).

### Example V

### Preparation of 5-Amino-1-β-D-ribofuranosyl-imidazole-4-N->p-methoxybenzyl!carboxamide (Compound No. 39 (1-390))

A mixture of the activated succinate ester (0.5 g) (prepared according to Example J), 4-methoxybenzylamine (0:15 ml) and methylene chloride (20 ml) was stirred overnight. TLC indicated completion of the reaction. The solvent was evaporated and the residue was chromatographed over a short silica gel column using a mixture of methylene chloride:methanol (9:1). The fractions containing the product were pooled and evaporated. The residue thus obtained was dissolved in methanol (20 ml) and the pH was adjusted to about 10 by adding a sodium methoxide solution. After stirring the reaction mixture for 45 minutes at room temperature, the solution was neutralized with Dowex 50H+-resin (pH about 6.0). The resin was filtered off, washed with methanol (2×2 ml). The combined filtrate and the washings was evaporated and the residue was crystallized from ethanol. Yield was 100 mg, with a mp of 187°-188° C.

¹H NMR (DMSO-d₆): Δ ppm, 3.55 (m, 2H, 5′-CH₂), 37 (s, 3H, —OCH₃), 3.7-4.1 (m, 3H, 2′-CH, 3′-CH, and 4′-CH), 4.35-4.2 (dd, 2H, —CH₂—N—), 5.1-5.4 (3, m, 3H, 2′-OH, 3′-OH, and 5′-OH), 5.45 (d, 1H, 1-CH), 5.9 (br. 2H, NH₂), 6.8-7.2 (m, 4H, aromatic-phenyl), 7.3 (s, 17H, C₂—H), and 7.85 (t, 1H, C—NH).

### Example W

### Preparation of 5-Amino-1-β-D-ribofuranosylimidazole-4-N(4-dimethylaminobenzyl)-carboxamide hydrochloride (Compound No. 41 (1-396-3))

To a suspension of 4-dimethylaminobenzylamine hydrochloride (245 mg, 2 mmol) in methylene chloride (25 ml), triethylamine (222 mg, 2 mmol) was added and the resulting mixture stirred 45 minutes to it was added the activated succinate ester prepared according to example J (500 mg); the resulting mixture was stirred at room temperature overnight. TLC indicated completion of the reaction. The reaction mixture was evaporated and the residue was chromatographed through a short silica gel column using a mixture of methylene chloride-methanol (9:1). Fractions showing the major product were pooled and evaporated to dryness. The residue was dissolved in methanol (15 ml) and the pH was adjusted to about 10 using a sodium methoxide solution. After stirring at room temperature for 45 minutes, the solution was neutralized with Dowex 50-resin. The resin was filtered off and washed with methanol (2×5 ml). The combined filtrate and the washings were evaporated to dryness. The residue which was in the form of a foam was dissolved in absolute ethanol (10 ml). The pH of the solution was adjusted to about 5 with an ethanolic-HCl solution. Solvent was evaporated to dryness and the residue was treated with anhydrous ether. The amorphous solid that separated was collected by filtration and washed with ether (2×10 ml), and dried under high vacuum to yield 250 mg. The compound obtained was highly hygroscopic; no melting point could be obtained.

sup.¹H NMR (D₂O) Δ ppm, 3.05 (s, 6H, N(CH₃)₂), 3.6 (m, 2H, 5′-CH), 3.8-4.3 (3m, 3H, 2′-CH, 3′-CH, and 4′-CH), 4.4 (s, 2H, CH₂—N—), 5.5 (d, 1H, 1′-CH), 7.3-7.4 (m, 4H, phenyl), and 7.9 (s, 1H, 2-CH).

### Example X

### Preparation of (R)-5-Amino-1-β-D-ribofuranosylimidazole-4-N->2-hydroxy-2-(3,4-dihydroxyphenyl)ethyl!carboxamide (Compound 42 (1-431))

This compound was prepared according to the procedure described in Example J substituting (R)-norepinephrine for 4-nitrobenzylamine hydrochloride and dimethylformamide in place of chloroform as the reaction solvent.

¹H NMR (DMSO-d₆): Δ ppm, 3.1-3.3 (m, 2H, —CH₂—N), 3.5-3.6 (m, 2H, 5-CH₂), 3.8-3.9 (m, 1H, 4′-CH) 4.0-4.1 (m, 1H, 3′-CH) 4.2-4.3 (m, 1H, 2′-CH), 4.4-4.5 (m, 1H, phenyl-CH—OH), 5.2-5.2 (m, 1H, 2′ or 3′-OH), 5.2-5.3 (t, 1H, 5′-OH) 5.3-5.4 (m, 1H, 2′ or 3′-OH), 5.4-5.5 (d, 1H, 1′-CH), 5.9 (br. s, 2H, 5-NH₂), 6.5-6.8 (m, 3H, aryl of catechol), 7.1 (t, 1H, 4-CONH), 7.3 (s, 1H, 2-CH), 7.2-7.8 (br. s, 2H, catechol-OH).

### Example Y

### Preparation of 5-Amino-2-thiophenyl-1-β-D-ribofuranosylimidazole-4-carboxamide (Compound No. 43 (1-432))

5-Amino-2-bromo-1-(2,3-O-isopropylidene-β-D-ribofuranosyl)imidazole-4-carboxamidel (1.1 g), thiophenol (1.3 g) and triethylamine (0.61 g) were refluxed in a mixture of 25 ml methanol and 3 ml of 1N sodium hydroxide for 18 hours. The reaction mixture was concentrated and the residue mixed with 40 ml of methylene chloride. The methylene chloride mixture was washed with water and saturated sodium bicarbonate and dried over magnesium sulfate. The methylene chloride was evaporated and the residue purified by chromatography on 200 ml of silica gel using a mixture of methylene chloride and methanol (95:5), yielding 0.5 g of 5-amino-2-thiophenyl-1-(2,3-O-isopropylidene-β-D-ribofuranosyl)imidazole-4-carboxamide. Treatment of that compound with 80% formic acid for 3 hours at room temperature to remove the isopropylidene group followed by evaporation and purification by silica chromatography using methylene chloride:methanol (9:1) yielded 250 mg of the title compound as a white foam.

¹ Miyosi T., Chem. Pharm. Bull. 24: 2089 (1976).

¹H NMR (DMSO-d₆) Δ ppm, 3.3-3.5 (m, 2H, 5′-CH₂), 3.8-3.9 (m, 1H, 4′-CH) 4.0-4.1 (m, 1H, 3′-CH), 4.5 (q, 1H, 2′-CH) 5.1 (d, 1H, 2′- or 3′-OH), 5.3 (d, 1H, 2′- or 3′-OH), 5.7 (t, 1H, 5′-OH), 5.9 (d, 1H, 1′-CH) 7.5 (br. s, 2H, 4-NH₂), 6.7 and 7.1 (br s, 2H, CONH₂) 7.1-7.5 (m, 5H, phenyl).

### Example Z

### Preparation of 5-Amino-1-β-D-ribofuranosylimidazole-4-N-(2-endo-norbornyl)carboxamide) (Compound No. 45 (1-438))

A mixture of (±) endo-2-aminonorbornane hydrochloride (240 mg), triethylamine (160 mg) and methylene chloride was stirred at room temperature for 45 minutes under argon. To it was added activated succinate ester (See Example J) (750 mg) and stirred overnight. TLC indicated completion of the reaction. Solvent was evaporated and the residue chromatographed over silica gel column using a mixture of methylene chloride methanol (9:1). Fractions containing the product were pooled and evaporated. The residue was dissolved in methanol (25 ml) and the pH was adjusted to about 10 with a sodium methoxide solution. After stirring for 45 minutes at room temperature the solution was neutralized with H+ resin (pH approximately 6). The resin was filtered off and washed with methanol. The combined washings and the filtrate was evaporated and the residue kept under high vacuum to obtain a solid glossy product. Yield was 280 mg.

¹H NMR (DMSO-d₆) Δ ppm, 1.1-2.4 (m, 10H, norbonyl), 3.6 (br.M, 2H, 5′-CH₂), 3.9 (m, 1H, —N—CH), 4-4.4 (2 m, 3H, 2′-CH, 3′-CH and 4′-CH), 5.05, and 5.35 (2-d, 2H, 2′-OH and 3′-OH), 5.25 (t, 1H, 5′-OH), 5.5 (d, 1H, 1′-CH), 5.9 (br. 2H, NH₂) 6.8 (d, 1H, —NH—CO), 7.25 (S, 1H, 2-CH).

### Example AA

### Preparation of 5-Amino-1-β-D-ribofuranosyl-imidazole-4-N->(3-iodophenyl)methyl!carboxamide (Compound No. 44 (1-434))

This compound was prepared according to the procedures described in Example J for the 4-p-nitrobenzyl derivative, substituting 3-iodobenzylamine hydrochloride for 4-nitrobenzylamine hydrochloride.

¹H NMR (DMSO-d₆) Δ ppm, 3.6 (m, 2H, 5′-CH₂), 3.9-4.3 (m, 3H, 2′-CH, 3′-CH, 4′-CH), 4.3 (d, 2H, —CH₂—C₆H₄—I), 5.2-5.4 (m, 3H, 2′-OH, 3′-OH, 5′-OH), 5.5 (d, 1H, 1′-CH), 5.9 (br.s, 2H, 5-NH₂), 7.1-7.7 (m, 4H, —C₆H₄), 7.3 (s, 1H, 2-CH), 8.1 (t, 1H, 4-CONH—)

### Example AB

### Preparation of 5-Amino-1-(5-iodo-5-deoxy-β-D-ribofuranosyl)imidazole-4-N->(4-nitrophenyl)methyl!carboxamide (Compound No. 46 (1-44

The compound used in this procedure, 5-amino-1-(5-iodo-5-deoxy-2,3-isopropylidene-β-D-ribofuranosyl)imidazole-4-N->(4-nitrophenyl)methyl !carboxamide, was prepared by the same reaction sequence (stopping at step B) described in Example AH for compound 53 (1-468), substituting the 4-N-p-nitrobenzylamide (compound 23 (1-343)) for the 4-N-p-chlorobenzylamide (compound 29 (1-349)).

5-Amino-1-(5-iodo-5-deoxy-2,3-O-isopropylidene-β-D-ribofuranosyl)imidazole-4-N->(4-nitrophenyl)methyl!carboxamide (200 mg) was dissolved in 10 ml of 80% formic acid. The solution was stirred at 45° C. for 2 hours. The solvents were evaporated under reduced pressure and the resulting residue co-evaporated twice with water and twice with methanol. The residue was chromatographed on silica gel, using 6/1 methylene chloride/methanol as eluting solvent. The appropriate fractions were combined and concentrated under reduced pressure to yield 60 mg of the above-identified compound as a yellow foam.

¹H NMR (DMSO-d₆) Δ ppm, 3.3-3.6 (m, 2H, 5′-CH₂), 3.8-4.4 (m, 3H, 2′-CH, 3′-CH₄′-CH), 4.5 (d, 2H, CH₂—C₆H₄NO₂), 5.4-5.5 (m, 2H, 2′-OH, 3′-OH), 5.6 (d, 2H, 1′-CH), 5.9 (br.s, 2H, 5-NH₂), 7.4 (S, 1H, 2-CH), 7.5-8.2 (m, 4H, C₆H₄—NO₂, 8.3 (4,1H, 4-CONH—).

### Example AC

### Preparation of 5-Amino-1-β-D-ribofuranosylimidazole-4-carboxylic Acid, p-Nitrobenzylthio Ester (Compound No. 47 (1-450))

5-Amino-1(2,3,5-tri-O-acetyl-β-D-ribofuranosyl)imidazole-4-carboxylic acid¹ (1.0 g) was dissolved in 8 ml of thionyl chloride under argon with stirring for 10 minutes. The mixture was evaporated under vacuum and the residue was dissolved in 15 ml of tetrahydrofuran containing 2.0 g of p-nitrobenzyl mercaptan. Triethylamine (1.5 ml) was added and the mixture stirred under argon for 20 minutes. The reaction is evaporated to a gum and the residue mixed with 50 ml of methylene chloride and washed with 2×25 ml of water. The methylene chloride phase was dried over magnesium sulfate and evaporated to a syrup which was purified by chromatography on silica gel using a mixture of ethyl acetate and methylene chloride (1:1) yielding 500 mg of 5-amino-1-(2,3,5-tri-O-acetyl-β-D-ribofuranosyl)imidazole-4-carboxylic acid, p-nitrobenzylthio ester. Treatment with sodium methoxide in 30 ml of dry methanol such that a slightly basic pH was maintained until deacetylation was complete (as determined by thin layer chromatography), followed by neutralization with Dowex 50 (H+) and evaporation yielded the desired compound contaminated with a product presumed to be the methyl ester. Purification by chromatography on silica using a mixture of methylene chloride and methanol (9:1) gave 38 mg of the desired compound as a yellow foam.

¹Srivastava, P. C., J. Med. Chem. 17: 1207 (1974).

¹H NMR (DMSO-d) Δ ppm, 3.5-3.7 (m, 2H, 5′-CH₂), 3.9-4.0 (m, 1H, 4′-CH), 4.2-4.4 (m, 2H, 2′- and 3′-CH), 5.2 (d, 1H, 2′- or 3′-OH), 5.3-5.5 (m, 2H, 5′ and 2′- or 3′-OH), 5.6 (d, 1H, 1′-CH), 6.9 (br. s, 2H, 5-NH₂), 7.4 (s, 1 h, 2-CH), 7.6 and 8.2 (d, 2H, phenyl).

### Example AD

### Preparation of 5-Amino-1-β-D-ribofuranosyl-imidazole-4-N-indolinylcarboxamide (Compound No. 48 (1-452))

This compound was prepared according to the procedures described in Example J for the 4-p-nitrobenzyl derivative, substituting indoline for 4-nitrobenzylamine hydrochloride.

¹H NMR (DMSO-d₆) Δ ppm, 3.1 (t, 2H, indolinyl-CH₂), 3.6 (m, 2H, 5′-CH₂—), 5.2-5.4 (m, 3H, 2′-OH, 3′-OH, 5′-OH), 5.5 (d, 1H, 1′-CH), 6.4 (br.s, 2H, 5-NH₂), 6.9-8.1 (m, 4H, indolinyl aromatics), 7.4 (S, 1H, 2-CH).

### Example AE

### Preparation of (R)-5-Amino-1-β-D-ribofuranosylimidazole 4-N->1-4-nitrophenyl)ethyl!carboaxamide (Compound No. 49 (1-453))

This compound was prepared according to the procedures described in Example J for the 4-p-nitrobenzyl derivative, substituting (R)-4-nitro-α-methylbenzylamine hydrochloride for 4-nitrobenzylamine hydrochloride.

¹H NMR (DMSO-d₆) Δ ppm, 1.5 (d, 3H, α-methyl on N⁴-benzyl carboxamide), 3.6 (m, 2H, 5′-CH₂), 3.9-4.3 (m, 3H, 2′-CH, 3′-CH, 4′-CH), 5.1 (m, 1H, methine proton on N4-benzylcarboxamide), 5.1-5.4 (m, 3H, 2′-OH 3′-OH, 5′-OH), 5.5 (d, 1H, 1′-CH), 7.3 (s, 1H, 2-CH), 7.6-8.2 (m, 4H, C₆H₄—NO₂), 8.0 (d, 1H, 4-CONH—).

### Example AF

### Preparation of (S)-5-Amino-1-β-D-ribofuranosylimidazole-4-N->(4-nitrophenyl)ethyl!carboxamide (Compound No. 50 (1-459))

This compound was prepared according to the procedures described in Example) for the 4-p-nitrobenzyl derivative, substituting (S)-4-nitro-α-methylbenzylamine hydrochloride for 4-nitrobenzylamine hydrochloride.

¹H NMR (DMSO-d₆) Δ ppm, 1.5 (d, 3H, α-methyl on N⁴-benzyl carboxamide), 3.6 (m, 2H, 5-CH₂), 3.9-4.3 (m, 3H, 2′-CH, 3′-CH, 4′-CH), 5.1 (m, 1H, methine proton on N4-benzylcarboxamide), 5.1-5.4 (m, 3H, 2′-OH, 3′-OH, 5′-OH), 5.5 (d, 1H, 1′-CH′ 5.9 (br.s, 2H, 5-NH₂), 7.4 (s, 1H, 2-CH), 7.6-8.2 (m, 4H, C₆H₄NO₂) 8.0 (d, 1H, 4-CONH—).

### Example AG

### Preparation of 5-Amino-1-(5-chloro-5-deoxy-β-D-ribofuranosyl)imidazole-4-N->4-nitrophenyl)methylethyl!carboxamide (Compound No. 51 (1-466))

5-amino-1-β-D-ribofuranosylimidazole-N->(4-nitrophenyl)methyl!carboxamide, Compound 23 (1-343) (0.5 g), triphenylphosphine (1.00 g), carbon tetrachloride (0.37 ml), and THF (25 ml) were combined and stirred at ambient temperature, under argon, overnight. A white precipitate formed. Dimethylformamide (8 ml) was added and the solution was stirred at ambient temperature, under argon, overnight. The solvent was evaporated under reduced pressure and the resulting oil co-evaporated with methanol (3×20 ml). The resulting viscous oil was chromatographed on silica gel, using 7:1 methylene chloride:methanol as eluting solvent. The appropriate fractions were combined and concentrated in vacuo to give a yellow foam (0.28 g). The foam was crystallized from cold methanol to give yellow crystals (200 mg), mp=174°-176° C.

¹H NMR (DMSO-d₆) Δ ppm 3.7-3.9 (m, 2H, 5′-CH₂), 4.0-4.4 (m, 3H, 2′-CH, 3′-CH, 4′-CH), 4.5 (d, 2H, —CH₂—C₆H₄NO₂), 5.4-5.6 (m, 2H, 2′-OH, 3′-OH), 5.6 (d, 1H, 1′-CH), 5.9 (br.s, 2H, 5-NH₂), 7.4 (s, 1H, 2-CH), 7.5-8.2 (m, 4H, —C₆H₁₄NO₂), 8.3 (t, 1H, 4-CONH—).

### Example AH

### Preparation of 5-Amino-1-(5-azido-5-deoxy-β-D-ribofuranosyl)imidazole-4-N->(4-chloro phenyl)methyl!carboxamide (compound 52 (1-467)) and 5-Amino-1-(5-amino-5-deoxy-β-D-ribofuranosyl)imidazole-4-N->(4-chloro phenyl)methyl!carboxamide Hydrochloride (Compound No. 53 (1-468))

### A. Preparation of 5-Amino-1-(2,3-O-isopropylidene-β-D-ribofuranosyl)imidazole-4-N->(4-chlorophenyl)methyl!carboxamide

Compound 29 (1-349), (6.8 g, 17.8 mmole), was dissolved in a mixture of 100 ml DMF, 15 ml acetone and 15 ml 2,2-dimethoxypropane. Hydrogen chloride gas (approximately 1.0 g) was added and the mixture stirred under argon for 4 hours. The mixture was poured into 50 ml of saturated sodium bicarbonate and evaporated under vacuum at 45° C. The residue dissolved in a mixture of 100 ml ethyl acetate and 25 ml water. The ethyl acetate phase was separated and washed with 25 ml of water, dried over magnesium sulfate and concentrated to a foam. TLC (silica gel, 9:1 methylene chloride:methanol) showed a significant faster moving impurity in the product which was identified as the 5′-(2-methoxypropane) mixed ketal of the above-identified compound. This was converted to the above-identified compound by dissolving the foam in 100 ml of methanol and adjusting the pH to 2.5 with ethanolic hydrogen chloride. After 30 minutes the mixture was neutralized with saturated sodium bicarbonate and concentrated to a slurry. This was dissolved in 100 ml of methylene chloride, washed with 25 ml of water. The methylene chloride phase was dried over magnesium sulfate and concentrated to a foam. Drying under vacuum at 40° C. for 18 hours yielded 7.2 g (96%) of the above-identified compound.

### B. Preparation of 5-Amino-1-(5-iodo-5-deoxy-2,3-isopropylidene-β-D-ribofuranosyl)imidazole-4-N->(4-chlorophenyl)methyl!carboxamide

A mixture of the product of Step A (25 g, 59 mmole) and methyltriphenoxyphosphonium iodide (76 g, 166 mmole) in 500 ml of methylene chloride was stirred for 30 minutes at room temperature under argon. The resulting solution was extracted with 150 ml of water, 150 ml of 5% sodium thiosulfate, 150 ml of 1N sodium hydroxide, 100 ml of water and dried over magnesium sulfate. The solvent was removed under vacuum and the resulting oil applied to a 1.31 column of flash grade silica gel prepared in 2:1 hexane:ethyl actetate. The column was eluted with the same solvent to remove impurities then 1:1 hexane:ethyl acetate was used to elute the desired product. Appropriate fractions were combined and evaporated to yield 24.4 g of the above-identified compound as a gummy solid. Impure fractions were again subjected to chromatography to yield an additional 2.3 g of the above-identified product. Total yield was 26.7 g (85%).

### C. Preparation of 5-amino-1-(5-azido-5-deoxy-2,3-O-isopropylidene-β-D-ribofuranosyl)imidazole-4-N->(4-chlorophenyl)methyl!carboxamide

A mixture of the product of Step B (26.7 g, 50 mmole), lithium azide (14 g, 285 mmole) and 100 mg of 18-crown-6 in 350 ml of DMF was stirred for 8 hours at room temperature under argon. The slurry was concentrated to remove solvent and the residue dissolved in a mixture of 500 ml of ethyl acetate and 100 ml of water. The ethyl acetate phase was separated, washed with water and saturated sodium chloride, and then dried over magnesium sulfate. Evaporation of the solvent yielded 25 g of the above-identified compound as a yellow gum which still contained solvent. This was used in the next step without further purification.

### D. Preparation of 5-Amino-1-(5-azido-5-deoxy-β-D-ribofuranosyl)imidazole-4-N->(4-chloro phenyl)methyl!carboxamide. (Compound No. 52 (1-467))

The product of Step C, as obtained, was dissolved in 150 ml of 80% trifluoracetic acid and warmed to 50° C. for 30 minutes. The solution was evaporated to a syrup at 40° C. under vacuum and the residue evaporated twice from 25 ml of water. The syrupy residue was dissolved in 100 ml of ethyl acetate and gently stirred over 100 ml of saturated sodium bicarbonate. Crystallization began in the ethyl acetate phase and after 1 hour crystals were collected by filtration. These crystals were combined with two additional crops or crystals obtained by concentration of the ethyl acetate phase to yield 15.7 g (77% yield based on the product of Step B). Melting point of an analytical sample was 182°-183° C.

¹H NMR (DMSO-d₆) Δ ppm, 3.6 (M, 2H, 5′-CH₂), 4.0-4.3 (m, 3H, 2′-CH, 3′-CH, 4′-CH), 4.3 (d, 2H, —CH₂ C₆H₄C₁), 5.4-5.5 (m, 2H, 2′-OH, 3′-OH), 5.5 (d, 1H, 1′-CH), 5.9 (br.s, 2H, 5-NH₂), 7.3-7.4 (m, 4H, C₆H₄Cl), 7.4 (s, 1H, 2-CH), 8.1 (t, 1H, 4-CONH—). IR (KBr) cm⁻¹, 2110.

### E. Preparation of 5-Amino-1-(5-amino-5-deoxy-β-D-ribofuranosyl)imidazole-4-N->(4-chloro phenyl)methyl carboxamide

Compound 52 (1-467) (6.5 g, 159 mmole) was dissolved in 500 ml of boiling ethanol. After cooling to 40° C. the solution was saturated with argon and 0.5 g of 10% palladium on carbon added. The mixture was stirred under a hydrogen atmosphere for 8 hours. The mixture was saturated with argon and filtered through Celite 505 and concentrated to a syrup which was used in the next step without further purification.

### F. Preparation of 5-Amino-1-(5-amino-5-deoxy-β-D-ribofuranosyl)imidazole-4-N->(4-chloro phenyl)methyl!carboxamide Hydrochloride (Compound No. 53 (1-468))

The product of Step E (theoretically 159 mmole) was dissolved in 100 ml of ethanol and 3.5 ml of 6N hydrochloric acid added (pH to wet pH paper approximately 3). The solution was evaporated to a hard syrup. This syrup was dissolved in 50 ml of hot ethanol and diluted with 150 ml of ethyl ether. The resulting gummy precipitate was stirred sealed for 12 hours and the resulting white precipitate collected by filtration and washed with ether. Drying under vacuum at 40° C. yielded 6.0 g of the above-identified compound (90% yield based on the compound from Step D).

¹H NMR (DMSO d₆) Δ ppm, 3.0-3.2 (m, 2H, 5′-CH₂), 4.0-4.4 (M, 3H, 2′-CH, 3′-CH, 4′-CH), 4.4 (d, 2H, —CH₂—C₆H₄Cl), 5.8-6.2 (br., 2H, 2′-OH, 3′-OH), 7.2-7.4 (m, 4H, C₆H₄Cl), 7.8 (s, 1H, 2-CH), 8.3 (br., 3H, NH₂.HCl).

### Example AI

### Preparation of 5-Amino-1-(5-amino-5-deoxy-β-D-ribofuranosyl)imidazole-4-N-(cyclopentyl)carboxamide Hydrochloride ((Compound No. 37) 1-270))

This compound was prepared by the same reaction sequence described in Example AH for compound 53 (1-468), substituting the 4-N-cyclopentylamide, compound 10 (1-186), of Table XII for the 4-N-p-chlorobenzylamide compound 29 (1-349) of Table XII.

¹H NMR (DMSO-d₆) Δ ppm, 1.4-1.9 (m, 9H, cyclopentyl aliphatic protons), 3.0-3.2 (m, 2H, 5′-CH₂), 4.0-4.3 (m, 3H, 2′-CH, 3′-CH, 4′-CH), 5.5 (d, 1H, 1′-CH), 5.9 (br.s, 2H, 5-NH₂), 7.1 (d, 1H, 4-CONH—), 7.4 (s, 1H, 2-CH).

### Example AJ

### Preparation of 5-Amino-1-(5-deoxy-5-methylthio-β-D-ribofuranosyl)imidazole-4-carboxamide (Compound No. 54 (1-483))

The intermediate, 5-amino-1-(5-chloro-5-deoxy-β-D-ribofuranosyl)imidazole-4-carboxamide, was prepared according to the procedures described in Example AI for compound 5¹ (1-466), substituting 5-amino-1-β-D-ribofuranosylimidazole-4-carboxamide for 5-amino-1-β-D-ribofuranosylimidazole-4-N->(4-nitrophenylmethyl!carboxamide.

To a 0.1N sodium methoxide/methanol solution, at 0° under argon, was bubbled methyl mercaptan. To the resulting 0.1 N sodium methylthiolate/methanol solution was added 5-amino-1-(5-chloro-5-deoxy-β-D-ribofuranosyl)imidazole-4-carboxamide (0.40 g). The solution was heated of reflux overnight. The solution was cooled and neutralized with Dowex 50 strongly acidic ion exchange resin. The mixture was filtered and concentrated under reduced pressure. The resulting residue was chromatographed on silica gel, using 4:1 methylene chloride:methanol as the eluting solvent. The appropriate fractions were combined, concentrated under reduced pressure, and vacuum dried to give the above-identified compound as a white foam (0.28 g).

¹H NMR (DMSO-d₆) Δ ppm, 2.1 (s, 3H, —S—CH₃), 3.7-3.9 (m, 2H, 5′-CH₂), 3.9-4.4 (m, 3H, 2′-CH, 3′-CH, 4′-CH), 5.3-5.4 (m, 2H, 2′-OH, 3′-OH), 5.5 (d, 1H, 1′-CH), 5.8 (br.s, 2H, 5-NH₂), 6.6-6.9 (br.m, 2H, 4-CONH₂), 7.3 (s, 1H, 2-CH).

### Example AK

### Preparation 5-Amino-1-β-D-ribofuranosylimidazole-4-N-(4-bromophenyl)carboxamide (Compound No. 55 (1-484))

5-Amino-1-(2,3,5-tri-O-acetyl-β-D-ribofuranosyl)imidazole-4-carboxylic acid (Srivastava, P. C., et al., J. Med. Chem. 17 1207, (1974), (0.75 g) and thionyl chloride (7 ml) were stirred at ambient temperature under a drying tube, for 15 minutes. The excess thionyl chloride was evaporated under reduced pressure and the resulting residue co-evaporated with methylene chloride (3×20 ml). The resulting yellow foam was dissolved in methylene chloride (40 ml) and 4-bromoaniline (0.35 g) was added. Triethylamine (approximately 0.75 ml) was added until the solution was basic. The solution was stirred at ambient temperature under a drying tube for 2 hours. The solution was washed with water, dried with magnesium sulfate, and concentrated under reduced pressure to give a yellow foam. The foam was dissolved in methanol (35 ml). A sodium methoxide methanol solution (approximately 0.75 ml of a 0.5N solution) was added and the resulting solution stirred at ambient temperature under a drying tube, for 30 minutes. The solution was neutralized with methanol-washed Dowex 50 (strongly acidic ion-exchange resin). The mixture was filtered and concentrated under reduced pressure to give a pale yellow residue. The residue was crystallized from methanol (15 ml)/methylene chloride (10 ml) to give tan crystals (0.23 g). The crystals were recrystallized to give off-white crystals (90 mg). Mp: 214°-216° C. (decomp).

¹H NMR (DMSO-d₆) Δ ppm, 3.6 (m, 2H, 5′-CH₂), 3.9-4.3 (m, 3H, 1′-CH, 3′-CH, 4′-CH), 5.2-5.4 (m, 3H, 2′-OH, 3′-OH, 5′-OH), 5.5 (d, 1H, 1′-CH), 6.2 (br.s, 2H, 5-NH₂), 7.4-7.8 (m, 4H, —C₆H₄Br), 7.4 (s, 1H, 2-CH), 9.5 (s, 7H, 4-CONH).

### Example AL

### Preparation of 5-Amino-1-β-D-ribofuranosyl-imidazole-4-N->(4-bromophenyl)methyl!carboxamide (Compound No. 56 (1-487))

This compound was prepared according to the procedures described in Example J for the 4-p-nitrobenzyl derivative, substituting 4-bromobenzylamine hydrochloride for 4-nitrobenzylamine hydrochloride.

¹H NMR (DMSO-d₆) Δ ppm, 3.5-3.6 (m, 2H, 5′-CH₂), 3.9-4.3 (m, 3H, 2′-CH, 3′-CH, 4′-CH), 4.3 (d, 2H, CH₂—C₆H₄Br), 5.1-5.4 (m, 3H, 2′-OH, 3′-OH, 5′-OH), 5.5 (d, 1H, 1′-CH), 5.9 (br.s, 2H, 5-NH₂), 7.2-7.5 (m, 4H, —C₆H₄Br), 7.3 (s, 4H, 2-CH), 8.0 (t, 1H, 4-CONH—).

### Example AM

### Preparation of 5-Amino-1-β-D-ribofuranosyl-imidazole-4-N-(4-iodophenyl) carboxamide (Compound No. 57 (1-488))

This compound was prepared according to the procedures described in Example AK for the 4-p-bromophenyl derivative, substituting 4-iodoaniline for 4-bromoaniline. The final product was recrystallized from ethanol. Mp: 227°-229° C. H NMR (DMSO-d₆) Δ ppm, 3.5-3.6 (m, 2H, 5′-CH₂), 3.9-4.4 (m, 3H, 2′-CH, 3′-CH, 4′-CH), 5.2-5.4 (m, 3H, 2′-OH, 3′-OH, 5′-OH), 5.5 (d, 1H, 1′-CH), 6.2 (br.s, 2H, 5-NH₂), 7.4 (s, 1H, 2-CH), 7.6-7.7 (m, 4H, —C₆H₄ I), 9.5 (s, 1H, 4-CONH).

### Example AN

### Preparation of 5-Amino-1-β-D-ribofuranosylimidazole-4-N-(4-nitrophenyl)carboxamide (Compound No. 58 (1-489))

This compound was prepared according to the procedures described in Example AK for the 4-p-bromophenyl derivative, substituting 4-nitroaniline for 4-bromoaniline. The final product was recrystallized from methanol to give a yellow powder.

¹H NMR (DMSO-d) Δ ppm, 3.5-3.6 (m, 2H, 5′-CH₂), 3.9-4.4 (m, 3H, 2′-CH, 3′-CH, 4′-CH), 5.2-5.4 (m, 3H, 2′-OH, 3′-OH, 5′-OH), 5.6 (d, 1H, 1′-CH), 6.4 (br.s, 2H, 5-NH₂), 7.5 (s, 1H, 2-CH), 8.1-8.3 (m, 4H, C₆H₄NO₂), 10.1 (s, 1H, 4-CONH).

### Example AO

### Preparation of 5-Amino-1-β-D-ribofuranosyl-imidazole-4-N->2-(4-nitrophenyl)ethyl carboxamide (Compound No. 59 (1-506))

This compound was prepared according to the procedures described in Example J for the 4-p-nitrobenzyl derivative, substituting 4-nitrophenethylamine hydrochloride for 4-nitrobenzylamine hydrochloride.

¹H NMR (DMSO-d) Δ ppm, 2.9-3.0 (t, 2H, —CH₂—C₂H₄—NO₂), 3.4-3.6 (m, 2H, 5′-CH₂), 3.9-4.3 (m, 3H, 2′-CH, 3′-CH, 4′-CH), 4.8-5.4 (br., 3H, 2′-OH, 3′-OH, 5′-OH), 5.5 (d, 1H, 1′-CH), 5.9-6.2 (br., 2H, 5-NH₂), 7.5-8.2 (m, 4H, —C₆H₄NO₂), 7.6 (s, 1H, 2-CH), 7.7 (t, 1H, 4-CONH).

### Example AP

### Preparation of 5-Amino-4->1->4-(4-nitrophenyl)!piperazinocarbamoyl!-1-β-D-ribofuranosylimidazole (Compound No. 60 (1-508))

This compound was prepared according to the procedures described in Example J for the 4-nitrobenzyl derivative, but substituting 1-(4-nitrophenyl)piperazine for 4-nitrobenzylamine hydrochloride. The product as recrystallized from cold methanol and had a mp of 199°-200° C.

¹H NMR (DMSO-d) Δ ppm, 3.4-3.6 (m, 10H, 3′-CH₂, piperazonyl methylenes), 3.9-4.3 (m, 3H, 2′-CH, 3′-CH, 4′-CH), 5.2-5.4 (m, 3H, 2′-OH, 3′-OH, 5′-OH), 5.5 (d, 1H, 1′-CH), 6.3 (br.s, 2H, 5-NH₂), 7.0-8.1 (m, 4H, —C₆H₄NO₂), 7.3 (s, 1H, 2-CH).

### Example AQ

### Preparation of 5-Amino-1-(5-deoxy-β-D-ribofuranosyl)imidazole-4N->(4-chlorophenyl)methyl!carboxamide (Compound No. 61 (1-509))

5-Amino-1-(5-iodo-5-deoxy-2,3-isopropylidene-β-D-ribofuranosyl)imidazole-4-N->(4-chlorophenyl)methyl!carboxamide (see procedures described in Example AH for preparation of Compound 53 (1-468), step B) (0.64 g) was stirred in 30 ml of 50% formic acid overnight. The excess solvent was evaporated under reduced pressure. The resulting residue was co-evaporated with water (25 ml) and methanol (25 ml). The resulting yellow foam was chromatographed on silica gel, using 9:1 methylene chloride:methanol as eluting solvent. The appropriate fractions were combined and concentrated under reduced pressure to give 0.47 g of 5-amino-1-(5-iodo-5-deoxy-β-D-ribofuranosyl)imidazole-4-N->(4-chlorophenyl)methyl!carboxamide.

5-Amino-1-(5-iodo-5-deoxy-β-O-ribofuranosyl)imidazole-4-N->(4-chlorophenyl)methyl!carboxamide (0.04 g), palladium on carbon 10% (20 mg), and ethanol (20 ml) were charged to a Parr bottle. The bottle and contents were charged with 45 p.s.i. hydrogen. The reaction progress was monitored by HPLC (Waters C18, 55% methanol/45% 0.1N acetic acid, 260 nm, 1.0 ml/min). After 24 hour, there was 34% starting material. Fresh catalyst was added (20 mg) and the mixture re-charged with hydrogen (45 p.s.i.). The mixture was shaken for an additional 48 hours. The reaction mixture contained 30% starting material. The mixture was filtered through Celite, and concentrated under reduced pressure. The resulting residue was chromatographed on silica gel, using ethyl acetate (400 ml) and 5% methanol in ethyl acetate (200 ml) as the eluting solvent. The appropriate fractions were combined and concentrated under reduced pressure to yield 70 mg of a white foam. HPLC indicated 9% starting material. The material was rechromatographed on silica gel, using ethyl acetate as eluting solvent. All fractions containing less than 3% starting material were combined and concentrated under reduced pressure to yield 36 mg of the above-identified compound as a pink foam.

¹H NMR (DMSO-d) Δ ppm, 1.2-1.3 (d, 3H, 5′-CH₃), 3.7-4.3 (m, 3H, 2′-CH, 3′-CH₂ 4′-CH), 4.3 (d, 2H, CH₂—C₆H₄Cl), 5.1-5.4 (m, 3H, 2′-OH, 3′-OH, 1′-CH), 5.8 (br.s, 2H, 5-NH₂), 7.2-7.4 (m, 5H, C₆H₄Cl, 2-CH), 8.1 (t, 1H, 4-CONH).

### Example AR

### Preparation of 5-Amino-1-(5-deoxy-5-methylsulfinyl-β-D-ribofuranosyl)imidazole-4-carboxyamide (Compound No. 62 (1-510))

5-Amino-1-(5-deoxy-5-methylthio-β-D-ribofuranosyl)imidazole-4-carboxamide (compound 54 (1-483)) of Example AK (0.40 g) was dissolved in water (20 ml). Hydrogen peroxide, 30 weight percent, (0.42 ml), was added and the solution stirred for 30 minutes. TLC (6/1, methylene chloride/methanol) indicated some starting material present. An additional 1.0 ml of hydrogen peroxide was added and the solution stirred for 15 minutes. TLC indicated no starting material. The solvent was evaporated under reduced pressure to give a yellow foam. The foam was chromatographed on silica gel, using 3/1, methylene chloride/methanol, as eluting solvent. The appropriate fractions were combined and concentrated in vacuo to give 75 mg of the above-identified compound as a yellow foam.

HPLC (Waters C18, 100% 0.1N acetic acid, 1.0 ml/minutes, 260 nm) indicated 2 equimolar products. This is consistent with oxidation of the product to a diasteromeric mixture of sulfoxides.

¹H NMR (DMSO-d₆) Δ ppm, 2.6 (s, 3H, CH₃S(O)—), 3.0-3.2 (m, 2H, 5′-CH₂), 4.0-4.4 (m, 3H, 2′-CH, 3′-CH, 4′-CH) 5.4-5.6 (m, 3H, 2′-OH, 3′-OH, 1′-CH), 5.9 (br.s, 2H, 5-NH₂), 6.6-6.9 (br., 2H, 4-CONH₆), 7.3 (s, 1H, 2-CH).

### Example AS

### Preparation of 5-Amino-1-β-D-(5-deoxy-5-methylaminoribofuranosyl)imidazole-4-carboxamide (Compound No. 63 (1-517)

5′-Deoxy-5′-iodo-2′,3′-O-isopropylidene-AICA riboside (1.00 g) (ref: P. C. Srivastava, A. R. Newman, T. R. Mathews, and T. R. Mathews, and R. K. Robins, J. Med. Chem., 18, 1237 (1975)), methylamine 40% weight in water (3 ml), and methanol (30 ml) were combined and heated at reflux for 18 hours. The reaction gave a mixture of products. The solution was cooled and the solvents evaporated under reduced pressure. The resulting residue was chromatographed on silica gel, using 6/1 methylene chloride/methanol (400 ml) and 3/1 methylene chloride/methanol (300 ml) as the eluting solvent. The fractions containing the slow-eluting component which was desired product were combined and evaporated under reduced pressure to give 0.13 g of 5′-deoxy-5′-methylamino-2′,3′-isopropylidene-AICA riboside.

5′-deoxy-5′-methylamine-2′,3′-isopropylidene AICA riboside (0.13 g) was heated at 60° C. in 75% formic acid (20 ml) for 1.5 hour. The solution was cooled and the solvent evaporated under reduced pressure to yield a white foam. The foam was dissolved in water (5 ml) and applied to a short column of Dowex 50 strongly acidic ion-exchange resin. The column was washed with water then eluted with 1M NH₄OH in 20% methanol/water. The solvent was evaporated under reduced pressure and the resulting residue co-evaporated with methanol (3×20 ml) to yield 75 mg of the above-identified product as an off-white foam.

¹H NMR (D₆-DMSO-d) Δ ppm, 2.3 (s, 3H, CH₃N), 2.5-2.7 (m, 2H, 5′-CH₂), 3.3-3.4 (br., 1H, MENH), 3.9-4.3 (m, 3H, 2′-CH, 3′-CH, 4′-CH), 5.1-5.4 (m, 2H, 2′-OH, 3′-OH), 5.4 (d, 1H, 1′-CH), 6.2 (br.s, 2H, 5-NH₂), 6.6-6.8 (br., 2H, 4-CONH), 7.2 (s, 1H, 2-CH).

### Example AT

### Preparation of 5-Amino-1-β-D-ribofuranosylimidazole-4-N-(2-chlorophenyl)carboxamide (Compound No. 64 (1-519))

This compound was prepared according to the procedures described in Examples AK for compound 55 (1-484) for the 4-p-bromophenyl derivative, substituting 2-chloroaniline for 4-bromaniline. The final product was recrystallized from methylene chloride (20 ml)/methanol (1 ml) to yield 0.25 g of the above-identified product. Mp=131°-135° C.

¹H NMR (DMSO-d₆) Δ ppm, 3.5-3.6 (m, 2H, 5′-CH₂), 3.9-4.3 (m, 3H, 2′-CH, 3′-CH, 4′-CH), 5.2-5.4 (m, 3H, 2′-OH, 3′-OH, 5′-OH), 5.5 (d, 1H, 1′-CH), 6.2 (br.s, 2H, 5-NH₂), 7.0-8.4 (m, 5H, C₆H₄Br, 2′-CH), 9.1 (s, 1H, 4-CONH).

### Example AU

### Preparation of 5-Amino-1-β-D-(5-benzylamino-5-deoxyribofuranosyl)imidazole-4-carboxamide (Compound No. 66 (1-5311)

5′-deoxy-5′-iodo-2′,3′-isopropylidene AICA riboside (1.00 g) (ref: P. C. Srivastava, A. R. Newman, T. R. Mathews, and R. K. Robins, J. Med. Chem. 18: 1237 (1975)), benzylamine (2.0 ml), and methanol (40 ml) were combined and heated at reflux for 24 hours. Then, the procedures described in Example AS for Compound 63 (1-517) were followed to give the above-identified compound.

¹H NMR (DMSO-d₆) Δ ppm, 2.7 (d, 2H, —CH₂—C₆H₅), 3.3-3.4 (br., 1H, —NH—CH₂C₆H₅), 3.9-4.3 (m, 3H, 2′-CH, 3′-CH, 4′-CH), 5.1-5.4 (m, 2H, 2′-OH, 3′-OH), 5.4 (d, 1H, 1-CH), 6.1 (br.s, 2H, 5-NH₂), 6.6-6.8 (br., 2H, 4-CONH₂), 7.2-7.4 (m, 6H, —C₆H₅, 2-CH).

### Example AV

### Preparation of 5-Amino-2-thio-1-β-D-(5-deoxyribofuranosyl)imidazole-4-carboxamide (Compound No. 67 (1-535))

### A. Preparation of 5′-Deoxy-2′,3′-isopropylidene-2-bromo-AICA Riboside

To a solution of 5′-deoxy-2′,3′-isopropylidene-AICA riboside (2.90 g) (ref: P. C. Srivastava, A. R. Newman, T. R. Mathews, and R. K. Robins, J. Med. Chem., 18: 1237 (1975)) in chloroform (100 ml), was added N-bromosuccinimide in small portions over 20 minutes. The solution was stirred at ambient temperature for 30 minutes. The solution was washed with water, twice with brine, and then dried over magnesium sulfate. The solvent was evaporated in vacuo to yield a dark foam. The foam was passed through a column of silca gel, eluting with 9:1 methylene chloride:methanol. The fractions containing product were combined and concentrated under reduced pressure to yield 2.02 g of reddish-brown foam.

### B. Preparation of 5′-Deoxy-2-,3′-O-isopropylidene-2-thio AICA Riboside

Potassium sulfate (3.7 g) was heated at reflux in ethanol (20 ml) for 15 minutes. The mixture was filtered. To the filtrate was added 5′-deoxy-2′,3′-isopropylidene-2-bromo AICA riboside (from step A). The mixture was heated at 100° C. in a steel bomb for 5.5 hours. The mixture was cooled and filtered. The pH of the filtrate was adjusted to about 5-6 with acetic acid, and the solvent evaporated under reduced pressure. The resulting residue was passed through a column of silica gel, eluting with 7/1, methylene chloride/methanol. The fractions containing the product were combined and concentrated under reduced pressure to give a dark brown foam. The foam was stirred in methylene chloride (50 ml), then filtered to yield a pale purple powder. The powder was stirred in cold methanol, then filtered and vacuum dried to yield 0.52 g of a pale yellow solid. Mp=211-214 (decomposition).

### C. Preparation of 5-Amino-2-thio-1-(deoxy-β-D-ribofuranosyl)imidazole-4-carboxamide (Compound 67 (1-535))

5′-deoxy-2′,3′-isopropylidene-2-thiol AICA riboside (0.45 g) (from step B) was stirred in 50% formic acid (30 ml) at 50° C. for 1 hour. The solvent was evaporated under reduced pressure. The resulting residue was co-evaporated with methanol (2×20 ml). The resulting solid was warmed in methanol (25 ml), then stirred at room temperature overnight. The mixture was filtered and the filtrate concentrated under reduced pressure to yield a greenish foam. The foam was chromatographed on silica gel, using 5/1, methylene chloride/methanol, as the eluting solvent. The appropriate fractions were combined and concentrated under reduced pressure to give a yellow foam. The foam was crystallized from cold methanol to yield 69 mg. of the above-identified compound mp=201°-203° C., (decomposition).

¹H NMR (DMSO-d₆) Δ ppm 1.3 (d, 3H, 5′-CH₃), 3.6-4.5 (m, 3H, 2′-CH, 3′-CH, 4′-CH), 5.0-5.2 (m, 2H, 2′-OH, 3′-OH), 5.6 (br.s, 2H, 5-NH₂), 6.0 (d, 1H, 1′-CH), 7.0 (br., 2H, 4-CONH), 12.0 (br.s, 1H, —SH).

### Example AW

### Preparation of N,N′-bis-(5-amino-1-β-D-ribofuranosyl imidazole-4-carbonyl)-1,6-diaminohexane (Compound No. 68 (1-538))

N-succinimidyl-5-amino-1-(2,3,5-tri-O-acetyl-β-D-ribofuranosyl-imidazole-4-carboxylate (2.50 g) (ref: Srivastava, P. C., et al., J. Med. Chem. 17: 1207 (1974)), 1,6-hexane diamine (0.300 g), triethylamine (0.5 ml), and methylene chloride (35 ml) were combined and stirred at room temperature for 18 hours. The title compound was prepared according to the procedures described in Example J. The final product was crystallized from methanol to yield 0.32 g of the above-identified compound. Mp-181°-185° C.

¹H NMR data reported as for half the symmetrical dimer. ¹H NMR (DMSO-d₆) Δ ppm, 1.2-1.5 (m, 4H, β and Δ methylenes of N-hexyldicarboxamide), 3.0-3.2 (m, 2H, a methylene of N-hexyl dicarboxamide), 3.5-3.6 (m, 2H, 5′-CH₂), 3.8-4.3 (m, 3H, 2′-H, 3′-CH, 4′-CH), 5.1-5.4 (m, 3H, 2′-OH, 3′-OH, 5′-OH), 5.5 (d, 1H, 1′-CH), 5.9 (br.s, 2H, 5-NH₂), 7.3 (s, 1H, 2-Ch), 7.4 (t, 1H, 4-CONH).

### Example AX

### Preparation of N N′-Bis-(5-Amino-1-β-D-ribofuranosylimidazole-4-carbonyl)-1,4-diaminocyclohexane (Compound No. 69 (1-549))

This compound was prepared according to the procedures described in Example AW for compound 68 (1-538), substituting 1,4-diaminocyclohexane for 1,6-hexanediamine.

¹H NMR data are reported as for half the symmetrical dimer. ¹H NMR (DMSO-d₆) Δ ppm 1.3-1.8 (m, 4H, cyclohexane methylene protons), 3.5-3.7 (m, 3H, 5′-CH₂, cyclohexane methine), 3.8-4.3 (m, 3H, 2′-CH, 3′-CH, 4′-CH), 5.1-5.4 (m, 3H, 2′-OH, 3′-OH, 5′-OH), 5.5 (d, 1H, 1′-CH), 5.9 (br.s, 2H, 5-NH₂), 7.1 (d, 1H, 4-CONH) 7.3 (s, 1H, 2-CH).

### Example AY

### Preparation of 5-Amino-2-thio-1-(5-amino-5-deoxy-β-D-ribofuranosyl)imidazole-4-carboxamide (Compound No. 70 (1-551))

### A. Preparation of 5-Deoxy-5′-iodo-2-bromo-2′,3′-isopropylidene AICA Riboside

2-Bromo-2′3′-isopropylidene AICA riboside (4.50 g) (ref: T. Miyoshi, S. Suzaki, A. Yamazaki, Chem. Pharm. Bull. 29, 9: 2089, (1976) methyltriphenoxyphosphonium iodide (16.2 g), and methylene chloride (125 ml) were combined and stirred at room temperature for 16 hours. The mixture was washed with water, 0.5M NAOH (100 ml), 5% NaS₂O₃ (150 ml), and brine, then dried over magnesium sulfate. The solvent was evaporated under reduced pressure to give an orange oil. The oil was triturated in cold diethylether. The resulting mixture was filtered to give 3.53 g of a grey powder. The mother liquor was concentrated under reduced pressure to give an orange oil. The oil was applied to a short column of silica gel. The column was washed with methylene chloride, then the product eluted with 9/1, methylene chloride/methanol (250 ml). The appropriate fractions were combined and concentrated under reduced pressure to give an orange tar. The tar was triturated with cold diethyl ether. The mixture was filtered to yield an additional 0.94 g of a gray powder. The combined powder (4.47 g) was chromatographed on silica gel, using 2/1, ethylacetate/hexane, as eluting solvent. The appropriate fractions were combined and concentrated under reduced pressure to yield a yellow foam (4.02 g).

### B. Preparation of 5′-Azido-5′ deoxy-2-bromo-2′,3′-isopropylidene AICA Riboside

5′-deoxy-5′-iodo-2-bromo-2′,3′-isopropylidene AICA riboside (4.02 g) lithium azide (1.82 g), and DMF (65 ml) were combined and stirred at ambient temperature for 2 hours. The solvent was evaporated under reduced pressure to give a yellow oil. The oil was dissolved in ethyl acetate (200 ml), washed with water and brine, then dried over magnesium sulfate. The solvent was evaporated under reduced pressure to give a yellow foam (3.01 g).

### C. Preparation of 5′-Amino-5′-deoxy-2-bromo-2′,3′-isopropylidene AICA Riboside

5′-azido-5′-deoxy-2-bromo-2′,3′-isopropylidene AICA riboside (2.00 g), triphenylphosphine (1.83 g), and THF (100 g) were combined and stirred at room temperature for 16 hours. Concentrated NH₄OH (15 ml) was added and the solution heated at reflux for 6 hours. The solution was cooled and the solvent evaporated under reduced pressure. The resulting residue was coevaporated with methanol (2×30 ml). The resulting residue was stirred in cold methanol (25 ml) for 30 minutes. The mixture was filtered to give an off-white powder. The solid was recrystallized from methanol to give a white powder (0.73 g).

### D. Preparation of 5-Amino-2-thio-1-(5-amino-5-deoxy-β-D-ribofuranosyl)imidazole-4-carboxamide (Compound No. 70 (1-551))

Potassium sulfide (1.0 g) was heated at reflux in ethanol (10 ml) for 15 minutes. The mixture was filtered and to the filtrate was added 5′-amino-5′-deoxy-2-bromo-2′,3′-isopropylidene AICA riboside (0.50 g). The mixture was heated in a steel bomb at 110° C. for 5 hours. The mixture was cooled and filtered. The filtrate was again filtered, then concentrated under reduced pressure to give a yellow tar. The tar was chromatographed on silica gel, using 3/1, methylene chloride/methanol, as eluting solvent. The appropriate fractions were combined and concentrated under reduced pressure to give a yellow glass (0.12 g). The glass was dissolved in 80% of trifluoroacetic acid (8 ml) and stirred at room temperature for 1 hour. The solvent was evaporated under reduced pressure to give a yellow solid. The solid was stirred in diethylether/ethanol (10 ml of 95/5), then filtered and dried to yield a yellow solid (55 mg).

¹H NMR (DMSO-d₆+D₂O) Δ ppm, 2.6-2.9 (m, 2H, 5′-CH₂—), 3.8-4.5 (m, 3H, 2′-CH, 3′-CH, 4′-CH), 6.2 (d, 1H, 1′-CH).

### Example AZ

### Preparation of 5-Amino-1-(5-azido-5-deoxy-β-D-ribofuranosyl)imidazole-4-N->(4-nitrophenyl)methyl!carboxamide (Compound No. 71 (1-562))

This compound was prepared according to the procedures described in example AH for compound 52 (1-467), substituting compound 23 (1-343) (p-nitrobenzyl derivative), for compound 29 (1-349) (p-chlorobenzyl derivative).

¹H NMR (DMSO-d₆) Δ ppm, 3.5-3.7 (m, 2H, 5′-CH₂), 3.9-4.4 (m, 3H, 2′-CH, 3′-CH, 4′-CH), 4.4-4.5 (d, 2H, —CH₂-PhNO₂), 5.4-5.5 (m, 2H, 2′-OH, 3′-OH), 5.5 (d, 1H, 1′-CH), 5.9 (br.s, 2H, 5-NH₂), 7.4 (s, 1H, 2-CH), 6.5-8.2 (m, 4H, —C₆H₄NO₂), 8.3 (4, 1H, CONH—).

### Example BA

### Preparation of 5-Amino-1-(5-amino-5-deoxy-β-D-ribofuranosyl)imidazole-4-N->4-nitrophenyl)methyl!carboxamide (Compound No. 72 (1-563))

This compound was prepared according to the procedures described in Example AH for compared 53 (1-468), substituting the p-nitrobenzyl amide derivative (compound 23 (1-343)) for the p-chlorobenzyl amide derivative (compound 29 (1-349)).

¹H NMR (DMSO+D₂O) Δ ppm 2.6-2.8 (m, 2H, 5′-CH₂—), 3.8-4.3 (m, 3H, 2′-CH, 3′-CH, 4′-CH), 4.4-4.5 (m, 2H, —CH₂—C₆H₄NO₂), 5.4 (d, 1H, 1′-CH), 7.3 (s, 1H, 2-CH), 7.5-8.3 (m, 5H, CH₂ C₆H₄NO₂, 4-CONH).

### Example BB

### Preparation of 5-Amino-1-β-D-ribofuranosyl-imidazole-4-N->(4-(trifluoromethylphenyl)methyl!carboxamide (Compound No. 74 (1-572))

This compound was prepared according to the procedures described in Example J for the p-nitrobenzyl derivative substituting 4-(trifluoromethyl)benzylamine for 4-nitrobenzyl amine hydrochloride. The final product was recrystallized from methylene chloride/methanol. Mp=137-140.

¹H NMR (DMSO-d₆) Δ ppm 3.5-3.7 (m, 2H, 5′-CH₂), 3.9-4.4 (m, 3H, 2′-CH, 3′-CH, 4′-CH), 4.4-4.5 (d, 2H, —CH₂-PhCF₃), 5.2-5.5 (m, 3H, 2′-OH, 3′-OH, 5′-OH), 5.5 (d, 1H, 1′-CH), 5.9 (br.s, 2H, 5-NH₂), 7.3 (S, 1H, 2-CH), 7.4-7.7 (m, 4H, —C₆H₄CF₃), 8.2 (t, 1H, 4-CONH).

### Example BC

### Preparation of 5-Amino-1-β-D-ribofuranosylimidazole-4-N->(4-sulfamoylphenyl)methyl!carboxamide (Compound No. 75 (1-577))

This compound was prepared according to the procedures described in Example J for the p-nitrobenzyl derivative, substituting 4-(aminomethyl)benzene sulfonamide hydrochloride for 4-nitrobenzylamine hydrochloride.

¹H NMR (DMSO-d₆) Δ ppm, 3.5-3.7 (m, 2H, 5′-CH₂—), 3.9-4.4 (m, 3H, 2′-CH, 3′-CH, 4′-CH), 4.4-4.5 (d, 2H, —CH₂—C₆H₄ SO₂), 5.2-5.4 (m, 3H, 2′-OH, 3′-OH, 5′-OH), 5.5 (d, 1H, 1′-CH), 6.0 (br.s, 2H, 5-NH₂), 7.3 (br.s, 2H, —SO₂ NH₂), 7.4 (s, 1H, 2-CH), 7.4-7.8 (m, 4H, —C₆H₄—), 8.2 (t, 1H, 4-CONH—).

### Example BD

### Preparation of 5-Amino-1-(5-(4-chlorobenzyl-amino)-5-deoxyβ-D-ribofuranosyl)imidazole-4-carboxamide (Compound No. 76 (1-578))

5′-amino-5′-deoxy-AICA-riboside (0.50 g) (compound No. 21 (1-227)) of Table VIII, 4-chlorobenzyl iodide (0.50 g), potassium carbonate (0.26 g), and DMF (15 ml) were combined and stirred at room temperature for 16 hours. The solvent was evaporated under reduced pressure and the resulting residue stirred in warm ethanol (35 ml). The insolubles were removed by filtration and the filtrate concentrated under reduced pressure. The resulting residue was chromatrographed on silica gel, using 3:1, methylene chloride:methanol, as eluting solvent. The fractions containing the slower moving of the two products were combined and concentrated under reduced pressure to yield a tan foam (0.21 g)

sup.1 H NMR (DMSO-d₆+D₂) Δ ppm 2.9-3.0 (m, 2H, 5′-CH₂—), 3.9 (s, 2H, —CH₂—C₆H₄), 3.9-4.3 (m, 3H, 2′-CH, 3′-CH, 4′-CH), 5.5 (d, 1H, 1′-CH), 7.3 (s, 1H, 2-CH), 7.4 (m, 4H, —C₆H₄Cl).

### Example BE

### Preparation of 5-Amino-1-(5-deoxy-β-D-ribofuranosyl)imidazole; (Compound No. 77 (1-588))

5′-deoxy AICA riboside (1.00 g) (ref: P. C. Srivastava, A. R. Newman, T. R. Mathews, and R. F. Robins, J. Med. Chem. 18: 1237 (1975) was heated at reflux in N potassium hydroxide (4.0 ml) for 5 hours. The solvent was evaporated under reduced pressure and the resulting residue co-evaporated with ethanol (4×10 ml). The resulting residue was diluted with ethanol (15 ml) and a fine precipitate was filtered. Upon setting for several days, the filtrate gave an additional precipitate. The microscopic solid was collected, and the combined solid material was dissolved in water (20 ml) and neutralized with Dowex 50W strongly acidic ion exchange resin. The solvent was evaporated under reduced pressure to give a dark tar. The tar was dissolved in 80% acetic acid (20 ml) and gently heated (60° C.). The solvent was evaporated under reduced pressure to give a dark tar. The tar was co-evaporated with methanol (2×15 ml). The resulting residue was chromatographed on silica gel, using 3/1, methylene chloride/methanol, as eluting solvent. The appropriate fractions were combined and concentrated under reduced pressure to yield a dark tar. The tar was co-evaporated with toluene (3×20 ml), then vacuum dried to yield a dark brown, hygroscopic foam (110 mg).

¹H NMR (D₂) Δ ppm, 1.3 (d, 3H, 5′-CH₃), 4.0-4.5 (m, 3H, 2′-CH, 3′-CH, 4′-CH), 5.6 (d, 1H, 1′-CH), 6.4 (s, 1H, 4-CH), 7.7 (s, 1H, 2-CH).

### Example BF

### Preparation of 5-Amino-1-(5-deoxy-5-diethylaminoribo-β,D-furanosyl)imidazole-4-carboxamide (Compound No. 65 (1-522)

5-deoxy-5′-iodo-2′,3′-isopropylidene AICA riboside (1.00 g) (ref.: P. C. Srivastava, A. R. Newman, T. R. Mathews, and R. K. Robins, J. Med. Chem. 18: 1237, (1975)), diethylamine (2.5 ml of 40 wt % in water), and methanol (30 ml) were combined and heated at reflux for 18 hours. The procedures described in Example AS for compound 63 (1-519) were followed to give the above-identified compound.

¹H NMR (DMSO-d₆) Δ ppm 0.9 (t, 6H, methyl groups on 5′-diethylamine), 2.4-2.7 (m, 6H, 5′-CH₂, methylene groups on 5′-diethylamine), 3.3-4.2 (m, 3H, 2′-CH, 3′-CH, 4′-CH), 5.2 (br., 2H, 2′-OH, 3′-OH), 5.4 (d, 1H, 1′-CH), 5.9 (br.s, 2H, 5-NH₂), 5.7-5.9 (br., 2H, 4-CONH₂), 7.3 (s, 1H, 2-CH).

### Example BG

### Preparation of 5-Amino-1-β-D-ribofuranosylimidazole-4-N->3-4-nitrophenyl)propyl!carboxamide (Compound No. 73 (1-566))

This compound was prepared according to the procedures described in Example J for the p-nitrophenyl derivative, substituting 3-(4-nitrophenyl)propylamine (ref: G. W. Hardy, et al., J. Med. Chem. 32: 1108, (1989)) for p-nitrobenzylamine hydrochloride.

¹H NMR (DMSO-d₆) Δ ppm 1.7-3.2 (m, 6H, —CH₂ CH₂—), 3.5-3.6 (m, 2H, 5′-CH₂), 3.9-4.3 (m, 3H, 2′-CH, 3′-CH, 4′-CH), 5.2-5.4 (m, 3H, 2′-OH, 3′-OH, 5′-OH), 5.5 (d, 2H, 1′-CH), 5.9 (br.s, 2H, 5-NH₂), 7.3 (s, 1H, 2-CH), 7.5-8.2 (m, 5H, —CH₆H₄NO₂, 4-CONH—).

### Example BH

### Preparation of 5-Amino-1-(5-amino-5-deoxy-2,3-di-O-acetyl-β-D-ribofuranosyl)imidazole-4-N->(4-chlorophenyl)methyl!carboxamide, (Compound No. 78 (1-599))

### A. Preparation of 5-amino-1-(5-azido-5-deoxy-2,3-di-O-acetyl-β-D-ribofuranosyl)imidazole-4-N->4-chlorophenyl)methyl!carboxamide

Compound 52 (example AH), 2.4 g (5.8 mmol), was dissolved in a mixture of 20 ml of diemthylformamide and 20 ml of pyridine. The solution was cooled to 30° C. under argon, and acetic anhydride, 1.5 g, (14 mmol), was added. The mixture was allowed to warm to room temperature over 18 hours and then concentrated to a syrup. The syrup was dissolved in 25 ml of methylene chloride and washed with 3×15 ml of water, dried over magnesium sulfate and evaporated to yield 3.0 grams of a white foam. This was further purified by chromatography on 200 ml of silica gel using a mixture of methylene chloride and methanol (95:5), yielding 2.5 grams of the desired product as a white foam.

### B. Preparation of 5-amino-(5-amino-5-deoxy-2,3-di-O acetyl-β-D-ribofuranosyl)imidazole-4-N->(4-chlorophenyl)methyl!carboxamide (Compound No. 78 (1-599))

The product of step A, 400 mg, was dissolved in 10 ml of ethanol and 50 mg of 10% Pd on carbon was added. The mixture was stirred under a hydrogen atmosphere for 30 minutes, filtered and the filtrate evaporated to yield 300 mg of the desired product as a white foam.

¹H NMR (DMSO-d₆) Δ 2.0 (s, 3H, CH₃ CO—), 2.1 (s, 3H, CH₃ CO—), 2.9 (m, 2H, 5′-CH₂), 4.1 (m, 1H, 4′-CH), 3.4 (br. s, 2H, 5′—NH₂) 4.4 (d, 2H, —CH₂—C₆H₄—Cl), 5.3 (m, 1H, 3′-CH) 5.6 (m, 1H, 3′-CH), 5.8 (d, 1H, 1′-CH), 6.4 (br. s, 2H, 5-NH₂), 7.3 (m, 4H, —C₆H₄—Cl), 7.4 (s, 1H, 2-CH), 8.1 (t, 1H, 4-CONH—).

### Example BI

Prodrugs of the invention can also be prepared and administered under appropriate conditions. In a preferred embodiment, the prodrugs of the invention enhance oral bioavailability, and include in particular the carboxylic acid esters of 2′ and 3′ hydroxyls.

Prodrug esters of the invention can be made by standard acetylation procedures, which may involve protection and deprotection steps. For example, the 5′ group of Series III compounds may require protection (e.g., the 5′-benzylamino of Compound 66 can be protected with a benzyloxycarbonyl group.)

### Preparation of 5-Amino-1-(5-N-benzylamino-2,3,-di-O-pivaloyl-5-deoxy-β-D-ribofuranosyl)imidazole-4-carboxamide (Prodrug of Compound 66)

1-(5-N-benzylamino-5-deoxy-β-D-ribofuranosyl)imidazole-4-carboxamide tartrate salt (8.8 g, 16.77 mmol) in water (60 mL), potassium carbonate (8.5 g), and tetrahydrofuran (120 mL) was taken in a three-necked round bottom flask fitted with a mechanical stirrer, an addition funnel, and a nitrogen inlet. The flask was cooled in an ice water bath. A solution of benzyl chloroformate (3.4 mL, 20 mmol) in THF (15 mL) was added over a period of 15 minutes. The cooling bath was removed and stirring was continued for two hours, at which time t.l.c. (SiO₂, 6:1 CH₂ Cl₂-Methanol) indicated complete consumption of the starting material. The reaction mixture was transferred to a separatory funnel and the organic layer was separated. The aqueous layer was washed with ethyl acetate (3×30 ml). The organic layers were combined, dried over anhydrous MgSO₄ and evaporated to obtain a syrupy residue. The product was further purified by column chromatography using 9:1 CH₂ Cl₂-Methanol as the eluting system. Fractions containing the product were pooled and evaporated to obtain

### A. 5-amino-1-(5-N-benzylamino-N-benyoxycarbonyl-5-deoxy-β-D-ribofuranosyl)imidazole-4-carboxamide as a glassy solid. Yield: 5.5 g. R_{f}=0.5 SiO₂, 6:1 CH₂ Cl₂-Methanol

A solution of compound A (2.0 g, 4.15 mmol) and 4-N,N-dimethylaminopyridine (100 mg) in dry pyridine (20 mL) was cooled in an ice water bath and treated with pivalic anhydride (3.3 mL). The ice bath was removed and the reaction mixture was stirred at room temperature for 16 hours. The t.l.c. (SiO₂, 9:1 CH₂ Cl₂-Methanol) indicated complete consumption of the starting material. Methanol (1.5 mL) was added and stirred for an additional half-hour, and the volatiles were evaporated under reduced pressure. The residue was dissolved in ethyl acetate (50 mL) and extracted with water (1×50 mL) and sodium bicarbonate solution (1×20 mL). The organic layer was dried over anhydrous MgSO₄ and evaporated to obtain a syrupy residue. The product was further purified by column chromatography using 19:1 CH₂ Cl₂-Methanol as the eluting system. Fractions containing the product were pooled and evaporated to obtain

### B. 5-amino-1-(5-N-benzylamino-N-benyoxycarbonyl-2,3-di-O-pivaloyl-5-deoxy-β-D-ribofuranosyl)imidazole-4-carboxamide as a glassy solid. Yield: 5.5 g. R_{f}=0.6 SiO₂, 9:1 CH₂ Cl₂-Methanol. HNMR, DMSC-d₆ Δ ppm

To a solution of compound B (1.1 g) in ethyl acetate (30.0 mL) and acetic acid (6.0 mL) the catalyst Pd(OH)₂ on carbon (100 mg) was added and purged with nitrogen. Hydrogenation was carried out using a balloon of hydrogen. Completion of the reaction was evidenced by the absence of starting material on t.l.c. (SiO₂, 9:1 CH₂ Cl₂-Methanol). The catalyst was removed by filtration through a celite pad and washed with ethyl acetate. The filtrate was evaporated under reduced pressure and the residue was redissolved in ethyl acetate (50 mL) and extracted with saturated sodium bicarbonate solution (1×20 mL). The organic layer was dried over anhydrous MgSO₄ and evaporated to obtain a residue which was further purified over a silica gel column using 19:1 CH₂ Cl₂-Methanol as the eluting system. Fractions containing the product were pooled and evaporated to obtain

### C. 5-amino-1-(5-N-benzylamino-N-benzylamino-2,3-di-O-pivaloyl-5-deoxy-β-D-ribofuranosyl)imidazole-4-carboxamide as a glassy solid. Yield: 800 mg. R_{f}=0.55 SiO₂, 9:1 CH₂ Cl₂-Methanol

To obtain the corresponding hydrochloride salt of the title compound, the above free base (200 mg) was dissolved in methanol and diluted with 1N aqueous HCL solution. The resulting solution was evaporated under reduced pressure (bath temperature, 30 C.). The residue was dissolved in double distilled water (15 mL) and filtered through a 45μ membrane filter. The filtrate was frozen in a lyophilizing jar and lyophilized repeatedly until a constant weight was obtained. The final product 5-amino-1-(5-N-benzylamino-N-benzylamino-2,3-di-O-pivaloyl-5-deoxy-β-D-ribofuranosyl)imidazole-4-carboxamide hydrochloride was obtained as a white solid was dried under high vacuum and stored in the freezer. Yield: 180 mg, m.p. 172°-175° C.

The following prodrugs can be made in a similar manner:
5-amino-1-(5-N-benzylamino-2,3-di-O-acetyl-5-deoxy-β-D-ribofuranosyl)imidazole-4-carboxamide
5-amino-1-(5-N-benzylamino-2,3-di-O-propionyl-5-deoxy-β-D-ribofuranosyl)imidazole-4-carboxamide
5-amino-1-(5-N-benzylamino-2,3-di-O-butyryl-5-deoxy-β-D-ribofuranosyl)imidazole-4-carboxamide
5-amino-1-(5-N-benzylamino-2,3-di-O-isobutyryl-5-deoxy-β-D-ribofuranosyl)imidazole-4-carboxamide
5-amino-1-(5-N-benzylamino-2,3-di-O-pentanoyl-5-deoxy-β-D-ribofuranosyl)imidazole-4-carboxamide
5-amino-1-(5-N-benzylamino-2,3-di-O-benzoyl-5-deoxy-β-D-ribofuranosyl)imidazole-4-carboxamide
5-amino-1-(5-N-benzylamino-2,3-di-O-(4-methylbenzoyl)-5-deoxy-β-D-ribofuranosyl)imidazole-4-carboxamide
5-amino-1-(5-N-benzylamino-2,3-di-O-phenylacetyl-5-deoxy-β-D-ribofuranosyl)imidazole-4-carboxamide
5-amino-1-(5-N-benzylamino-2,3-di-O-palmitoyl-5-deoxy-β-D-ribofuranosyl)imidazole-4-carboxamide
5-amino-1-(5-N-benzylamino-2,3-di-O-oleyl-5-deoxy-β-D-ribofuranosyl)imidazole-4-carboxamide
5-amino-1-(5-N-benzylamino-5-deoxy-β-D-ribofuranosyl)imidazole-4-carboxamide-2′,3′-cyclic carbonate.

### Example BJ

### Adhesive Patch Preparation

PSA Compositions Prepared by Mixing Different Hydrophilic Polymers with Complementary Short-chain Plasticizers

PVP as well as a a range of different hydrophilic polymers can be utilized to form drug delivery systems of the invention. PVP or other polymers can become tacky upon mixture with short-chain plasticizers bearing complementary reactive groups at the chain ends. Suitable hydrophilic polymers include poly(N-vinyl amides) such as PVP (Examples 1-47), poly(N-vinyl caprolactam) (PVCap) (Examples 48-52) and poly(N-vinyl acetamide) (PVAA, Example 53), poly(N-alkyl acrylamides), exemplified by poly(N-isopropyl acrylamide) (PNIPAM, Example 54), polymethacrylic and polyacrylic acid (PMA, PAA, Examples 55-60), and the copolymers thereof exemplified in the Table 4 (Luviscols VAP.®, commercially available from BASF; Examples 61-64). Luviscol VAP 37 is a copolymer of vinylpyrrolidone (VP, 30%) with 70% vinylacetate (VA). Luviscol VAP 73 contains 70% of VP and 30% of VA.

For example, PEG-400 blends with PVCap can be utilized to provide a drug dosing form. PVPCap takes up nearly four times less water than PVP under comparable conditions. According to the Zaikov-Iordanskii-Markin classification of polymer hydrophilicity, PVP is hygroscopic, whereas PVCap is a moderately hydrophilic polymer. Compared to a PVP blend with 36 wt. % of PEG-400, the PVCap plasticized with the same amount of PEG-400 will provide higher adhesion. Furthermore, an increase in polymer molecular weight will result in an increase in cohesive toughness and adhesion. The sorption and adhesive properties of PVCap-PEG hydrogels are thus ideal for pharmaceutical applications. It should also be noted that PVCap exhibits a Lower Critical Solution Temperature (LCST) about 35° C. (see Kirsh Y. E., Water soluble poly(N-vinylamides), Wiley, N.Y., 1998). Below this temperature the PVCap is easily soluble in water and behaves like moderately hydrophilic polymer, while above LCST the PVCap becomes insoluble in water hydrophobic polymer. This property is highly useful for the development of thermoresponsive or so-called “smart” adhesive hydrogels.

In addition to pressure-sensitive adhesion, the adhesive hydrogels for use with the compounds disclosed herein can display a number of useful viscoelastic properties, similar to the properties of cured rubbers. In fact, adhesive hydrogels are hydrophilic and water-soluble rubbers, featuring rubber-like elasticity as one of their major performance properties. Both the rubber-like elastic and viscous contributions to the rheology of hydrogelsrender such adhesive hydrogels as ideal for use in a patch drug delivery system.

For example, under a fixed compressive force, hydrogel squeezing occurs, and the instantaneous compressibility (Hooke's elasticity) of the PVP-PEG adhesive hydrogel is proportional to the applied compressive load. Hooke's compression is followed by a squeezing flow (creep), which reflects the viscous flow contribution to hydrogel rheology. As compression is developing, the stress within the adhesive polymer decreases gradually, achieving a critical value of yield stress at which the hydrogel ceases to flow and the squeezing flow is stopped. Occurrence of yield stress is a characteristic feature of crosslinked or highly ordered supramolecular structures, typical of polymer networks or liquid-crystalline polymers. Commodity polymers exhibit no yield stress. The yield stress in the PVP-PEG adhesive hydrogel is associated with a critical magnitude of shear stress in the flow curve, at which the apparent shear viscosity climbs to infinity and the deformation rate drops tending to zero. The yield stress is an integral measure of cohesive strength, which has been shown to determine the adhesive properties of an adhesive polymer (Feldstein et al. (2000), “Molecular insight into theological and diffusion determinants of pressure-sensitive adhesion,” Proceed. 23^{rd} Annual Meeting Adhesion Soc., pp. 54-56).

Upon removal of compressive load, the relaxation of polymer deformation occurs, termed the “retardation,” when deformation changes its sign and the polymer returns, more or less, to its initial shape. The Hooke's elastic retardation value is generally proportional to compressive force, and the slope of this linear relationship outlines the elastic relaxation shear modulus of adhesive hydrogel G=2×10⁵ Pa. This value found for the PVP-PEG adhesive hydrogel is well known to be also inherent in various PSA polymers and slightly crosslinked rubbers.

Upon the removal of compressive bonding force, the adhesive hydrogel is allowed to relax and creep recovery occurs, accompanied by a gradual growth in creep compliance. Kinetics of PVP-PEG hydrogel creep recovery, described by creep-recoil function S(t), follow the phenomenological Dickie-Ferry equation, based on the model developed to describe the relaxation of slightly crosslinked rubbers.

### Example BK

### Incorporation of Therapeutic Agents into Patch Systems

In some embodiments, 5-amino-1-β-D-(5-benzylamino-5-deoxy-1-β-D-ribofuranosyl)imidazole-4-carboxamide, 5-amino-1-(5-amino-5-deoxy-β-D-ribofuranosyl)imidazole-4-N-[(4-chlorophenyl)methyl], N-(4-chlorobenzyl)-5-amino-1-b-D-(5-amino-5-deoxy-1-b-D ribofuranosyl)imidazole-4-carboxamide or 5-[N-(4-nitrobenzyl)-5-amino-1-b-D-(1-b-D ribofuranosyl)imidazole-4-carboxamide]phosphate or a combination of two or more of the same, can be comprised in a patch for delivery to a subject.

Such therapeutic agents (drugs) can be first dissolved in a mixture of PEG-400 (plasticizer) and ethyl alcohol (solvent). Upon complete drug dissolution, the PVP or another hydrophilic polymer can be dissolved in the earlier prepared mixture, forming casting solution that is cast onto polyethylene terephthalate backing film of 0.02 mm in thickness and dried at temperatures of 20-60° C.

In vitro drug delivery rate determination from water-soluble PSA matrices can be performed using human cadaver skin or a skin-imitating Carbosil membrane to protect the matrices from dissolving in the receptor solution. The patch can be adhered to the center of a Carbosil membrane sheet of twice its area. The membrane margins can be then wrapped around the sample. The back side of the packet can be closely attached to a steel plate-holder to prevent direct contact of the matrix with the receptor solution. In a similar fashion, cadaver skin epidermis can be used instead of a Carbosil membrane. The holder with the wrapped sample can then be submerged into an aqueous sink and in vitro drug delivery rate determined using a USP rotating cylinder method paddle-over-disc. The rate of drug appearance rate in a receptor solution (0.15 M NaCl) at 35.0.+−.0.5° C. can be determined spectrophotometrically.

Drug permeability coefficients through skin epidermis (P.sub.s) and a Carbosil membrane (P.sub.m) can be measured as the fluxes of drug delivered from the hydrophilic PSA matrix normalized by the drug concentration in the donor vehicle, taking the matrix density (1.10.+−.0.12 g/cm³) into account.

### Example BL

### Heating Component(s)

Patch systems described above, can also comprise a penetration enhancer, such as a chemical compound or a heater component to enhance absorption/delivery of the drug compound.

For example, the drugs of the invention may be incorporated into a controlled release polymer matrix, such as homopolymer or copolymer such as those described in Examples BJ, BK, or a lactic and glycolic acid, preferably poly(DL-lactide), poly(DL-lactide-co-glycolide), and poly(DL-lactide-co-(-caprolactone)), to increase the duration of action. Where a heating component is utilized, the heating patch quickly increases the temperature of the injection site to a narrow range, and maintains it therefore a desirable duration of time (e.g., about 8 hours, 9, 10, 11, 12, 13, 14, 15, 16 or 18 hours). The heating causes increased release, and, thus, higher blood concentrations. Where the heating component or drug matrix component are separable, the “used-up” patch can be removed before a new heating patch is placed on the same. Using this intermittent heat application technique, blood concentrations can be modulated over time; for example, concentrations are low in the night and high in the day, correlating to application of the heating component.

All references cited herein are hereby incorporated by reference in their entireties.

It should be noted that where the terms “AICA riboside” or “acadesine” appear throughout, each may be interpreted to mean acadesine, a prodrug, analog, or salt thereof.

The above examples are in no way intended to limit the scope of the instant invention. Further, it can be appreciated to one of ordinary skill in the art that many changes and modifications can be made thereto without departing from the spirit or scope of the appended claims, and such changes and modifications are contemplated within the scope of the instant invention.

What is claimed is:

1. A transdermal drug delivery system that substantially maintains contact with a mucous or dermal layer, wherein said system comprises a drug dosing form comprising an AICA riboside compound or analogs thereof, wherein said compound comprises a general formula: 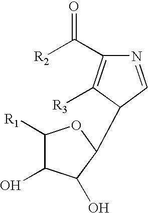
wherein R₁ is a phosphate group, CH₂, CO, CH₂NH, —NHCH₂, —CH₂NHCH₂, CH₂NHCH₂R₄, —CH₂—N(R₅)CH₂—, —CO—CO—, CH₂(R₅)N—, CH(R₅), S, CH₂(CO), N(R₅) wherein R₅ is hydrogen, optionally substituted C₁₋₁₂ alkyl, C₃₋₁₂ cycloalkyl, C₁₋₆ alkoxy, C₁₋₆ alkyl, aryl or heteroaryl, where R₁ is not a hydroxyl group;
R₄ is a monocyclic ring comprising 5-7 atoms;
R₃ is NH₂;
R₂ is CONH₂, CONHCH₂R₆; wherein R₆ is a monocyclic ring comprising 5-7 atoms.

2. The system of claim 1, wherein said delivery comprises using a patch.

3. The system of claim 2, wherein said compound is comprised in a reservoir or an adhesive layer comprised in said patch.

4. The system of claim 1, wherein said system maintains contact with a mucous layer.

5. The system of claim 1, wherein said system maintains contact with a dermal layer.

6. The system of claim 1, wherein said dosing form is delivered over a controlled period of time and at a controlled concentration.

7. The system of claim 1, further comprising a permeation enhancer component.

8. The system of claim 1, further comprising an anti-initant component.

9. The system of claim 7, wherein said enhancer component comprises a biological, chemical or physical means to enhance said delivery.

10. The system of claim 7, wherein said enhancer component provides heat for a controlled period of time and at a desired temperature.

11. The system of claim 1, wherein said dosing form is administered in amounts ranging from about 20-1500 mg/hr.

12. The system of claim 1, wherein said drug dosing form is administered in amounts ranging from about 0.1 mg/kg/day to about 500 mg/kg/day.

13. The system of claim 1, wherein said drug dosing form is administered in amounts ranging from about 0.01 mg/kg/min to about 2.0 mg/kg/min; from about 0.05 mg/kg/min to about 0.2 mg/kg/min; or from about 0.1 mg/kg/min to bout 0.125 mg/kg/min.

14. The system of claim 7, further comprising an anti-irritant component.

15. The system of claim 1, wherein said dosing form comprises about 1-50% by weight of said AICA riboside or analogs thereof.

16. The system of claim 1, wherein said drug dosing form comprises said AICA riboside or analogs thereof and a second compound.

17. The system off claim 16, wherein said second compound comprises a second AICA riboside or analogs thereof, an enhancer of AICA synthesis, an enhancer of AICA buildup or an enhancer of bacterial AICA production or an anti-clotting agent.

18. The system of claim 16, wherein said second compound comprises allopurinol.

19. A transdermal drug delivery system that substantially maintains contact with a mucous or dermal layer, wherein said system comprises comprising a drug dosing form comprising an AICA riboside compound or analogs thereof, wherein said compound has a general formula: 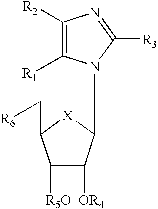
or a pharmaceutically acceptable salt thereof wherein X is —O— or —CH₂—; R₁ is hydrogen, amino, hydrocarbylamino, acylamino, or dihydrocarbylaminoalkyleneimino; R₂ is hydrogen, cyano, hydrocarbylimidate, carboxamideoxime, hydrocarbyloxyamidine, carboxamide, or carboxylic acid or an amide, ester, thioester or salt thereof; R₃ is hydrogen, hydrocarbyl, amino, hydrocarbylamino, halogen, hydroxy (including tautomeric 2-imidazolone), hydrocarbyloxy, sulfhydryl (including tautomeric 2-imidazolthione), or hydrocarbylthio; R₄ and R₅ are independently hydrogen, alkyl, acyl or hydrocarbyloxycarbonyl; R₆ is hydrogen, hydrocarbyl, halogen, hydroxy, hydrocarbyloxy, sulfhydryl, hydrocarbylthio, sulfamyloxy, amino, hydrocarbylamino, azido, acyloxy or hydrocarbyloxycarboxy or phosphate ester group or salts thereof; provided that when R₁ is amino, R₂ is unsubstituted carboxamide, R₃ is hydrogen; R₄ and R₅ are hydrogen, acyl or hydrocarboxycarbonyl; then R₆ is not hydroxy, acyloxy or hydrocarbyloxycarboxy.

20. The system of claim 3, wherein said compound comprises: 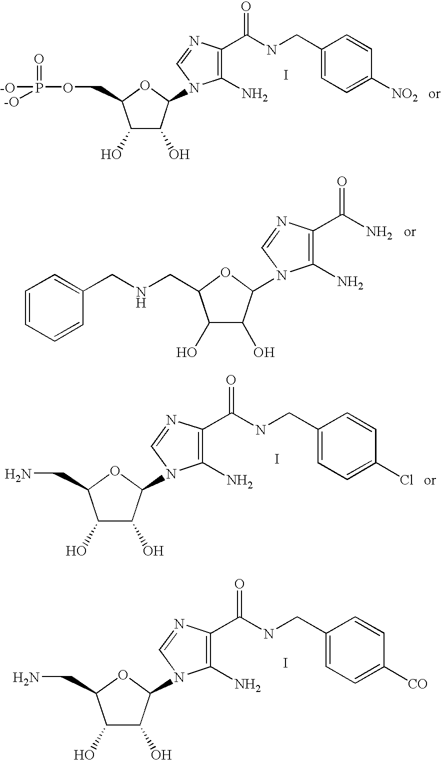
or a combination thereof.

21. A method of treating a subject comprising administering transdermally to said subject an AICA riboside compound or analogs thereof to said subject to reduce ischemiarelated disorders, wherein said compound comprises: 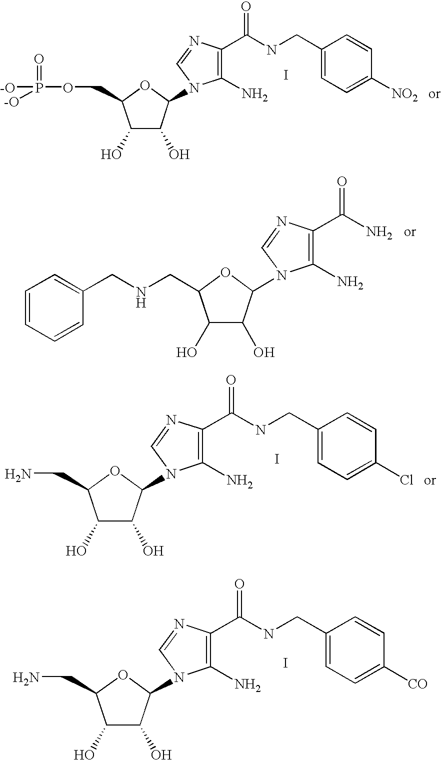
or a combination thereof.

22. The method of claim 21, wherein said treatment comprises administering an AICA riboside compound or analogs thereof comprises delivery by a route selected from intravenous, oral, transdermal, subcutaneous, perfusion, or a combination thereof.

23. The method of claim 21, wherein said administering results in increased local levels of adenosine.

24. The method of claim 21, wherein said administering results in increasing blood flow to an ischemic site.

25. The method of claim 21, wherein said administering is at a controlled rate of delivery over a period of time.

26. The method of claim 25, wherein said period of time is from 1 to 100 days.

27. The method of claim 25, wherein said administering comprises a transdermal patch.

28. The method of claim 27, wherein said transdermal patch comprises at least one reservoir or adhesive layer.

29. The method of claim 28, wherein said reservoir or adhesive layer comprises said AICA riboside compound or analogs thereof.

30. The method of claim 29, further comprising administering one or more additional compounds.

31. The method of claim 30, wherein said one or more additional compounds comprises a second AICA compound, a permeation enhancer, an anti-irritant, allopurinol, a blood clotting inhibitor, an enhancer of AICA synthesis, an enhancer of AICA buildup or an enhancer of bacterial AICA production or a combination thereof.

32. The method of claim 21, wherein said administering is before or after surgery.

33. The method of claim 32, wherein said administering is within 6 days, 5 days, 4 days, 3 days, 2 days or 1 day perioperatively.

34. The method of claim 32, wherein said administering is within 48 hours, 36 hours, 24 hours, 12 hours, 8 hours, 6 hours or 1 hour perioperatively.

35. The method of claim 32, wherein said surgery comprises cardiac, abdominal, neurological, gynecological, orthopedic, urological, vascular, and surgery related to otolaryngology.

36. The method of claim 21, wherein said administering transdermally comprises a component for enhancing said drug delivery.

37. The method of claim 36, wherein said component comprises a biological, chemical or physical means for said enhancing of said drug delivery.

38. The method of claim 36, wherein said component provides heat for a controlled period of time at a desired temperature.

39. The method of claim 36, wherein said enhancing results in modulation of blood flow.

40. A drug delivery patch that substantially maintains contact with a mucous or dermal layer, wherein said patch comprises a drug dosing form of acadesine.

41. The patch of claim 40, further comprising an enhancer component that enhances delivery of said acadesine.

42. The patch of claim 41, wherein said enhancer said enhancer component comprises a biological, chemical or physical means to enhance said delivery.

43. The patch of claim 41, wherein said enhancer component provides heat for a controlled period of time and at a desired temperature, whereby said heat enhances said acadesine delivery.

44. The patch of claim 41, wherein said patch comprises at least one adhesive layer.

45. The patch of claim 41, wherein said acadesine is contained in an adhesive layer component, wherein said adhesive layer component is in direct contact with said mucous or dermal layer.
